(19) United States
(12) Patent Application Publication
(10) Pub. No.: US 2025/0188102 A1
(43) Pub. Date: Jun. 12, 2025

(54) TRIAZINE COMPOUND, INTERMEDIATE THEREOF, PREPARATION METHOD THEREFOR AND USE THEREOF

(71) Applicant: JKT BIOPHARMA CO., LTD., Dongxihu District, Wuhan, Hubei (CN)

(72) Inventors: Xiaokun SHEN, Shanghai (CN); Jinwen HUANG, Shanghai (CN)

(21) Appl. No.: 18/845,444

(22) PCT Filed: Mar. 10, 2023
(86) PCT No.: PCT/CN2023/080920

§ 371 Date: Oct. 10, 2024

(30) Foreign Application Priority Data
Mar. 10, 2022 (CN) 202210245736.X
May 22, 2022 (CN) 202210557410.0

(51) Int. Cl. C07F 9/6558; C07D 403/14

(52) U.S. Cl. CPC C07F 9/65583; C07D 403/14

(57) ABSTRACT

A triazine compound, an intermediate thereof, and a preparation method therefor and the use thereof. Specifically, the present invention relates to a triazine derivative as represented by formula (I′) or a pharmaceutically acceptable salt thereof. On the basis of retaining the effectiveness against SARS-CoV-2, the compound significantly prolongs the half-life period, reduces the dosage requirement, reduces side effects, expands the range of therapeutic window, and has very good prospects when used for manufacturing a drug for treating diseases related to coronavirus infections.

This application claims priority for the Chinese patent application 202210245736X with a filing date of 2022 Mar. 10 and the Chinese patent application 2022105574100 with a filing date of 2022 May 20. This application quotes the full text of the above-mentioned Chinese patent application.

### TECHNICAL FIELD

The invention relates to a triazine compound, an intermediate thereof, and a preparation method thereof and an application thereof.

### BACKGROUND TECHNIQUE

Coronaviruses (CoV) are a class of pathogenic microorganisms that are seriously harmful to humans. To date, a total of seven coronaviruses that can infect humans have been found, namely SARS-CoV, MERS-CoV, SARS-CoV-2 (novel coronavirus, also known as 2019-nCoV), HCoV-229E, HCoV-OC43, HCoV-NL63 and HCoV-HKUI. The disease caused by the novel coronavirus (COVID-19) is caused by infection with the novel coronavirus (SARS-CoV-2). The general symptoms of SARS-CoV-2 infection are fever, fatigue, dry cough, and progressive dyspnea. some patients have mild onset symptoms, or even no obvious fever. Severe symptoms include: Acute respiratory distress syndrome, septic shock, refractory metabolic acidosis, and coagulation dysfunction. SARS-CoV-2 virus positive can be detected in nasopharyngeal swabs, sputum, respiratory secretions, blood, feces, etc., in patients infected with SARS-CoV-2. Chest imaging shows multiple small spots and interstitial changes in the early stage. It is obvious in the outer lung zone. Further, it develops into multiple grinding glass shadows and infiltrating shadows in both lungs. In severe cases, lung consolidation may occur, and pleural effusion is rare. So far, more than 400 million people have been infected worldwide, with more than 6 million cumulative deaths.

Although many vaccines have been approved and large-scale vaccination has begun, infections after vaccination are not uncommon due to the mutation of the virus and the protective effectiveness of the vaccine. At present, among the targeted drugs of COVID-19 virus, Gilead RNA polymerase (RdRp) inhibitor Radesivir and Merck CoV oral drug molnupiravir have been approved for marketing. Roche and REGN-COV2 (casirivimab/imdevimab), a regeneration-targeted S-protein cocktail, have been granted Emergency Use authorization (EUA) in the United States.

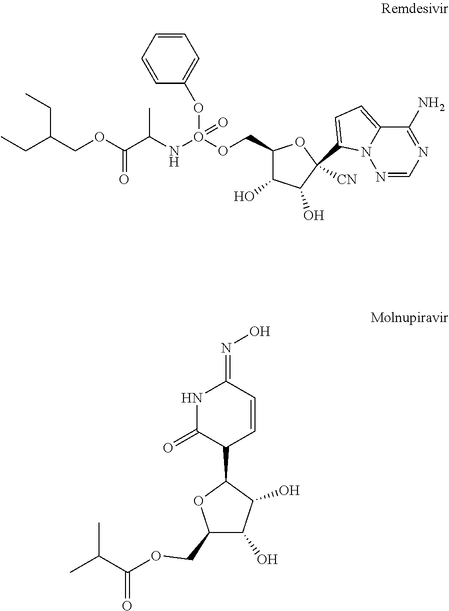

3CL protease (3C-likeprotease, 3CLpro) is the main protease that cuts and processes RNA in the virus's own coding, and it is the main protease produced by novel coronavirus (2019-nCoV, SARS-CoV-2). It plays a key role in the replication process of coronavirus, and its sequence is highly conserved. It is a popular target for anti-coronavirus drug research and development. At present, many 3CL protein inhibitors are in the clinical research and development stage. Among them, Pfizer's polypeptide 3CL protease inhibitor PF-07321332 has a single drug inhibitory activity on the virus at the molecular level: IC50=19 nM. PF-07321332 single drug pair in human airway epithelial cells, HeLa and A549 cells expressing ACE2 protein viral inhibitory activity: EC50 is 62, 99 and 56 nM respectively, showed good clinical effects, and its compound preparation Paxlovid (PF-07321332/Ritonavir) was approved by the FDA for emergency use. S-217622 is a non-peptide small molecule 3CL protein inhibitor developed by Shionogi Company of Japan. It has been found in vitro experiments and has inhibitory activity against SARS-Cov-2, SAR, MERS and human coronavirus HCoV-229E, etc. It is effective against the mutation of novel coronavirus and has stronger inhibitory activity against Omicron strain. S-217622 is now in Phase 2-3 clinical trials.

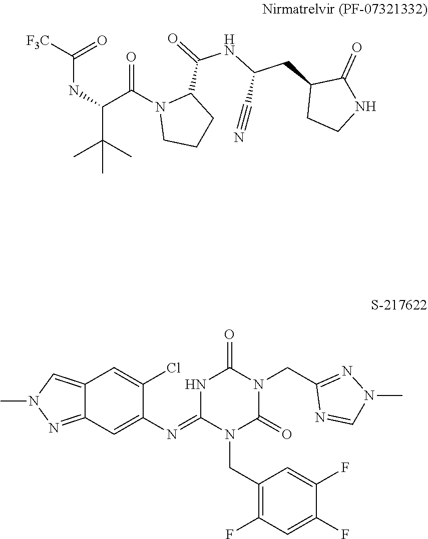

At present, there are no reports on the deuterated products and prodrugs of S-217622 compound and its analogues. In view of its important role in controlling the novel coronavirus epidemic, it is of great clinical significance to study its deuterated products and prodrugs to further improve the metabolism of S-217622 in vivo and expand the therapeutic window.

### CONTENT OF INVENTION

The technical problem to be solved by the invention is the study of the deuterated compound lacking S-217622 in the prior art and the relatively simple structure of its analogs. Therefore, the invention provides a triazine compound, its intermediate, and a preparation method thereof and the use thereof. Based on retaining the effectiveness of SARS-CoV-2, the compound of the invention can significantly prolong the half-life, reduce the demand for dosage, reduce side effects, and expand the range of treatment window. Therefore, the invention has a very good prospect for making drugs for treating diseases related to coronavirus infection.

The invention provides a triazine derivative shown in formula (1) or a pharmaceutically acceptable salt thereof,

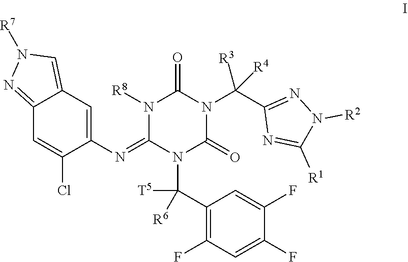

Where R¹ is hydrogen or deuterium,
R² is a methyl group or a methyl group replaced by 1, 2, or 3 tritium,
R³ is hydrogen or deuterium,
R⁴ is hydrogen or deuterium,
R⁵ is hydrogen or deuterium,
R⁶ is hydrogen or deuterium,
R⁷ is a methyl group or a methyl group replaced by 1, 2, or 3 tritium,
R⁸ is hydrogen,

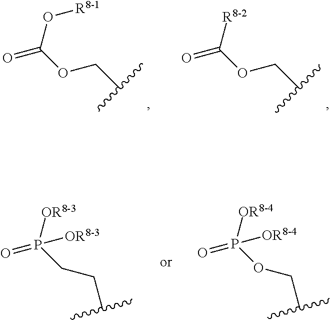

R⁸⁻¹ is C₁-C₁₀ alkyl,
R⁸⁻² is C₁-C₁₀ alkyl,
Each R⁸⁻³ is independently hydrogen, C₁-C₁₀ alkyl, or sodium,
Each R⁸⁻⁴ is independently hydrogen, C₁-C₁₀ alkyl, or sodium,
In addition, R¹, R², R³, R⁴, R⁵, R⁶, and R⁷ contain at least one deuterium.

The invention provides a triazine derivative shown in formula (1′) or a pharmaceutically acceptable salt thereof,

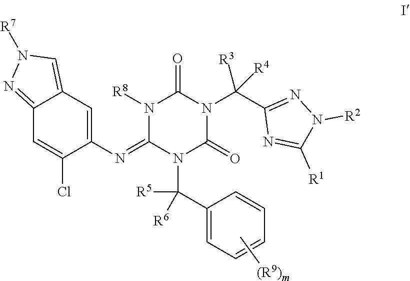

Where R¹ is hydrogen or deuterium,
R² is a methyl group or a methyl group replaced by 1, 2, or 3 tritium,
R³ is hydrogen or deuterium,
R⁴ is hydrogen or deuterium,
R⁵ is hydrogen or deuterium,
R⁶ is hydrogen or deuterium,
R⁷ is a methyl group or a methyl group replaced by 1, 2, or 3 tritium,
R⁸ is hydrogen,

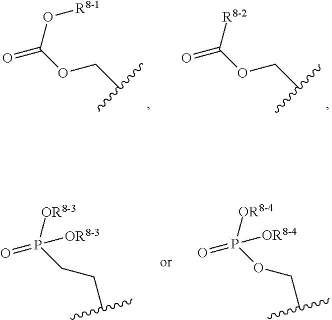

R⁸⁻¹ is C₁-C₁₀ alkyl,
R⁸⁻² is C₁-C₁₀ alkyl,
Each R⁸⁻³ is independently hydrogen, C₁-C₁₀ alkyl, or sodium,
Each R⁸⁻⁴ is independently hydrogen, C₁-C₁₀ alkyl, or sodium,
Each R9 is independently hydrogen or halogen,
m is 2, 3, 4 or 5,

Moreover, the compound shown in formula I′ satisfies one or both of the following conditions:
(1) R¹, R², R³, R⁴, R⁵, R⁶ and R⁷ contain at least one tritium,
(2) R⁸ is

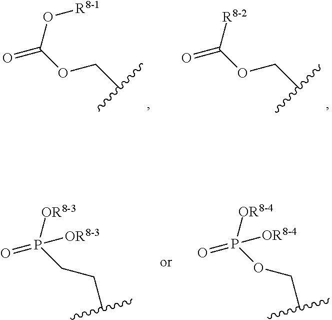

In one scheme, certain substituents in the triazine derivatives shown in formula (I) or their pharmaceutically acceptable salts may be further defined as follows, and substituents not covered below are defined as described in any scheme of the invention (hereinafter referred to as “in a scheme”):

In one scheme, certain substituents in a triazine derivative shown in formula (I′) or in a pharmaceutically acceptable salt thereof may be further defined as follows, and substituents not covered below are defined as described in any scheme of the invention (hereinafter referred to as “in a scheme”):

In one scheme, R⁸⁻¹, the C₁-C₁₀ alkyl may be C₁-C₆ alkyl, preferably methyl, ethyl, n-propyl, isopropyl, n-butyl, isobutyl, sec-butyl, or tert-butyl, such as methyl, ethyl, or isopropyl.

In one scheme, R⁸⁻², the C₁-C₁₀ alkyl may be C₁-C₆ alkyl, preferably methyl, ethyl, n-propyl, isopropyl, n-butyl, isobutyl, sec-butyl, or tert-butyl, such as methyl, ethyl, isopropyl, n-butyl, or tert-butyl.

In a scheme, R⁸⁻³, the C₁-C₁₀ alkyl may be C₁-C₆ alkyl, preferably methyl, ethyl, n-propyl, isopropyl, n-butyl, isobutyl, sec-butyl, or tert-butyl.

In a scheme, R⁸⁻⁴, the C₁-C₁₀ alkyl group may be C₁-C₆ alkyl group, preferably methyl, ethyl, n-propyl, isopropyl, n-butyl, isobutyl, sec-butyl, or tert-butyl.

In a scheme, when R⁸ is hydrogen, the three classes of derivatives shown in formula (I) may also be their tautomers.

For example,

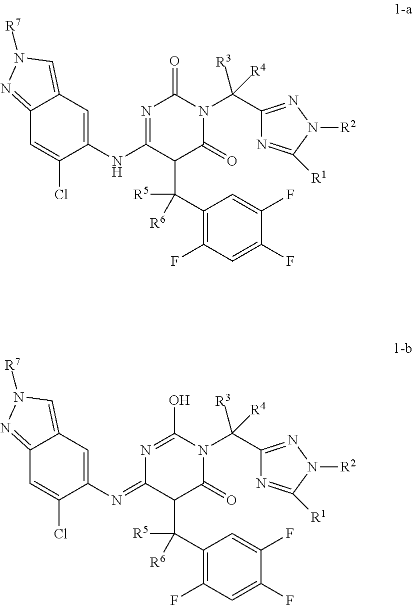

In one scheme, when R⁸ is hydrogen, the triazine derivative shown in the formula (I′) may also be its tautomer, for example

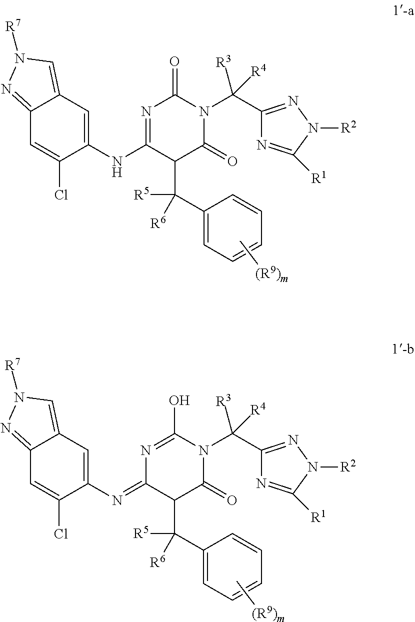

In one scheme, R⁹, the halogen may be fluorine, chlorine, bromine or iodine, such as fluorine.

In one scheme, R² is methyl or —CD₃.

In one scheme, R⁷ is methyl or —CD₃.

In one scheme, R⁸⁻¹ is C₁-C₆ alkyl.

In one scheme, R⁸⁻² is C₁-C₆ alkyl.

In one scheme, R⁸⁻³ is sodium.

In one scheme, R⁸⁻⁴ is sodium.

In one scheme, both R³ and R⁴ are deuterium.

In one scheme, both R⁵ and R⁶ are deuterium.

In one scheme, m is 2 or 3,

In one scheme, R⁹ is fluorine.

In one scheme, R⁸ is hydrogen,

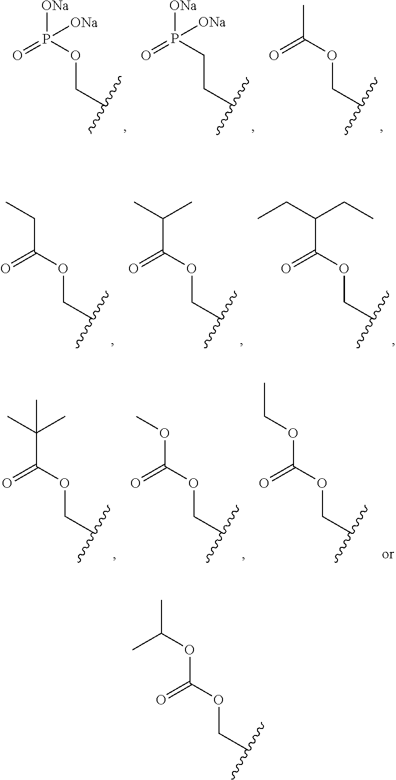

In one scheme,

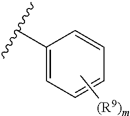

is

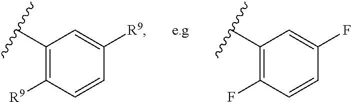

In one scheme,

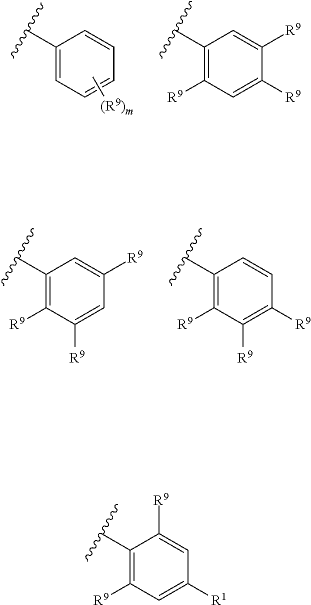

For example

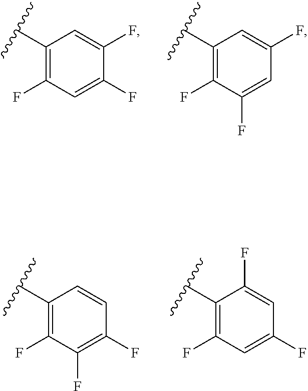 [SMILES: *c1cc(F)c(F)cc1F ; *c1cc(F)cc(F)c1F ; *c1ccc(F)c(F)c1F ; *c1c(F)cc(F)cc1F]

### Better

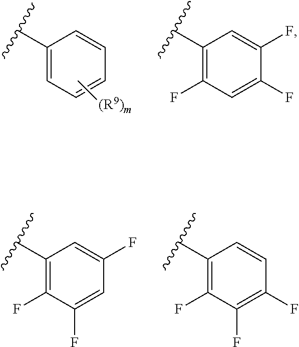

In one scheme, R¹ is deuterium and R² is methyl; R³ is hydrogen; R⁴ is hydrogen; R⁵ is hydrogen; R⁶ is hydrogen; R⁷ is methyl; R⁸ is hydrogen,

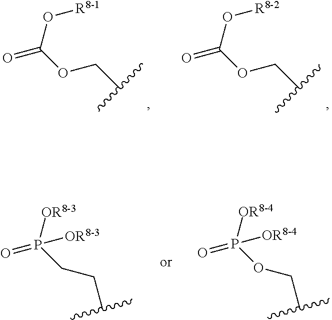

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium.

In one scheme, R¹ is deuterium and R² is methyl; R³ is hydrogen; R⁴ is hydrogen; R⁵ is hydrogen; R⁶ is hydrogen; R⁷ is methyl; R⁸ is hydrogen,

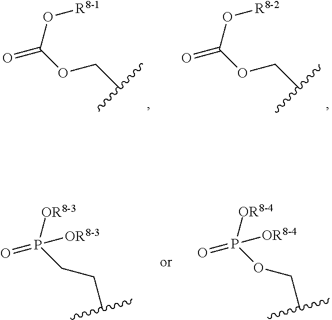

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen:
m is 2, 3, 4, or 5.

In one scheme, R¹ is deuterium and R² is —CD3; R³ is hydrogen; R⁴ is hydrogen; R⁵ is hydrogen; R⁶ is hydrogen; R⁷ is methyl; R⁸ is hydrogen,

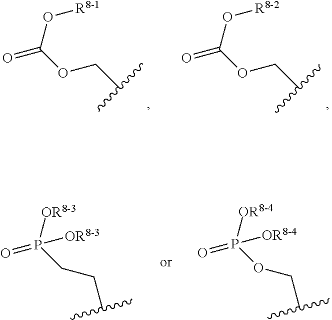

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium.

In one scheme, R¹ is deuterium and R² is —CD₃; R³ is hydrogen; R⁴ is hydrogen; R⁵ is hydrogen; R⁶ is hydrogen; R⁷ is methyl or —CD₃; R⁸ is hydrogen,

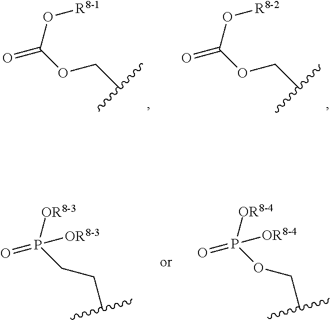

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen:
m is 2, 3, 4, or 5.

Better, in one scheme, R¹ is deuterium and R² is —CD₃; R³ is hydrogen; R⁴ is hydrogen; R⁵ is hydrogen; R⁶ is hydrogen; R⁷ is methyl or —CD₃; R⁸ is hydrogen,

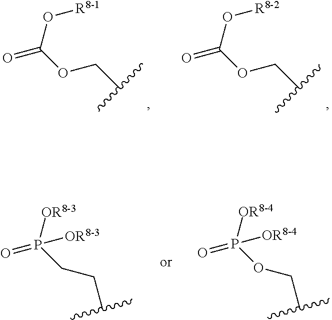

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. R⁹ is fluorine.

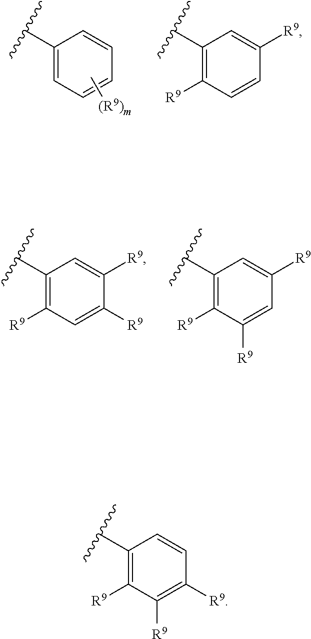

In one scheme, R¹ is hydrogen and R² is methyl; R³ is tritium; R⁴ is tritium; R⁵ is hydrogen; R⁶ is hydrogen; R⁷ is methyl; R⁸ is hydrogen,

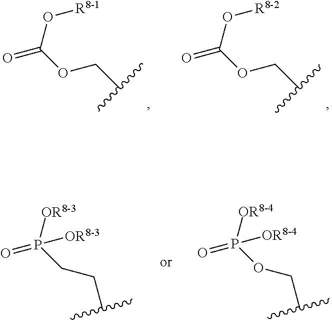

R⁸⁻¹ is C₁-C₆ alkyl, R⁸⁻² is C₁-C₆ alkyl.

Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen. m is 2, 3, 4, or 5.

In one scheme, R¹ is hydrogen and R² is methyl; R³ is hydrogen; R⁴ is hydrogen; R⁵ is tritium; R⁶ is tritium; R⁷ is methyl; R⁸ is hydrogen,

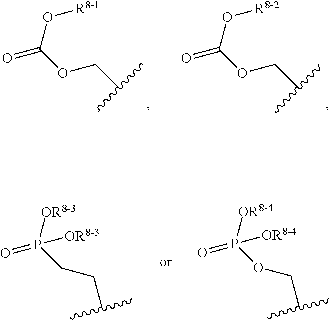

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium.

In one scheme, R¹ is hydrogen and R² is methyl; R³ is hydrogen; R⁴ is hydrogen; R⁵ is tritium; R⁶ is tritium; R⁷ is methyl; R⁸ is hydrogen,

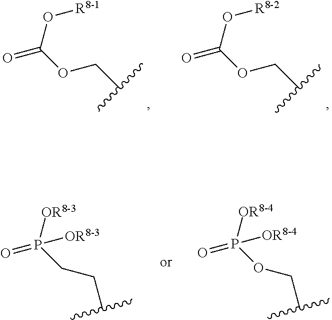

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen, m is 2, 3, 4, or 5.

In one scheme, R¹ is hydrogen and R² is methyl; R³ is hydrogen; R⁴ is hydrogen; R⁵ is hydrogen; R⁶ is hydrogen; R⁷ is —CD₃; R⁸ is hydrogen,

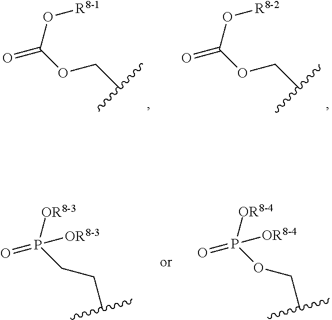

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium.

In one scheme, R¹ is hydrogen and R² is methyl or —CD₃; R³ is hydrogen; R⁴ is hydrogen; R⁵ is hydrogen; R⁶ is hydrogen; R⁷ is —CD₃; R⁸ is hydrogen,

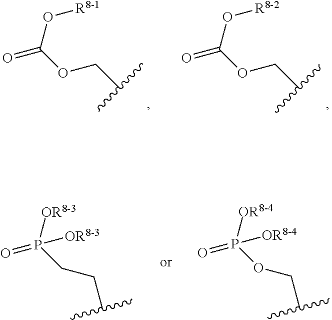

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen, m is 2, 3, 4, or 5.

In one scheme, R¹ is hydrogen or deuterium, R² is methyl or —CD₃; R³ is hydrogen or deuterium; R⁴ is hydrogen or deuterium; R⁵ is hydrogen or deuterium; R⁶ is hydrogen or deuterium: R⁷ is methyl or —CD₃; R⁸ is

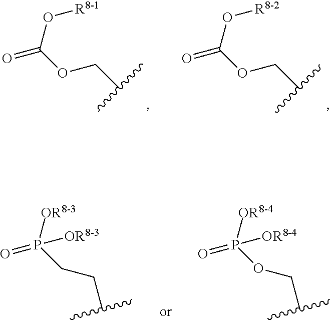

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen, m is 2, 3, 4, or 5.

Better, in one scheme, R¹ is hydrogen or deuterium, R² is methyl or —CD₃; R³ is hydrogen or deuterium; R⁴ is hydrogen or deuterium; R⁵ is hydrogen or deuterium; R⁶ is hydrogen or deuterium; R⁷ is methyl or —CD₃; R⁸ is

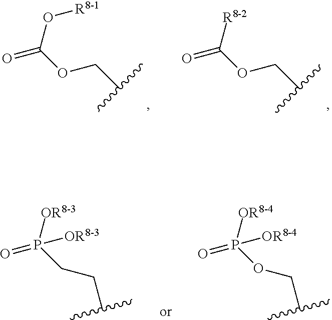

R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium, R⁹ is fluorine,

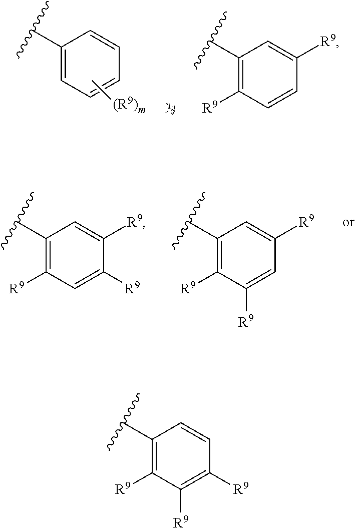

In a scheme, the triazine derivative shown in the formula (I′) is any of the following compounds:

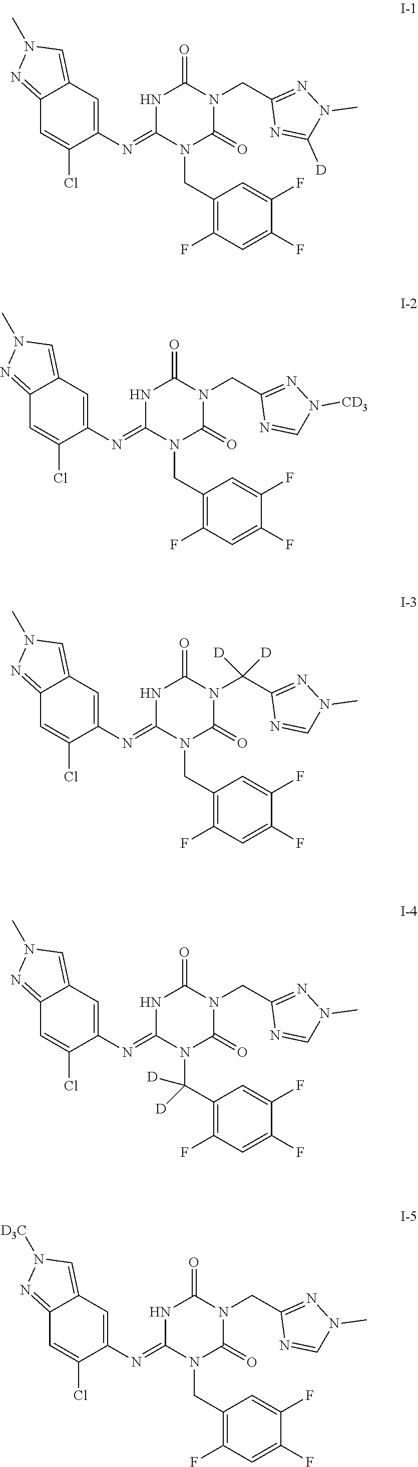

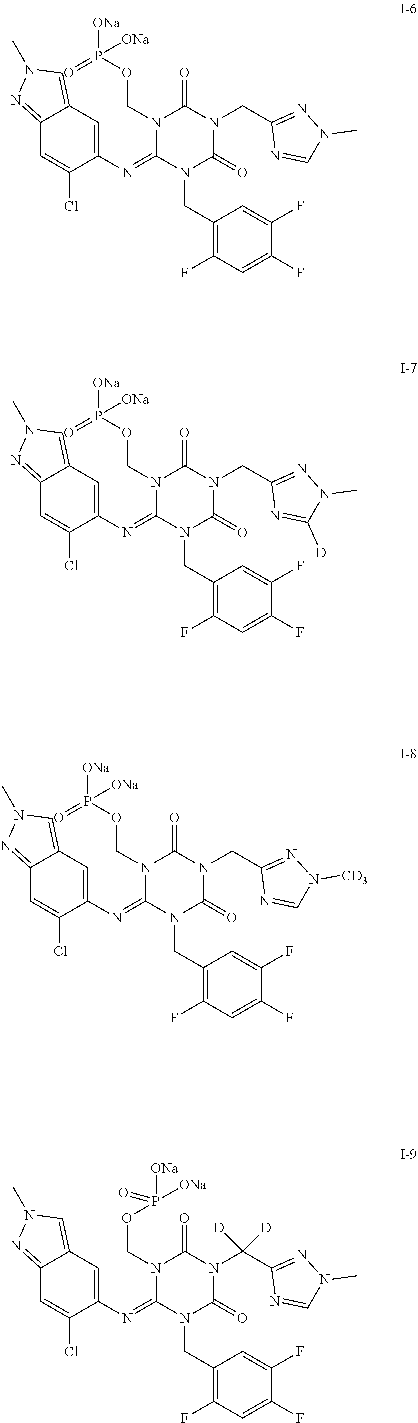

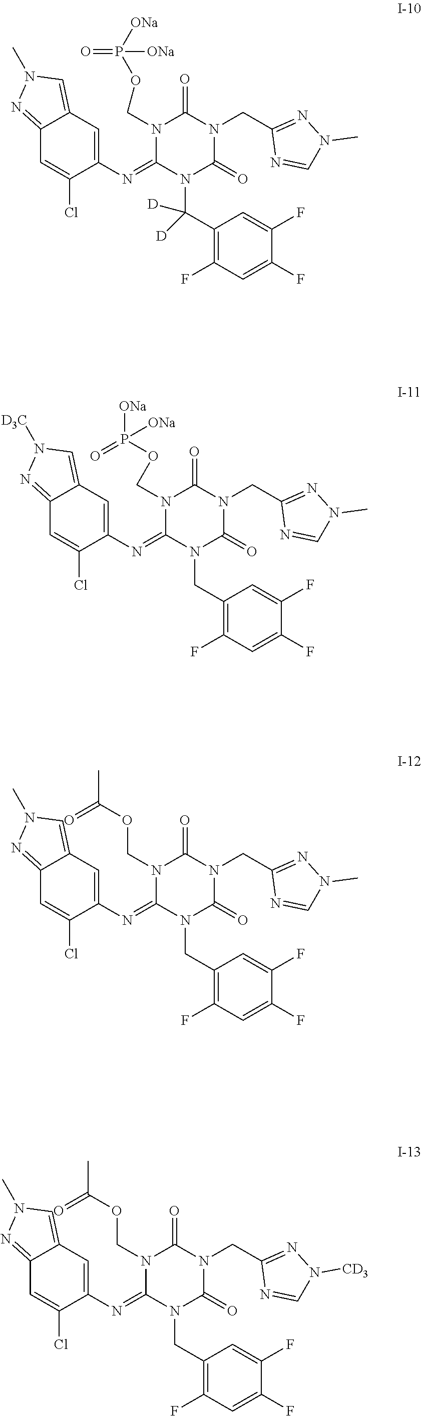

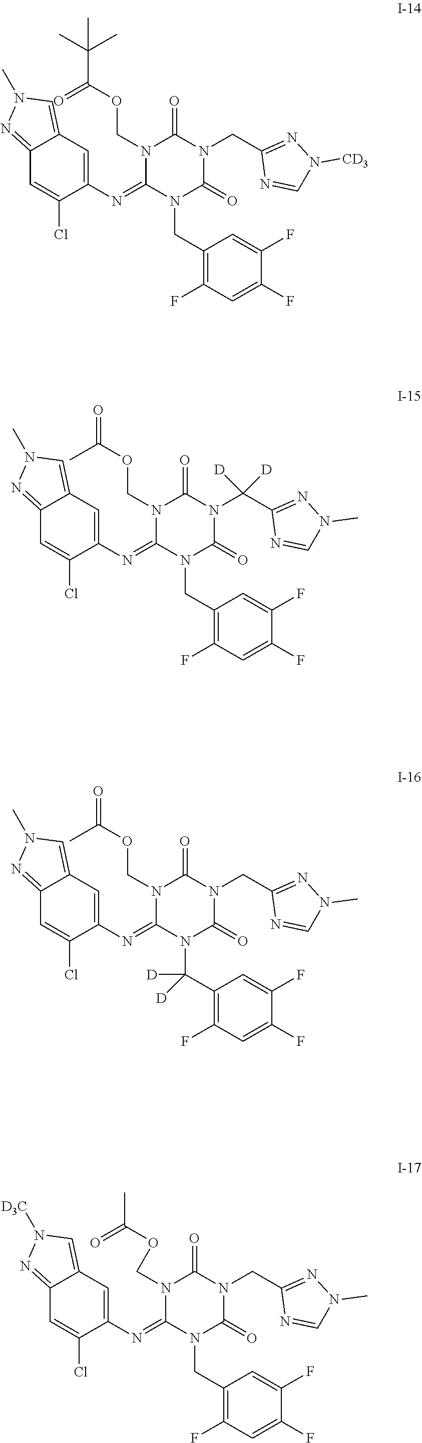

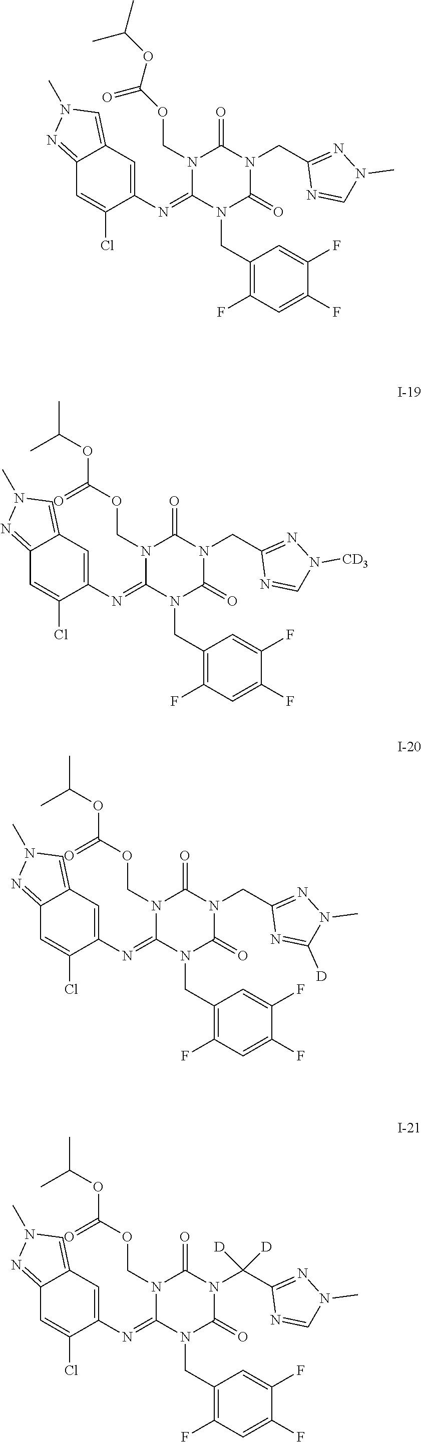

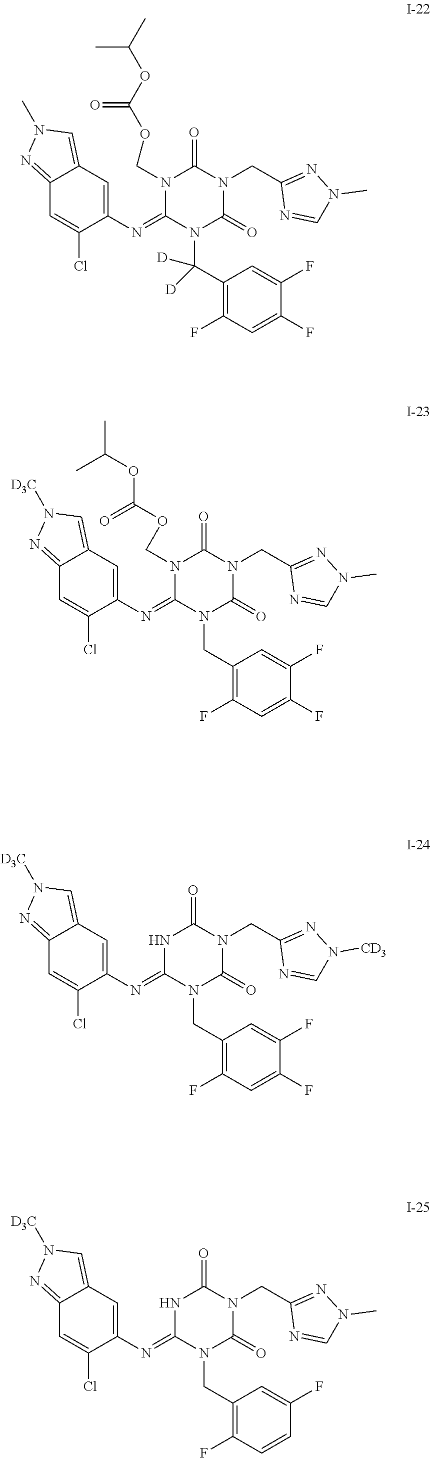

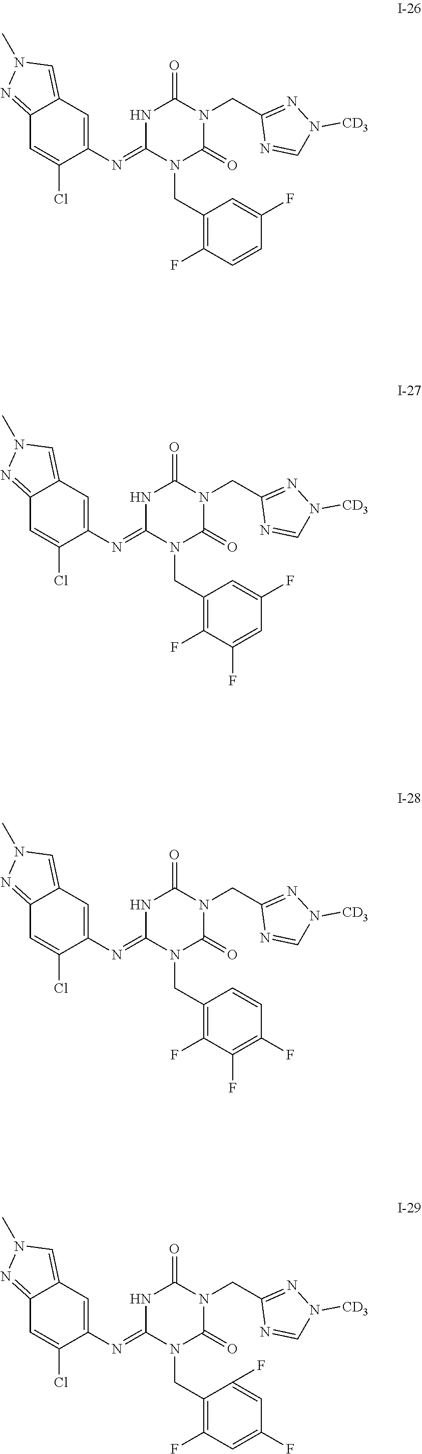 [SMILES: [2H]C([2H])([2H])n1cnc(Cn2c(=O)[nH]/c(=N\c3cc4cn(C)nc4cc3Cl)n(Cc3cc(F)ccc3F)c2=O)n1 ; [2H]C([2H])([2H])n1cnc(Cn2c(=O)[nH]/c(=N\c3cc4cn(C)nc4cc3Cl)n(Cc3cc(F)cc(F)c3F)c2=O)n1 ; [2H]C([2H])([2H])n1cnc(Cn2c(=O)[nH]/c(=N\c3cc4cn(C)nc4cc3Cl)n(Cc3ccc(F)c(F)c3F)c2=O)n1 ; [2H]C([2H])([2H])n1cnc(Cn2c(=O)[nH]/c(=N\c3cc4cn(C)nc4cc3Cl)n(Cc3c(F)cc(F)cc3F)c2=O)n1]

or

The invention also provides a triazine derivative or a pharmacologically acceptable salt as shown in formula (III).

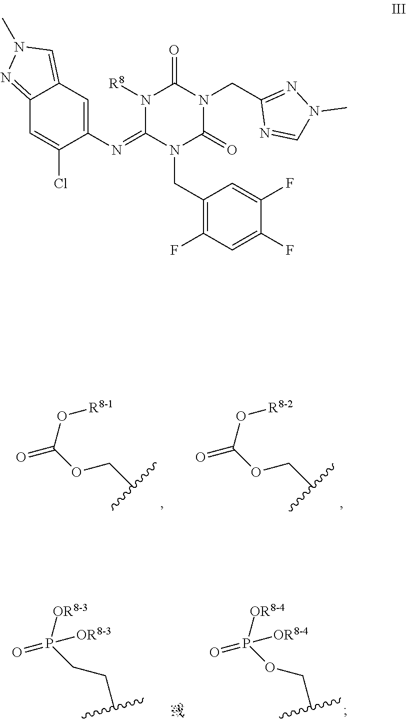

R⁸ is hydrogen,
R⁸⁻¹ is C₁-C₁₀ alkyl,
R⁸⁻² is C₁-C₁₀ alkyl,
Each R⁸⁻³ is independently hydrogen, C₁-C₁₀ alkyl, or sodium,
Each R⁸⁻⁴ is independently hydrogen, C₁-C₁₀ alkyl, or sodium,
formula (III) is not

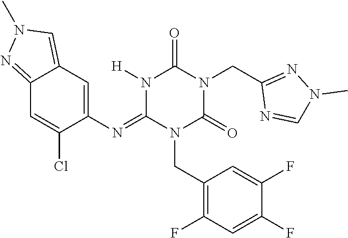

In one scheme, certain substituents in a deuterotriazine derivative shown in formula (III) or in a pharmaceutically acceptable salt thereof may be further defined as follows, and substituents not covered below are defined as described in any scheme of the invention (hereinafter referred to as “in one scheme”). In R⁸⁻¹, the C₁˜C₁₀ alkyl may be C₁˜C₆ alkyl, preferably methyl, ethyl, n-propyl, isopropyl, n-butyl, isobutyl, sec-butyl or tert-butyl, such as methyl, ethyl or isopropyl.

In one scheme, R⁸⁻², the C₁˜C₁₀ alkyl group may be C₁˜C₆ alkyl groups, preferably methyl, ethyl, n-propyl, isopropyl, n-butyl, isobutyl, sec-butyl or tert-butyl groups, such as methyl, ethyl, isopropyl, n-butyl or tert-butyl groups.

In a scheme, R⁸⁻³, the C₁˜C₁₀ alkyl group may be C₁˜C₆ alkyl group, preferably methyl, ethyl, n-propyl, isopropyl, n-butyl, isobutyl, sec-butyl, or tert-butyl.

In a scheme, R⁸⁻⁴, the C₁˜C₁₀ alkyl group may be C₁˜C₆ alkyl group, preferably methyl, ethyl, n-propyl, isopropyl, n-butyl, isobutyl, sec-butyl, or tert-butyl.

In one scheme, R⁸⁻¹ is C₁˜C₆ alkyl.

In one scheme, R⁸⁻² is C₁˜C₆ alkyl.

In one scenario, R⁸⁻³ is sodium.

In one scenario, R⁸⁻⁴ is sodium.

In one scheme, when R⁸ is hydrogen, the triazine derivative shown in the formula (III) may also be its tautomer, e.g.

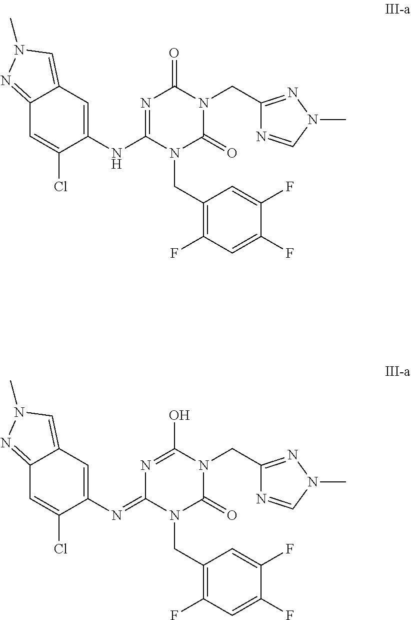 [SMILES: Cn1cnc(Cn2c(=O)nc(Nc3cc4cn(C)nc4cc3Cl)n(Cc3cc(F)c(F)cc3F)c2=O)n1 ; Cn1cnc(Cn2c(O)n/c(=N\c3cc4cn(C)nc4cc3Cl)n(Cc3cc(F)c(F)cc3F)c2=O)n1]

In one scheme, R⁸ is hydrogen,

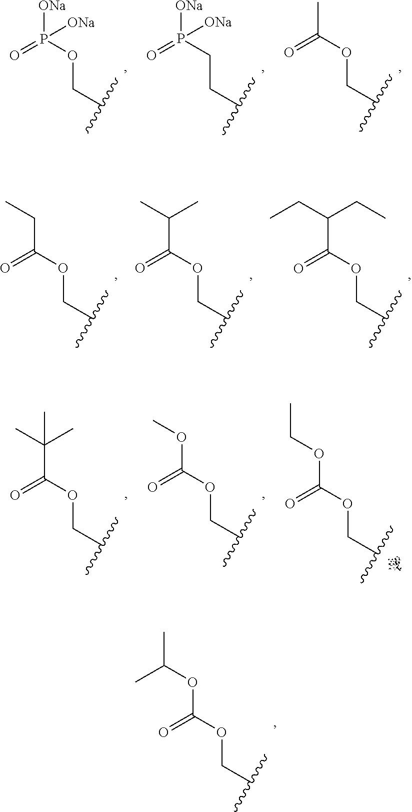

The invention also provides a triazine derivative shown in formula VI or a pharmaceutically acceptable salt thereof.

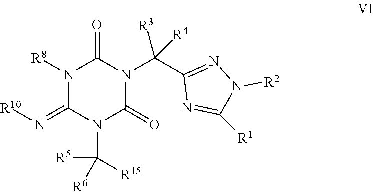

Where R¹ is hydrogen or deuterium.
R² is a methyl group or a methyl group replaced by 1, 2 or 3 deuterium.
R³ for hydrogen or deuterium.
R⁴ is hydrogen or deuterium.
R⁵ for hydrogen or deuterium.
R⁶ is hydrogen or deuterium.
R⁸ is hydrogen,

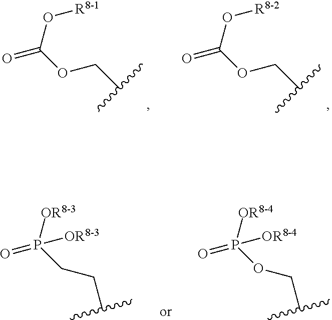

R⁸⁻¹ is C₁˜C₁₀ alkyl.
R⁸⁻² is C₁-C₁₀ alkyl.
Each R⁸⁻³ is independently hydrogen, C₁-C₁₀ alkyl, or sodium.
Each R⁸⁻⁴ is independently hydrogen, C₁˜C₁₀ alkyl, or sodium.
R¹⁰ is a 6-10 meta heteroaryl group that is not replaced or is replaced by one or more R¹⁰⁻¹; The type of heteroatom of the 6-10 meta heteroaryl group is selected from one or more of N, O and S, and the number of heteroatom is 1, 2 or 3;
Each R¹⁰⁻¹ is independently a C₁-C₆ alkyl, halogen, or deuterium that is not replaced or is replaced by one or more R¹⁰⁻¹⁻¹.
Each R¹⁰⁻¹⁻¹ is independently a halogen or deuterium.

### R¹⁵ is

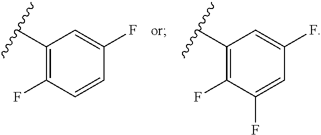 [SMILES: *c1cc(F)ccc1F ; *c1cc(F)cc(F)c1F]

In a scheme, certain substituents in the triazine derivatives or pharmaceutically acceptable salts thereof shown in formula VI may be further defined as follows, and substituents not covered below are defined as described in any scheme of the invention (hereinafter referred to as “in one scheme”);

In R⁸⁻², the C₁-C₁₀ alkyl may be C₁-C₆ alkyl, preferably methyl, ethyl, n-propyl, isopropyl, n-butyl, isobutyl, sec-butyl, or tert-butyl, such as methyl, ethyl, isopropyl, n-butyl, or tert-butyl.

In a certain scheme, R⁸⁻³, the C₁˜C₁₀alkyl group may be C₁˜C₆. Alkyl, preferably methyl, ethyl, n-propyl, isopropyl, n-butyl, isobutyl, sec-butyl, or tert-butyl.

In a scheme, R⁸⁻⁴, the C₁˜C₁₀ alkyl group may be C₁-C₆ alkyl group, preferably methyl, ethyl, n-propyl, isopropyl, n-butyl, isobutyl, sec-butyl, or tert-butyl.

In a scheme, R¹⁰, the 6-10 meta heteroaryl group may be 5 and 6 meta heteroaryl group, and/or the 6-10 meta heteroaryl group may have a heteroatom class of N and/or O, and/or the 6-10 meta heteroaryl group may have a heteroatom number of 2; The 6-10 meta heteraryl group is preferably

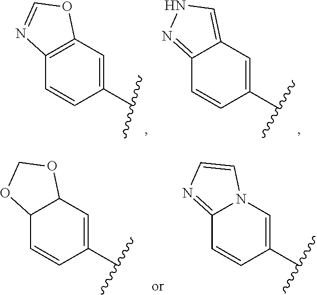 [SMILES: *c1ccc2ncoc2c1 ; *c1ccc2n[nH]cc2c1 ; *C1=CC2OCOC2C=C1 ; *c1ccc2nccn2c1]

In one scheme, R¹⁰⁻¹, the C₁-C₆ alkyl group may be methyl, ethyl, n-propyl, isopropyl, n-butyl, isobutyl, sec-butyl, or tert-butyl, such as methyl.

In one scheme, R¹⁰⁻¹, the halogen may be fluorine, chlorine, bromine or iodine, such as fluorine or chlorine.

In one scheme, R¹⁰⁻¹⁻¹ the halogen may be a atmosphere, chlorine, bromine or iodine, such as fluorine or chlorine.

In one scheme, R¹ is hydrogen.

In one scheme, R² is methyl or —CD3.

In one scenario, R³ is hydrogen.

In one scenario, R⁴ is hydrogen.

In one scenario, R⁵ is hydrogen.

In one scenario, R⁶ is hydrogen.

In a scheme, each R¹⁰⁻¹ is independently chlorine, methyl, fluorine, or —CD₃;

In a scheme, R10, the 6-10 meta heteroaryl group is

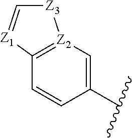

Z1 is N or O; Z2 is N or C; Z3 is C or O; In a better way, R¹⁰ is

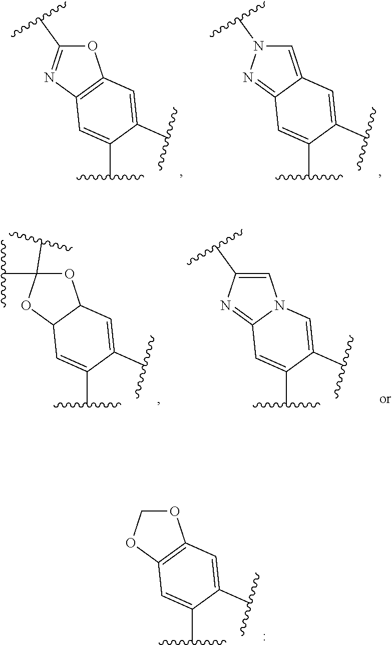 [SMILES: *c1nc2cc(*)c(*)cc2o1 ; *c1cc2cn(*)nc2cc1* ; *C1=CC2OC(*)(*)OC2C=C1* ; *c1cn2cc(*)c(*)cc2n1 ; *c1cc2c(cc1*)OCO2]

Another example

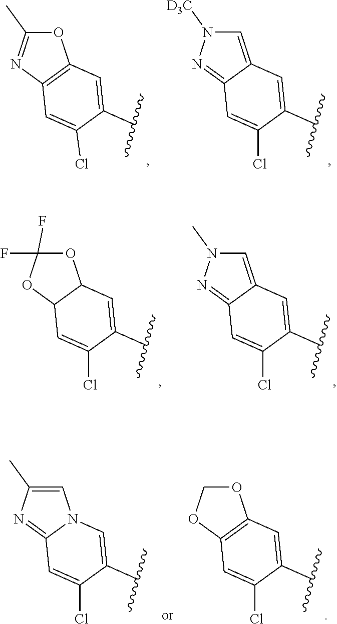 [SMILES: *c1cc2oc(C)nc2cc1Cl ; *c1cc2cn(C([2H])([2H])[2H])nc2cc1Cl ; *C1=CC2OC(F)(F)OC2C=C1Cl ; *c1cc2cn(C)nc2cc1Cl ; *c1cn2cc(C)nc2cc1Cl ; *c1cc2c(cc1Cl)OCO2]

The invention also provides a preparation method of a triazine derivative as shown in formula (I′) above, which is method 1, method 2, method 3, method 4 or method 5.

The method 1 comprises the following steps where, in a solvent (e.g., anhydrous tetrahydrofuran), in the presence of a base (e.g., hexamethyldisilylamine lithium), a compound shown in formula I′-S1 reacts with heavy water as shown below to obtain a compound shown in formula I′.

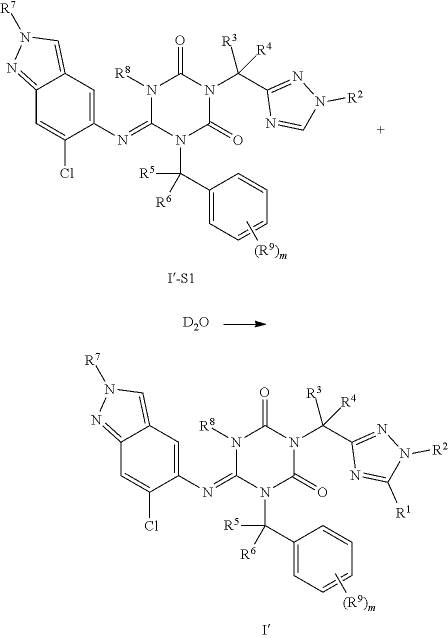

Where R¹ is deuterium; R^{Z}, R³, R⁴, R⁵, R⁶, R⁷ and R⁸ are defined as described before.

Method 2 consists of the following steps: In a solvent (e.g., a mixed solution of acetic acid and tert-butanol), a compound shown in formula I′-S2 reacts with a compound shown in formula I′-S3 as shown below to obtain a compound shown in formula I′.

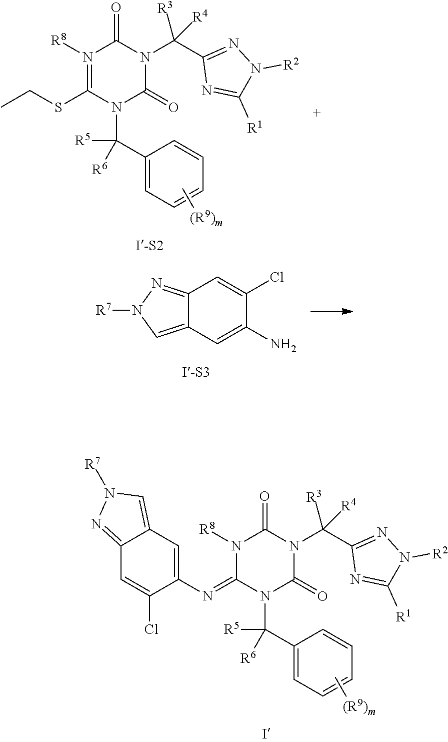

Where R¹, R², R³, R⁴, R⁵, R⁶, R⁷ and R⁸ are defined as described above;

Method 3 consists of the following steps in which, in a solvent (e.g., N, N-dimethylformamide or dimethylacetamide) and in the presence of a base (e.g., anhydrous cesium carbonate or potassium

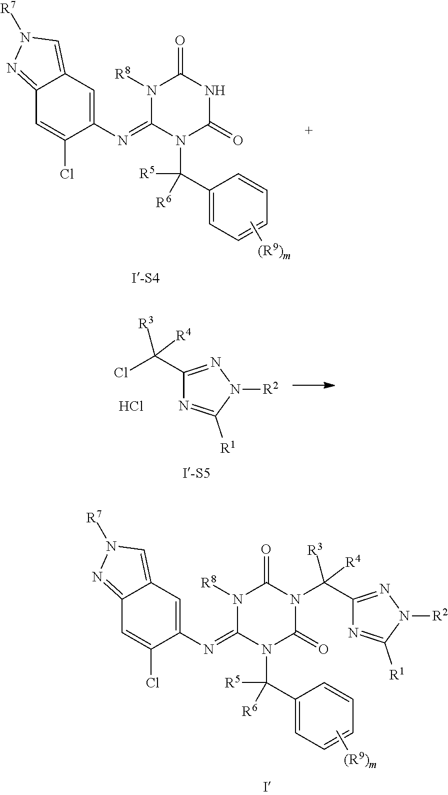

carbonate), a compound shown in formula I′-S4 reacts with a compound shown in formula I′-S5 as shown below to obtain a compound shown in formula I′

Where R¹, R², R³, R⁴, R⁵, R⁶, R⁷ and R⁸ are defined as described above.

The method 4 consists of the following steps, in a solvent (e.g., dichloromethane), a compound shown in formula I′-S6 reacts with sodium hydroxide (e.g., an ethanol solution of sodium hydroxide) as shown below to obtain a compound shown in formula I′

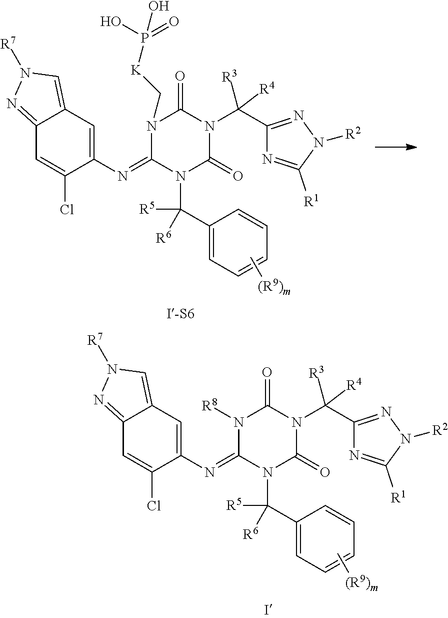

Where K is or CH₂, R⁸ is

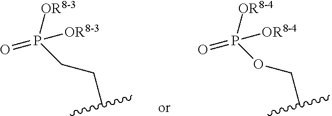

Each R⁸⁻³ and each R⁸⁻⁴ are sodium, Where R¹, R², R³, R⁴, R⁵, R⁶, R⁷ are defined as described above.

Method 5 consists of the following steps: In a solvent (e.g., N, N-dimethylformamide or dimethylacetamide), in the presence of a base (e.g., sodium hydride), a compound shown in formula I′-S7 reacts with a compound shown in formula I′-S8 as shown below to obtain a compound shown in formula I′.

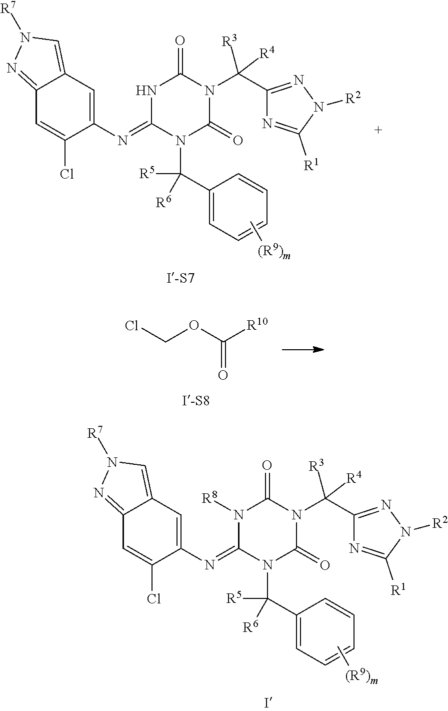

Where R¹⁰ is C₁˜C₁₀ alkyl or —OC₁˜C₁₀ alkyl, R⁸ is

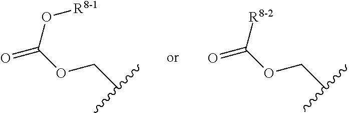

Each R⁸⁻¹ and each R⁸⁻² are independently C₁˜C₁₀ alkyl. R¹, R², R³, R⁴, R⁵, R⁶, R⁷ are defined as described above.

Methods 1 to 5 may be conventional methods in this field, and the preparation conditions and operations may be conventional conditions and operations for this type of reaction in this field.

The invention also provides a binary co-product formed by a triazine derivative and an acid as shown in formula (I), (III) or (VI) above; The acids are malic acid, maleic acid, citric acid, ascorbic acid, mandelic acid, tartaric acid, fumaric acid, preferably fumaric acid.

The invention also provides a binary eutectic of a triazine derivative formed with an acid as shown in formula (I′), (D), (III) or (VI) above; The acids are acetic acid, malic acid, maleic acid, citric acid, ascorbic acid, mandelic acid, tartaric acid, fumaric acid or acetic acid, preferably acetic acid or fumaric acid.

In a scheme, the binary co-goods are

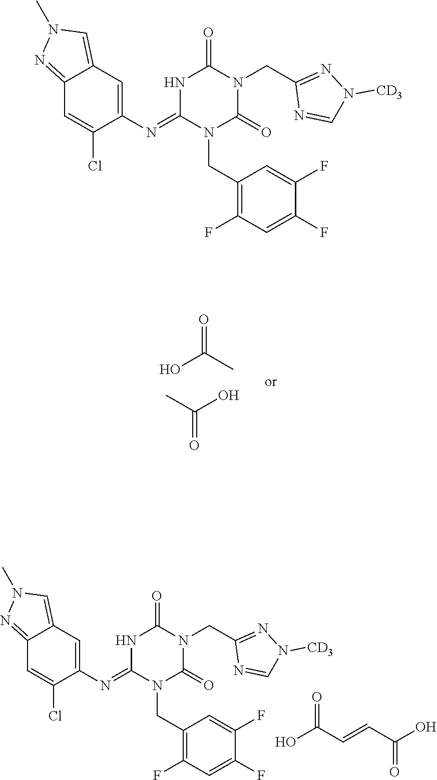

In a scheme, the binary co-goods are

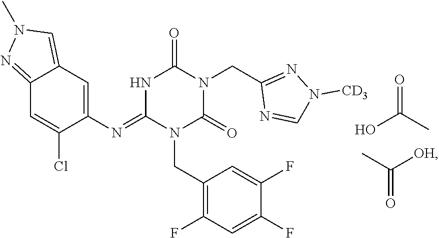

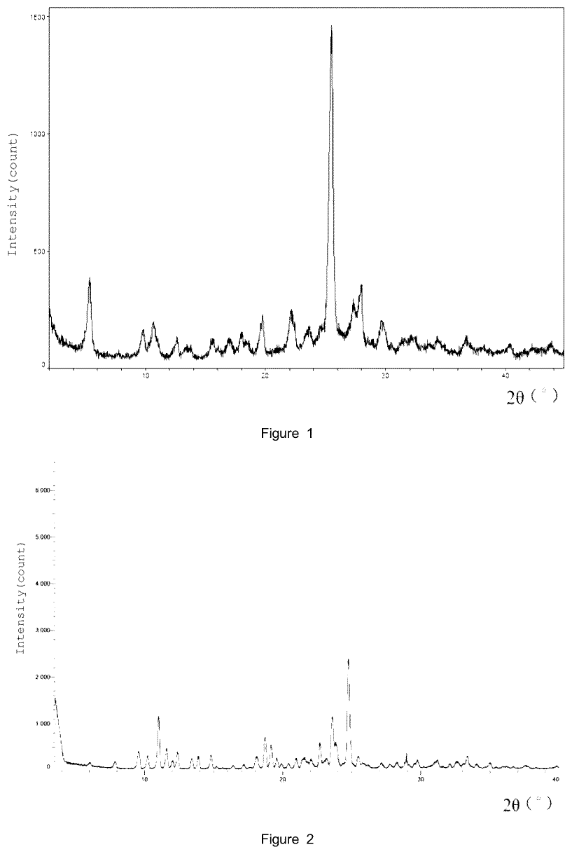

Its X-ray powder diffraction pattern represented by 2θ angles has diffraction peaks at 5.4±0.2, 19.8±0.2, 22.2±0.2 and 25.5±0.2. Preferably, the X-ray powder diffraction pattern expressed at 2θ angles also has diffraction peaks at one or more of 9.9±0.2, 10.7±0.2, 12.6±0.2, 27.3±0.2, and 28.0±0.2. Preferably, its X-ray powder diffraction pattern expressed at 2θ angles is shown in FIG. 1.

In a scheme, the binary co-goods are

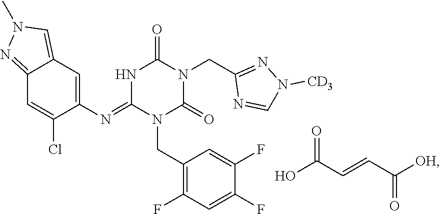

Its X-ray powder diffraction pattern represented by 2θ angles has diffraction peaks at 7.85±0.2°, 9.55±0.2°, 10.22±0.2°, 12.01±0.2°, 13.88±0.2°, 14.79±0.2°, 17.19±0.2°, 18.71±0.2° 19.16±0.2°, 23.60±0.2°, 23.85±0.2°, 24.76±0.2°, and 28.95±0.2°. X-ray powder diffraction pattern represented by 2θ angles also has diffraction peaks at one or more places of 6.05±0.2°, 11.02±0.2°, 11.60±0.2°, 12.39±0.2°, 13.43±0.2°, 15.21±0.2°, 16.41±0.2°, 18.13±0.2°, 19.59±0.2°, 19.91±0.2°, 20.43±0.2°, 20.97±0.2°, 21.53±0.2°, 22.04±0.2°, 22.70±0.2°, 23.16±0.2°, 25.47±0.2°, 27.13±0.2°, 27.77±0.2°, 28.28±0.2°, 29.76±0.2°, 31.17±0.2°, 32.11±0.2°, 32.64±0.2°, 33.34±0.2°, 34.03±0.2°, and 35.00±0.2° Preferably, its X-ray powder diffraction pattern expressed at 2θ angles is shown in FIG. 2.

The invention also provides a method for preparing a binary coproduct of a triazine derivative with an acid as described in formula (I′), (I), (III) or (VI), which includes the following steps to react a triazine derivative as described in formula (I) with an acid. A binary coproduct of a triazine derivative and an acid shown in the formula (I′), (I), (III), or (VI) is obtained: the acid is defined as described above.

In a scheme, when the acid is fumaric acid, the preparation method of the triazine derivative shown in formula (I′), (I), (III) or (VI) as a binary coproduct of the acid with the triazine derivative shown in formula (I), (I), (III) or (VI) may include the following steps, in a solvent, to react with fumaric acid, as described above. The triazine derivative shown in formula (I′), (I), (III) or (VI) is obtained as a binary co-product with fumaric acid.

In a scheme, the solvent may be ethyl acetate.

The preparation method of binary coproducts of triazine derivatives and acids shown in the formula (I′), (I), (III) or (VI) may be the conventional method in the field, and the preparation conditions and operations may be the conventional conditions and operations of such reactions in the field.

The invention also provides a triazine derivative shown in formula (I), (III) or (VI) to form a ternary coproduct with nicotinamide and acid: the acids are malic acid, maleic acid, citric acid, ascorbic acid, mandelic acid, tartaric acid, fumaric acid, preferably fumaric acid.

The invention also provides a ternary eutectic formed by triazine derivatives and niacinamide and acid as shown in formula (I′), (I), (III) or (VI) above. The acids are malic acid, maleic acid, citric acid, ascorbic acid, mandelic acid, tartaric acid, fumaric acid or acetic acid, preferably fumaric acid.

In one scheme, the ternary eutectic is,

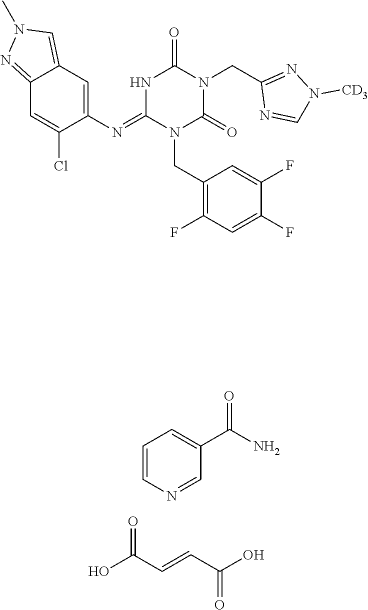

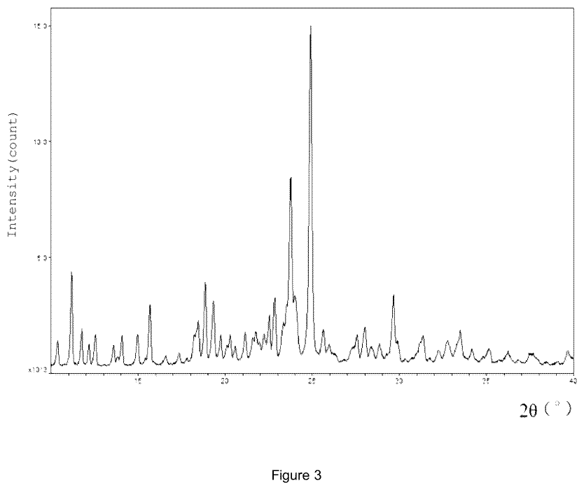

its X-ray powder diffraction pattern represented by 2θ angles has diffraction peaks at 10.406, 11.188, 11.772, 12.202, 12.556, 13.589, 14.075, 14.973, 15.692, 17.37, 18.212, 18.464, 18.874, 19.34, 19.752, 20.104, 20.28, 20.59, 21.154, 21.743, 22.246, 22.536, 22.868, 23.342, 23.767, 24.003, 24.92, 25.641, 25.99, 27.589, 28.038, 28.859, 29.677, 29.911, 31.356, 32.779, 33.502, 35.14, 36.251 and 39.648±0.2. Preferably, its X-ray powder diffraction pattern expressed at 2θ angles is shown in FIG. 3.

In a scheme, the ternary eutectic is,

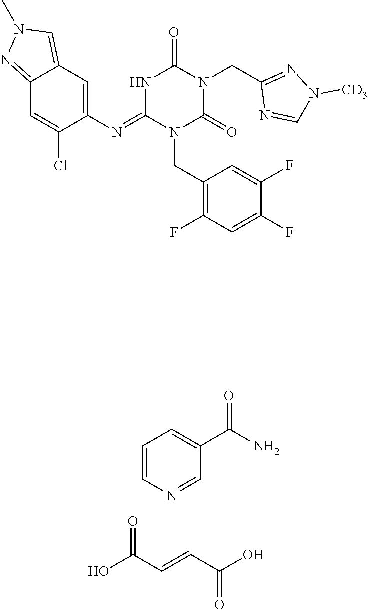

its X-ray powder diffraction pattern represented by 2θ angles has diffraction peaks at 11.2±0.2°, 15.7±0.2°, 18.9±0.2°, 19.3±0.2°, 22.9±0.2°, 24.0±0.2°, 24.9±0.2° and 29.6±0.2°; Preferably, It also has diffraction peaks at one or more places of 10.4±0.2°, 11.8±0.2°, 12.2±0.2°, 12.6±0.2°, 13.6±0.2°, 14.1±0.2°, 15.0±0.2°, 17.4±0.2°, 18.2±0.2°, 18.5±0.2° 19.8±0.2°, 20.1±0.2°, 20.3±0.2°, 20.6±0.2°, 21.2±0.2°, 21.7±0.2°, 22.2±0.2°, 22.5±0.2°, 23.3±0.2°, 23.8±0.2°, 25.6±0.2°, 26.0±0.2°, 27.6±0.2° 28.0±0.2°, 28.9±0.2°, 29.7±0.2°, 29.9±0.2°, 31.4±0.2°, 32.8±0.2°, 33.5±0.2°, 35.1±0.2°, 36.3±0.2° and 39.6±0.2°. Preferably, its X-ray powder diffraction pattern expressed at 2θ angles is shown in FIG. 3.

The invention also provides a method for preparing ternary coproducts of triazine derivatives shown in formula (I′), (I), (III) or (VI) with niacinamide and acid as described above, which includes the following steps to react triazine derivatives, niacinamide and acid as described in formula (I′). To obtain a ternary coproduct of the triazine derivatives shown in the formula (I′), (I), (III) or (VI) with niacinamide and acid; The acid is defined as described above.

In a scheme, the preparation method of the triazine derivative shown in formula (I′), (I), (II) or (VI) with the ternary eutectic of niacinamide and acid may consist of the following steps, reaction in a solvent of the triazine derivative, niacinamide and fumaric acid shown in formula (I), (I), (III) or (VI), as described above. The triazine derivatives shown in formula (I′), (I), (III) or (VI) are obtained as ternary coproducts with niacinamide and acid.

In a scheme, the solvent can be ethyl acetate.

The triazine derivatives shown in formula (I′), (I), (III) or (VI) to form ternary coproducts with niacinamide and acid may be conventional methods in this field, and the preparation conditions and operations may be conventional conditions and operations for such reactions in this field.

The invention also provides a pharmaceutical composition comprising substance B and one or more pharmaceutically acceptable carriers; The substance B is a triazine derivative or a pharmaceutically acceptable salt thereof as indicated in formula (I), (III) or VI, or a binary eutectic or ternary eutectic as indicated previously. In the pharmaceutical composition, the amount of substance B may be a therapeutic effective amount.

The invention also provides a pharmaceutical composition comprising substance B′ and one or more pharmaceutically acceptable carriers: the substance B′ is a triazine derivative or a pharmaceutically acceptable salt thereof as indicated in formula (I′), (I), (III) or (VI), or a binary or ternary eutectic as described above. In the pharmaceutical composition, the amount of the substance B′ may be a therapeutic effective amount.

Pharmaceutical compositions of the present invention may be prepared according to the disclosed contents using any method known to a person skilled in the art. For example, conventional mixing, dissolution, granulation, emulsification, grinding, encapsulation, embedding or freeze-drying processes.

The invention also provides a use of substance B in the preparation of antiviral drugs such as a triazine derivative or a pharmaceutically acceptable salt thereof as indicated in formula (I), (III) or VI above, or a binary or ternary co-product as described above; The viruses are coronaviruses, influenza viruses, respiratory syncytial viruses, flaviviridae viruses, filoviridae viruses, or porcine Epidemic diarrhea virus (PEDV).

The invention also provides a use of substance B′ in the preparation of antiviral drugs, such as a triazine derivative or a pharmaceutically acceptable salt thereof as described in formula (I′), (I), (III) or (VI), such as a binary or ternary co-product or a pharmaceutical composition as described above; The viruses are coronaviruses, influenza viruses, respiratory syncytial viruses, flaviviridae viruses, filoviridae viruses, or porcine Epidemic diarrhea virus (PEDV).

Preferably, the coronavirus is selected from one or more of MERS-CoV, SARS-CoV, and SARS-CoV-2, and the coronavirus is preferred as SARS-CoV-2.

The invention also provides an application of substance B in the preparation of drugs for the treatment and/or prevention of coronavirus-related diseases, in the form of triazine derivatives or pharmaceutically acceptable salts thereof as indicated in formula (I), (III) or VI above, or in the form of binary or ternary co-products as described above.

The invention also provides an application of substance B′ in the preparation of drugs for the treatment and/or prevention of coronavirus-related diseases, in the form of triazine derivatives or pharmaceutically acceptable salts thereof as described in formula (I′), (I), (III) or (VI), as described previously as binary or ternary co-products or as described previously as pharmaceutical compositions.

The invention also provides a pharmaceutical composition comprising substance A and one or more pharmaceutically acceptable carriers; Substance A is a triazine derivative or a pharmaceutically acceptable salt thereof as indicated in formula (I) or (III) above. In the pharmaceutical composition, the amount of the triazine derivative shown in formula (I) or (III), or the pharmaceutically acceptable salt thereof, may be a therapeutic effective amount.

The invention also provides A pharmaceutical composition comprising a substance A′ and one or more pharmaceutically acceptable carriers; The substance A′ is a triazine derivative or a pharmaceutically acceptable salt thereof as indicated in formula (I′), (I), (III) or (VI) above. In the pharmaceutical composition, the amount of the triazine derivative shown in formula (I′), (I), (III) or (VI), or the pharmaceutically acceptable salt thereof, may be a therapeutic effective amount.

The pharmaceutically acceptable carriers (pharmaceutical excipients) may be those excipients widely used in the field of pharmaceutical production. Excipients are primarily intended to provide a safe, stable, and functional pharmaceutical composition, and may also provide a method for dissolution of the active ingredient at the desired rate after the subject receives the administration of the composition, or to facilitate the effective absorption of the active ingredient after the subject receives the administration of the composition. The pharmaceutical excipients may be inert fillers or provide a function, such as stabilizing the overall pH of the composition or preventing degradation of the active component of the composition. The pharmaceutical excipients may include one or more of the following excipients. Adhesives, suspension AIDS, emulsifiers, thinners, fillers, granulants, adhesives, disintegrants, lubricants, anti-adhesive agents, flow AIDS, wetting agents, gelling agents, absorption delay agents, dissolution inhibitors, enhancers, absorbents, buffers, chelators, preservatives, colorants, taste correction agents and sweeteners.

Pharmaceutical compositions of the present invention may be prepared according to the disclosed contents using any method known to a person skilled in the art. For example, conventional mixing, dissolution, granulation, emulsification, grinding, encapsulation, embedding or freeze-drying processes.

The invention also provides a substance A in the preparation of antiviral drugs, which is a triazine derivative or a pharmaceutically acceptable salt thereof as indicated in formula (I) or (III), or a composition as described above; The viruses are coronaviruses, influenza viruses, respiratory syncytial viruses, flaviviridae viruses, filoviridae viruses, or porcine Epidemic diarrhea virus (PEDV).

The invention also provides a substance A′ in the preparation of an antiviral drug, which is a triazine derivative or a pharmaceutically acceptable salt thereof as indicated in formula (I′), (I), (III) or (VI), or a composition as described above. The viruses are coronaviruses, influenza viruses, respiratory syncytial viruses, flaviviridae viruses, filoviridae viruses, or porcine Epidemic diarrhea virus (PEDV).

Preferably, the coronavirus is selected from one or more of MERS-CoV, SARS-CoV, and SARS-CoV-2, and the coronavirus is preferred as SARS-CoV-2.

The invention also provides an application of substance A in the preparation of drugs for the treatment and/or prevention of coronavirus-related diseases, in the form of A triazine derivative or a pharmaceutically acceptable salt thereof as indicated in formula (I′) or (III), or a composition as described above.

The invention also provides the application of a substance A′ in the preparation of drugs for the treatment and/or prevention of coronavirus-related diseases in the form of a triazine derivative or a pharmaceutically acceptable salt thereof as indicated in formula (I′), (I), (III) or (VI), or a composition as described above.

The coronavirus-associated disease can be MERS, SARS, or COVID-19, preferably COVID-19.

Unless otherwise stated, the following terms appearing in the specification and claims of the invention have the following meanings:

The term “pharmaceutically acceptable” means that salts, solvents, excipients, etc., are generally non-toxic, safe, and suitable for use by patients. The “patient” is preferably a mammal, and more preferably a human.

The term “pharmaceutically acceptable salt” means a salt prepared from the compound of the invention with a relatively non-toxic, pharmaceutically acceptable acid or base. When the compounds of the invention contain relatively acidic functional groups, alkali addition salts can be obtained by contacting the prototype of such compounds with a sufficient amount of pharmaceutically acceptable alkali in a suitable inert solvent. Pharmaceutically acceptable alkali addition salts include but are not limited to, Lithium salt, sodium salt, potassium salt, calcium salt, aluminum salt, magnesium salt, zinc salt, bismuth salt, ammonium salt, diethanolamine salt. When the compounds of the invention contain relatively basic functional groups, acid addition salts can be obtained by contacting the prototype of such compounds with enough pharmaceutically acceptable acid in a suitable inert solvent. The pharmaceutically acceptable acids include inorganic acids, which include but are not limited to: Hydrochloric acid, hydrobromic acid, hydroiodic acid, nitric acid, carbonic acid, phosphoric acid, phosphorous acid, sulfuric acid, etc., said pharmaceutically acceptable acids include organic acids, said organic acids include but are not limited to: Acetic acid, propionic acid, oxalic acid, isobutyric acid, maleic acid, benzoic acid, succinic acid, octoic acid, fumaric acid, lactic acid, mandelic acid, phthalic acid, benzenesulfonic acid, p-toluenesulfonic acid, citric acid, salicylic acid, tartaric acid, mesylate, isonicotinic acid, acid citric acid, oleic acid, tannic acid, pantothenic acid, hydrogen tartrate, ascorbic acid, gentian acid, fumaric acid, gluconic acid, Saccharic acid, formic acid, ethanesulfonic acid, dihydroxynaphthoic acid (i.e. 4,4″-methylene-bis(3-hydroxy-2-naphthoic acid)), amino acids (e.g., glutamic acid, arginine), etc. When the compound of the invention contains relatively acidic and relatively basic functional groups, it can be converted into an alkali addition salt or an acid addition salt. See Berge et al for details, “Pharmaceutical Salts”, Journal of Pharmaceutical Science 66:1-19 (1977), or, faced of Pharmaceutical Salts: Propertics, Selection, and Use (P. H einrich Stahl and Camille G. Wermuth, cd., Wiley-VCH, 2002).

An alkyl group means an alkyl group having a total of 1, 2, 3, 4, 5 or 6 carbon atoms as defined below. The total number of carbon atoms in the simplified symbol does not include the carbon that may be present in the substituent of the group.

The term “therapy” refers to therapeutic therapy. When it comes to specific conditions, treatment means: (1) alleviates one or more biological manifestations of a disease or condition, (2) interferes with (a) one or more points in the biological cascade that causes or causes the condition, or (b) one or more biological manifestations of the condition, (3) ameliorates one or more symptoms, effects or side effects associated with the condition, or one or more symptoms, effects, or side effects associated with the condition or its treatment, or (4) slowing down the development of the condition or one or more biological manifestations of the condition.

The term “therapeutic effective amount” means the amount of a compound that, when administered to a patient, is sufficient to effectively treat the disease or condition described herein. The “therapeutic effective amount” will vary according to the compound, the condition and its severity, and the age of the patient to be treated, and can be adjusted according to the needs of those skilled in the field.

On the basis of not violating the common sense in the field, the above optimal conditions can be arbitrarily combined to obtain the best examples of the invention.

The reagents and raw materials used in the invention are commercially available.

The positive improvement effect of the invention is that the compound of the invention can significantly prolong the half-life, reduce the demand for dosage, reduce side effects, and expand the range of treatment window on the basis of retaining the effectiveness of SARS-CoV-2. Therefore, the invention has a very good prospect for making drugs for treating diseases related to coronavirus infection.

### ILLUSTRATED DESCRIPTION

FIG. 1: Powder diffraction pattern of SHEN210 (coacetate of compound 1-2).

FIG. 2: Powder diffraction pattern of SHEN211 (fumaric acid eutectic of compound 1-2).

FIG. 3: Powder diffraction pattern of SHEN212 (ternary eutectic of I-2).

### CONCRETE IMPLEMENTATION MODE

The invention is further described by means of embodiments below, but does not therefore limit the invention to the scope of the embodiments. Experimental methods not specified in the following embodiments shall be selected in accordance with the conventional methods and conditions, or in accordance with the product specification.

### Embodiment 1 Synthesis of Deuterated Compound 1-1

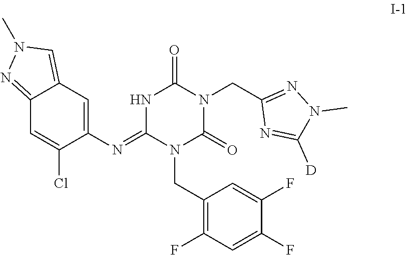

### Synthesis of Intermediate 1-03

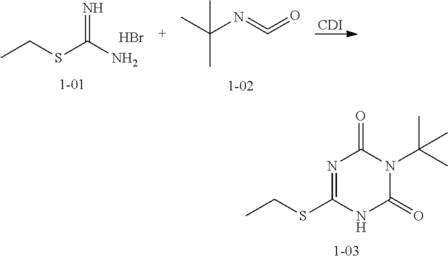

Tert-butyl isocyanate (1.2 mL, 10.5 mmol) and DBU (1.9 mL, 12.8 mmol) were added to the mixture of S-ethylthiurea hydrobromide (1.85 g, 10 mmol) and DMF (9.3 mL) under ice bath cooling and stirred for 6 hours. 1,1′-carbonyl diimidazole (1.95 g, 12 mmol) and DBU (1.9 mL, 12.8 mmol) were added to the reaction solution under an ice bath and stirred for 2 hours. Then 2 mol/L hydrochloric acid (80 mL) was slowly added, solid matter was precipitated, filtered, the solid was dissolved in ethyl acetate, dried with anhydrous magnesium sulfate, concentrated under reduced pressure, and purified by column chromatography. 1-03 was obtained with a yield of about 50%.

### Synthesis of Intermediate 1-6

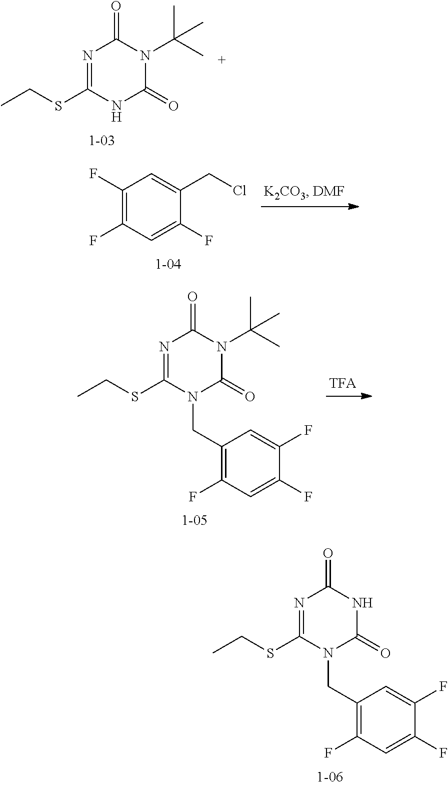 [SMILES: CCSc1nc(=O)n(C(C)(C)C)c(=O)[nH]1 ; Fc1cc(F)c(CCl)cc1F ; CCSc1nc(=O)n(C(C)(C)C)c(=O)n1Cc1cc(F)c(F)cc1F ; CCSc1nc(=O)[nH]c(=O)n1Cc1cc(F)c(F)cc1F]

Add potassium carbonate (17.97 g, 130 mmol) into acetonitrile (200 mL) of 1-03 (22.93 g, 100 mmol), 1-04 (22.60 g, 110 mmol) and stir for 3 hours under heating reflux. Filter the reaction liquid, concentrate and reduce the pressure to obtain the rent product of 3-tert-butyl-1-(2,4, 5-trifluorobenzyl)-6-(ethylthioyl)-1,3,5-triazine-2, 4-dione (1-05) as a slightly brown oil.

Trichloroacetic acid (100 mL) is added to the resulting rental product under cold temperature and stirred at room temperature for 17 hours. The reaction liquid is concentrated under reduced pressure to obtain 1-06 (2-step yield of about 85%), ¹H-NMR (400 MHz, CDCl3): δ 7.04-6.97 (m, 2H), 5.15 (s, 2H), 3.25-3.20 (q, 2H), 1.38-1.35 (t, 3H). LC-MS (ESI, m/z) 318 [M+H]⁺.

### Synthesis of Intermediate 1-08

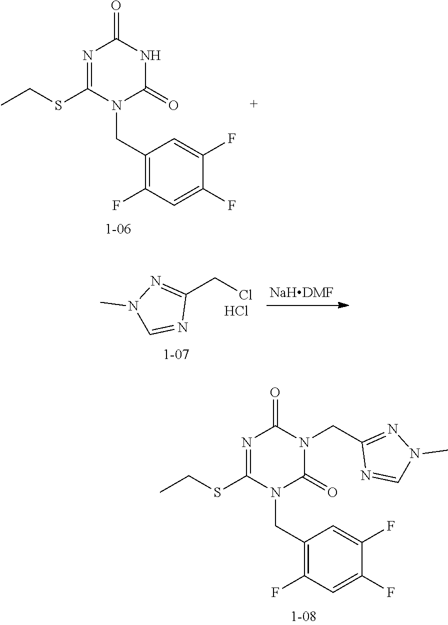

Dissolve 1-06 (2.50 g, 7.88 mmol) and 1-07 (1.99 g, 11.8 mmol) in DMF (23 mL) and add anhydrous potassium carbonate (3.27 g). 23.6 mmol), rising to 60° C., stirring reaction for 4 h, cooling to room temperature, the reaction liquid was poured into ammonium chloride aqueous solution, solid precipitation, filtration, water washing, solid recrystallization with ethanol to 1-08, the yield of about 50%, LC-MS (ESI): 413 [M+H]⁺.

### Synthesis of Intermediates 1-10

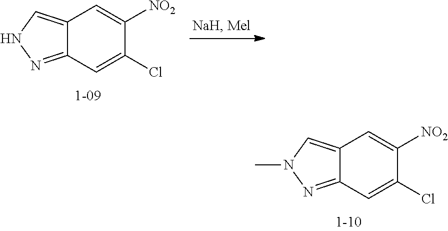

Add carbonic acid (562 mg, 4.07 mmol) to DMF (10 mL) solution of 5-nitro-6-chloro-1 h-indazole (334 mg, 1.35 mmol), stir at room temperature for 1 h, then slowly add iodomethanes (0.17 mL, 2.71 mmol), then stir for 2 h. The reaction mixture was diluted with methylene chloride, washed in saturated salt water, and dried with anhydrous sodium sulfate. Intermediates 1-10A and 1-10B purified by silica gel column chromatography, LC-MS (ESI): 214 [M+H]⁺,

### Synthesis of Intermediates 1-11

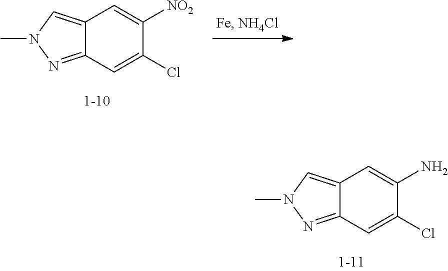

Dissolve 1-10 (0.39 g, 1.5 mmol) in ethanol (15 mL), add iron powder (0.42 g, 7.5 mL) and 0.4 mL hydrochloric acid, rise temperature for reflux reaction for 1 h, cool to room temperature, dilute the reaction solution with ethanol, filter with diatomaceous earth, concentrate, dissolve the residue with ethyl acetate, wash with sodium bicarbonate solution, water and salt water. Anhydrous sodium sulfate drying. Concentrated under vacuum and purified by silica gel column chromatography (PE/EA=4/1), 1-11, LC-MS (ESD): 182 [M+H]⁺.

### Synthesis of Intermediate 1-12 (i.e., Control Compound S-217622)

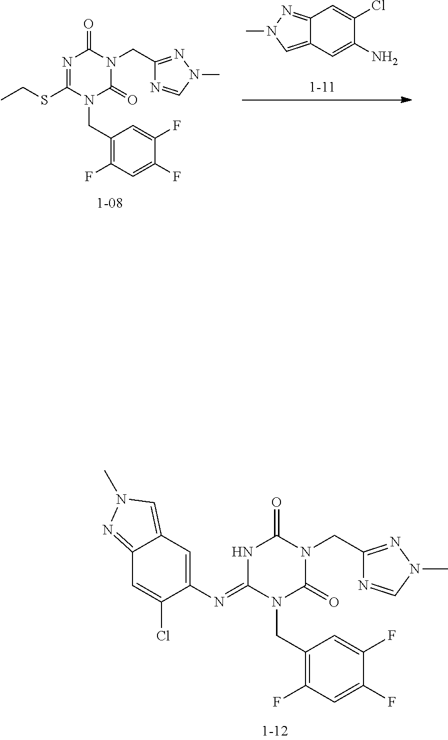

Dissolve 1-08 (5.0 g, 12.6 mmol), 1-11 (2.06 g, 18.9 mmol) in the mixture of acetic acid (11.32 g, 189 mmol) and tert-butanol (100 mL), stir at reflux at high temperature for 3 hours. cool to room temperature, and add the reaction solution to saturated sodium bicarbonate aqueous solution (500 mL). It was extracted with ethyl acetate (500 mL), combined with organic phase, washed, dried with anhydrous sodium sulfate, filtered, concentrated under reduced pressure, and the residue was separated by silica gel column chromatography (gradient elelation with dichloromethane and methanol, 0-20% MeOH) to obtain 1-12, ¹HNMR (400 MHz, DMSO-80DCI=4:1) δ9.05 (s, 1H), 8.41 (s, 1H), 7.80 (s, 1H), 7.57 (s, 1H), 7.46-7.55 (m, 2H), 5.29 (s, 2H), 5.08 (s, 2H), 3.92 (s, 3H); LC-MS (ESI): 532 [M+H]⁺.

### Synthesis of Deuterated Compound 1-1

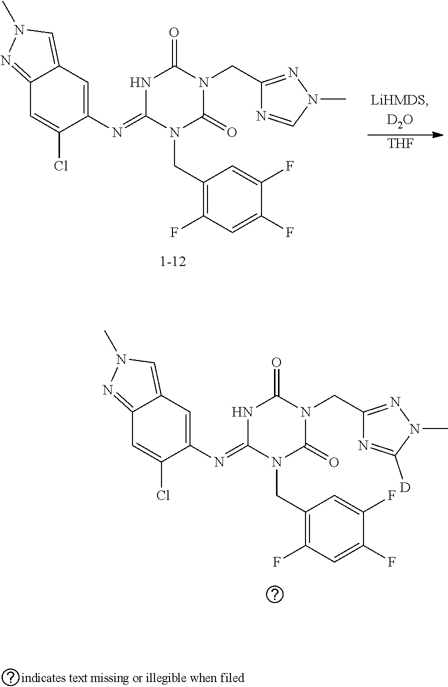

Dissolve 1-12 in anhydrous tetrahydrofuran, cool it in ice bath, add LiHMDS in drops, then add heavy water in drops after 30 min, stir for 1 h, pour the reaction liquid into water, adjust pH to about 6-7 with hydrochloric acid, extract with ethyl acetate, combine organic phase, dry and concentrate with anhydrous sodium sulfate, and purify the compound by silica gel column chromatography. LC-MS (ESI): 533 [M+H]⁺.

### Embodiment 2 Synthesis of Deuterated Compounds 1-2

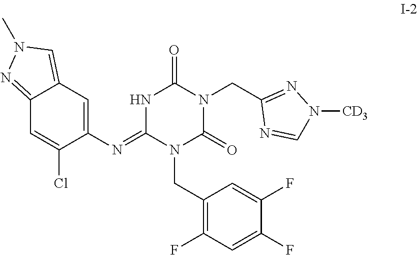 [SMILES: [2H]C([2H])([2H])n1cnc(Cn2c(=O)[nH]/c(=N\c3cc4cn(C)nc4cc3Cl)n(Cc3cc(F)c(F)cc3F)c2=O)n1]

### Synthesis Method 1 of Deuterated Compound 1-2

### Synthesis of Intermediate 2-04

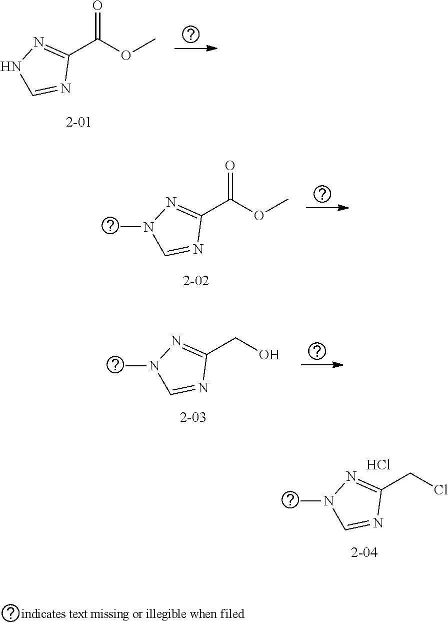

Dissolve 2-01 in anhydrous tetrahydrofuran, cool it in ice bath, add LiHMDS, react for about 30 min, then add deuteriodomethane in drops, then when it rise to room temperature, stir it to react for 2 h, then quenching with water, extract dichloromethane, combine organic phase, wash in saturated salt water, dry with anhydrous sodium sulfate, filter and concentrate, and purify 2-02 by column chromatography. ¹HNMR (400 MHz, CDCl3) δ 8.17 (s, 1H), 4.01 (s, 3H).

Will 2-01 (127.1 g. 1.0 mol) dissolved in anhydrous tetrahydrofuran (1000 mL), cooled in ice bath, added sodium hydride (60%, 44 g), reacted for about 30 min, then added deuteriodomethane (159.5 g, 1.1 mol) in drops, then raised to room temperature for stirring reaction for 2 h, quenched with water, extracted dichloromethane, combined with organic phase, and dissolved in water. Wash in saturated salt water and dry with anhydrous sodium sulfate. 2-02 (65.3 g, yield 45%), ¹HNMR (400 MHz, CDCl₃) δ 8.17 (s, 111), 4.01 (s, 3H) were obtained by filtration and column chromatography. LC-MS (ESI) 145.1 [M+H]⁺.

Under the protection of nitrogen, 2-02 was dissolved in anhydrous tetrahydrofuran, and the tetrahydrofuran solution of lithium aluminum tetrahydro was added in drops, the reflux reaction was heated for 1 h, and then cooled to room temperature, 1 ml of water was slowly added for quenching, and then anhydrous magnesium sulfate was added, stirred for 1 h, filtered and concentrated. 2-03, ¹H NMR (400 MHz, CDCl3) δ 8.04 (d, J=36.0 Hz, 1H), 4.76 (s, 2H), 3.48 (s, 1H) were obtained by column chromatography.

Under nitrogen protection, 2-02 (57.6 g, 400 mmol) was dissolved in anhydrous tetrahydrofuran (500 mL), and lithium aluminum tetrahydro (18.5 g) was added in drops. 440 mmol) tetrahydrofurmine (100 mL) solution was heated, the reflux reaction 1 h was heated, cooled to room temperature, slowly added to water quenching, then added anhydrous magnesium sulfate, stirred for 1 h, filtered, concentrated, and purified by column chromatography to obtain 2-03 (37.9 g, yield 81%), ¹HNMR (400 MHz, CDCl3) δ 8.04 (d, J=36.0 Hz, 1H), 4.76 (s, 2H), 3.48 (s, 1H); LC-MS (ESI) 117.1 [M+1]⁺.

Dissolve 2-03 in anhydrous dichloromethane, add sulfoxide chloride, stir at room temperature for 3 h, concentrate under reduced pressure to remove solvent and excess to obtain sulfoxide chloride, residue with ether beating, filtration, vacuum drying, 2-04.LC-MS (ESI): 135 [M−H]⁺.

Dissolve 2-03 (35.1 g, 300 mmol) in anhydrous dichloromethane (100 mL), add sulfoxide chloride (50 mL), stir at room temperature for 3 h, concentrate under pressure to remove solvent and excess to sulfoxide chloride, and the residue is beaten with ether, filtered, vacuum dried to obtain 2-04 (46.5 g, 90% yield). ¹HNMR (400 MHz, DMSO) δ 8.06 (s, 1H), 4.78 (s, 2H); LC-MS (ESI): 135 [M+H]⁺.

### Synthesis of Intermediate 2-05

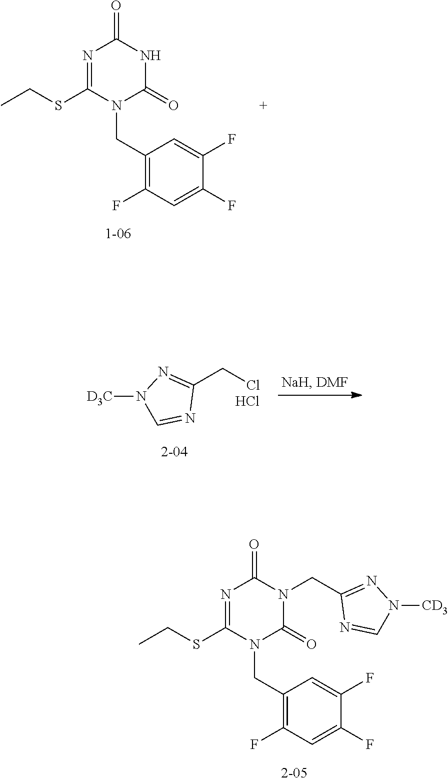 [SMILES: CCSc1nc(=O)[nH]c(=O)n1Cc1cc(F)c(F)cc1F ; [2H]C([2H])([2H])n1cnc(CCl)n1 ; Cl ; [2H]C([2H])([2H])n1cnc(Cn2c(=O)nc(SCC)n(Cc3cc(F)c(F)cc3F)c2=O)n1]

Dissolve 1-06 (2.50 g, 7.88 mmol) and 2-04 (1.99 g, 11.8 mmol) in DMF (23 mL) and add anhydrous potassium carbonate (3.27 g, 23.6 mmol), temperature rise to 60° C., stirring reaction for 4 h, cooling to room temperature, the reaction liquid is poured into ammonium chloride aqueous solution, solid precipitation, filtration, water washing, solid recrystallization with ethanol to 2-05 (2.32 g, yield 71%), ¹HNMR (400 MHz, DMSO-d6), δ 8.36 (s, 1H), 7.57-7.70 (m, 1H), 7.38-7.52 (m, 1H), 5.12 (s, 2H), 4.98 (s, 2H), 3.15 (q, 2H), 1.28 (L, 3H); LC-MS (ESI): 416 [M+H]⁺.

### Synthesis of Target Compounds 1-2

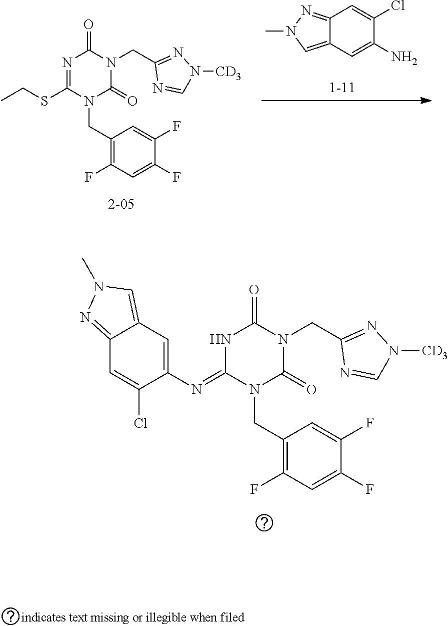 [SMILES: [2H]C([2H])([2H])n1cnc(Cn2c(=O)nc(SCC)n(Cc3cc(F)c(F)cc3F)c2=O)n1 ; Cn1cc2cc(N)c(Cl)cc2n1 ; [2H]C([2H])([2H])n1cnc(Cn2c(=O)[nH]/c(=N\c3cc4cn(C)nc4cc3Cl)n(Cc3cc(F)c(F)cc3F)c2=O)n1]

Dissolve 2-05 (5.0 g, 12.6 mmol), 1-11 (2.06 g, 18.9 mmol) in the mixture of acetic acid (11.32 g, 189 mmol) and tert-butanol (100 mL), stir at reflux at high temperature for 3 hours, cool to room temperature, and add the reaction solution to saturated sodium bicarbonate aqueous solution (500 mL). It was extracted with ethyl acetate (500 mL), combined with organic phase, washed, dried with anhydrous sodium sulfate, filtered, concentrated under reduced pressure, and the residue was separated by silica gel column chromatography (gradient elelation with dichloromethane and methanol) to obtain 1-2 (3.22 g, 50% yield), ¹HNMR (400 MHZ, DMSO-d6): 4.14 (s, 3H), 4.92 (s, 2H), 5.23 (s, 2H), 7.45-7.65 (m, 2H), 7.71 (s, 1H), 8.29 (s, 1H), 8.35 (s, 1H); ¹HNMR (400 MHz, Pyridine-d5) δ 8.33 (s, 1H), 8.02-7.91 (m, 1H), 7.87 (s, 2H), 7.86 (s, 1H), 7.38 (s, 1H), 7.13-7.22 (m, 1H), 5.66 (s, 3H), 5.57 (s, 3H), 3.99 (s, 3H); LC-MS (ESI, m/z) 535 [M+H]⁺.

### Synthesis Method 2 of Deuterated Compound 1-2

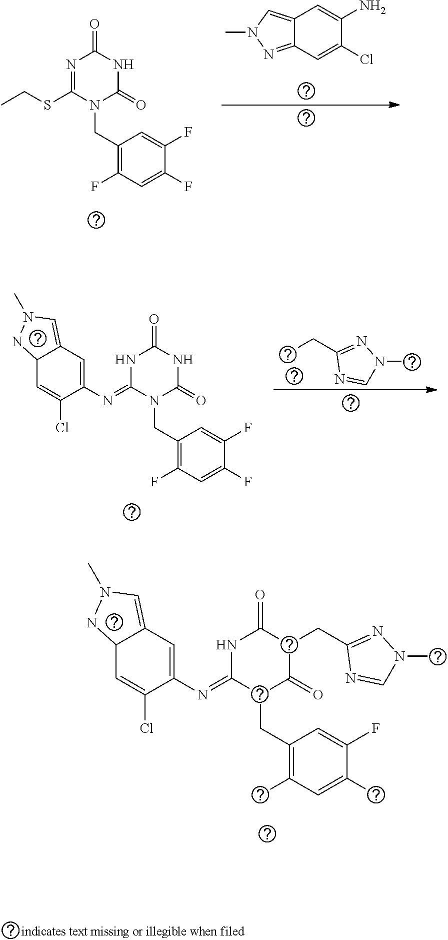

### Synthesis of Compound 2-06

Make 1-06 (6.35 g. 20.0 mmol) and 1-11 (5.45 g, 30.0 mmol) dissolved in acetic acid (50 mL), stir and reflux at high temperature for 5 hours, cool to room temperature, solid precipitates, filtration, washing, drying, 2-06 (4.63 g, 53% yield). ¹H-NMR (400 MHz, DMSO-d6): δ 11.33 (s, 1H), 11.63 (s, 1H), 8.26 (s, 1H), 7.69-7.56 (m, 3H), 7.13 (s, 1H), 5.15 (s, 2H), 4.16 (s, 3H); MS (ESI, m/z) 437.2 [m+H]⁺.

### Synthesis of Compounds 1-2

Put 2-06 (4.19 g, 10.0 mmol) and 2-04 (2.56 g, 15.0 mmol) dissolved in DMA (23 mL), then anhydrous cesium carbonate (4.14 g, 30.0 mmol) is added, and the temperature is raised to 60° C. for stirring reaction for 6 h. Cool to room temperature, the reaction liquid is poured into ammonium chloride aqueous solution, solid precipitation, filtration, water washing, drying to obtain crude products, purified by silica gel column chromatography (gradient elution of dioxomethane/methanol), 1-2 (2.25 g yield 35%). ¹H NMR (400 MHZ, DMSO-d6): 4.14 (s, 3H), 4.92 (s, 2H), 5.23 (s, 2H), 7.45-7.65 (m, 2H), 7.71 (s, 1H), 8.29 (s, 1H), 8.35 (s, 1H); ¹H NMR (400 MHz, Pyridine-d5) δ 8.33 (s, 1H), 8.02-7.91 (m, 1H), 7.87 (s, 2H), 7.86 (s, 1H), 7.38 (s, 1H), 7.13-7.22 (m, 1H), 5.66 (s, 3H), 5.57 (s, 3H), 3.99 (s, 3H); LC-MS (ESI, m/z) 535 [M+H]⁺.

### Synthesis of Compounds 1-3 in Embodiment 3

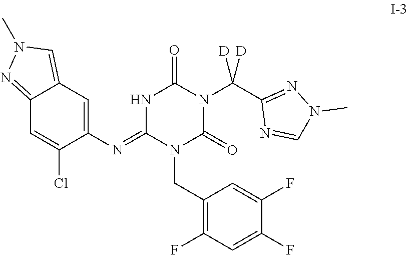

### Synthesis Method 1 of Deuterated Compound 1-3

### Synthesis of Intermediate 3-03

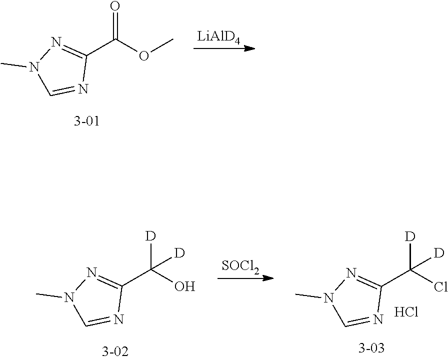

Under the protection of nitrogen, put tetratritium aluminum lithium suspended in anhydrous tetrahydrofuran, drip into the solution of tetrahydrofuran dissolved in 1-methyl-1 h-1,2, 4-triazole-3-methyl formate, raise the temperature for reflux for 1 h, cool, quench with 1 mL, then add anhydrous magnesium sulfate, stir for 30 min, filter, concentrate to dry, and 3-02 is obtained. ¹HNMR (400 MHz, DMSO-d6) δ 8.23 (s, 1H), 3.81 (s, 3H), 3.20 (s, 1H).

Under the protection of nitrogen, put tetratritium aluminum-lithium (5.1 g, 110 mmol) suspended in anhydrous tetrahydrofuran (50 mL), and make the solution of tetrahydrofuran (200 mL) in 1-methyl-1 h-1,2, 4-triazole-3-methyl formate (14.1 g, 100 mmol) added in drops, and then heat it and reflux for 1 h. Cool and add 5 mL water to quench, then add anhydrous magnesium sulfate, stir for 30 min, filter, concentrate until dry, 3-02 (8.1 g, 70% yield) is obtained. ¹H NMR (400 MHz, DMSO-d6) δ 8.23 (s, IH), 3.81 (s, 3H), 3.20 (s, 1H): LC-MS (ESI): 116.1 [M+H]⁺.

Redissolve the 3-02 obtained above with anhydrous dichloromethane, add with sulphoxide chloride, stir at room temperature for 3 h, the reaction is detected by TLC, concentrate under reduced pressure, and the residue is beaten with ether, filter, and dry in vacuum to obtain 3-03, ¹HNMR (400 MHz, DMSO-d6) δ 8.43 (s, 1H), 3.82 (s, 3H); LC-MS (ESI): 134 [M+H]⁺.

Put the 3-02 obtained above redissolved with anhydrous dichloromethane (100 mL), and add the sulfone chloride (10 mL), stir at room temperature for 3 h. After the TLC test is completed, the reaction is concentrated under reduced pressure, and the residue is beater with ether, filter, and dry in vacuum to obtain 3-03 (9.38 g, 90% yield). ¹H NMR (400 MHz, DMSO-d6) δ 8.43 (s, 1H), 3.82 (s, 3H); LC-MS (ESD): 134 [M+H]⁺.

### Synthesis of Intermediate 3-04

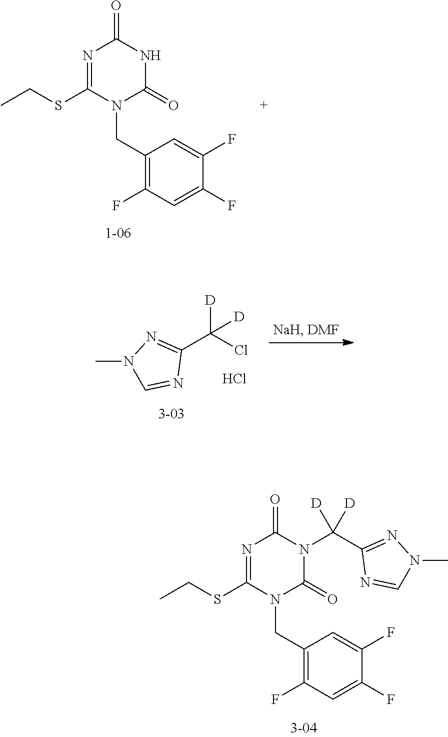

Let 1-06 (2.50 g, 7.88 mmol) and 3-03 (1.99 g, 11.8 mmol) dissolved in DMF (23 mL), adding anhydrous potassium carbonate (3.27 g, 23.6 mmol), stirring at 60° C. for 4 h, cooling to room temperature, pouring the reaction liquid into ammonium chloride aqueous solution, solid precipitation, filtration, water washing, solid recrystallization with ethanol to produce intermediates 3-04 (2.28 g, 70%). ¹H NMR (400 MHz, DMSO d6) δ 8.36 (s, 1H), 7.58-7.69 (m, 1H), 7.51-7.37 (m, 1H), 5.12 (s, 2H), 3.80 (s, 3H), 3.14 (q, 2H), 1.28 (t, 3H); LC-MS (ESI): 415 [M+H]⁺.

### Synthesis of Deuterated Compounds 1-3

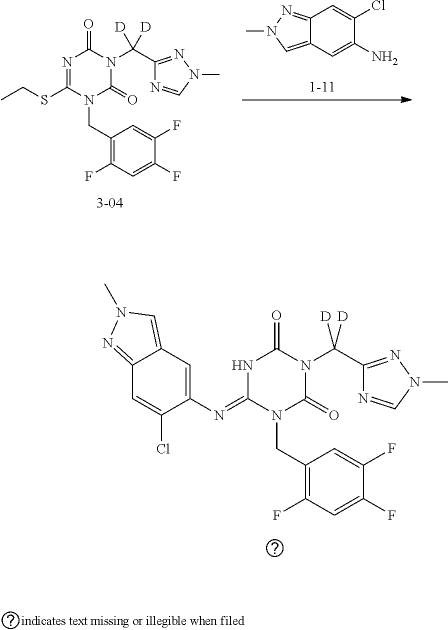

Dissolve 3-04 (5.0 g, 12.6 mmol) and 1-11 (2.06 g, 18.9 mmol) in the mixture of acetic acid (11.32 g, 189 mmol) and tert-butanol (100 mL), heating, refluxing, stirring and reacting for 3 hours, cool to room temperature, and add the reaction solution to saturated sodium bicarbonate aqueous solution (500 mL). It was extracted with ethyl acetate (500 mL), combined with organic phase, washed, dried with anhydrous sodium sulfate, filtered, concentrated under reduced pressure, and the residue was separated by silica gel column chromatography (gradient elelation with dichloromethane and methanol, 0-20% MeOH) to obtain 1-3 (3.34 g), 50% yield), ¹H NMR (400 MHz, DMSO-d6) δ 11.05 (s, 0.6H, NH), 9.70 (s, 0.4H, NH), 8.36 (s, 1H), 8.28 (s, 1H), 7.71 (s, 1H), 7.40-7.65 (m, 2H), 7.15 (s, 1H), 5.23 (s, 2H), 4.15 (s, 3H), 3.80 (s, 3H); LC-MS (ESI): 534 [M+H]⁺.

### Synthesis Method 2 of Deuterated Compound

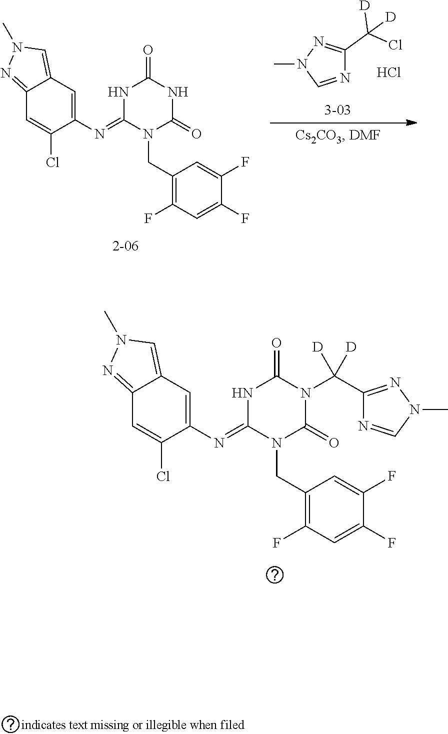

Dissolve 2-06 (4.19 g. 10.0 mmol) and 3-03 (2.56 g, 15.0 mmol) in DMA (23 mL) and add anhydrous cesium carbonate (4.14 g). Then raise the temperature to 50° C. and stir it for reaction for 3 hours. Cool it to room temperature. Pour reaction liquid into ammonium chloride solution, solid precipitated, filtered, washed in water, dried to coarse product, purified by silica gel column chromatography (gradient elution of dichloromethane/methanol), get I-3 (2.25 g yield 35%). ¹H NMR (400 MHz, DMSO-d6) δ 11.05 (s, 0.6H, NH), 9.70 (s, 0.4H, NH), 8.36 (s, 1H), 8.28 (s, 1H), 7.71 (s, 1H), 7.40-7.65 (m, 2H), 7.15 (s, 1H), 5.23 (s, 2H), 4.15 (s, 3H), 3.80 (s, 3H); LC-MS (ESI, m/z) 534 [M+H]⁺.

### Embodiment 4 Synthesis of Deuterated Compounds 1-4

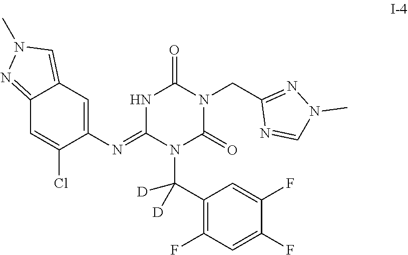

### Synthesis of Intermediate 4-03

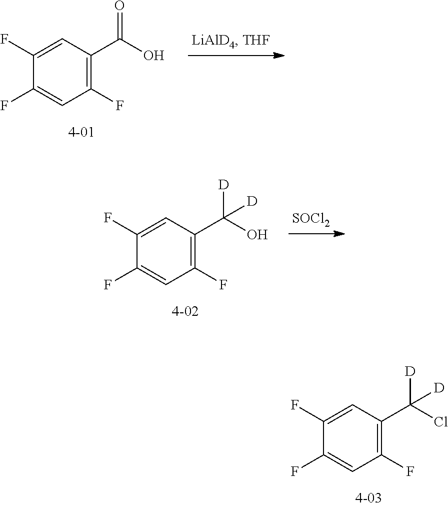

Under the protection of nitrogen, Suspend and dissolve tetradeuterium aluminum lithium in anhydrous tetrahydrofuran, drip into the solution of tetrahydrofuran dissolved in 2,4,5-trifluorobenzoic acid, then raise temperature and reflux for 1 h, cooling, quench with 1 mL, then add anhydrous magnesium sulfate, stirring for 30 min, filtered, concentrated to dry, 4-02 was obtained, which was directly used in the next step.

Under nitrogen protection, Suspend and dissolve tetratritium aluminum lithium (10.2 g, 220 mmol) in anhydrous tetrahydrofuran (100 mL), and add solution of tetrahydrofuran (200 mL) dissolved in 2,4,5-trifluorobenzoic acid (35.2 g, 200 mmol), then raise temperature and reflux for 1 h, cooling, quench with 5 mL water, then anhydrous magnesium sulfate was added, stirred for 30 min, filtered, concentrated to dry, 4-02 (29.8 g, 90% yield) was obtained.

Dissolve 4-02 with anhydrous dichloromethane, add sulphoxide chloride, stir at room temperature for 3 h, TLC test is completed, pour the reaction liquid into ice water, extract with dichloromethane 3 times, combine organic phase, wash with saturated salt water, sodium bicarbonate and water, anhydrous sodium sulfate dry, filter, concentrate, obtain 4-03, LC-MS (ESI): 183 [M+H]⁺.

Redissolve 4-02 (29.8 g) with anhydrous dichloromethane, add sulphoxide chloride (20 mL), stir at room temperature for 3 h, TLC test is completed, pour the reaction liquid into ice water, extract with dichloromethane 3 times, combine organic phase, wash with saturated salt water, sodium bicarbonate and water, anhydrous sodium sulfate dry, filter and concentrate, 4-03 (32.4 g, yield of 98%), ¹H NMR (300 MHz, CDCl₃) δ 7.34-7.24 (m, 1H), 6.69-6.88 (m, 1H); LC-MS (ESI): 183 [M+H]⁺.

### Synthesis of Intermediate 4-05

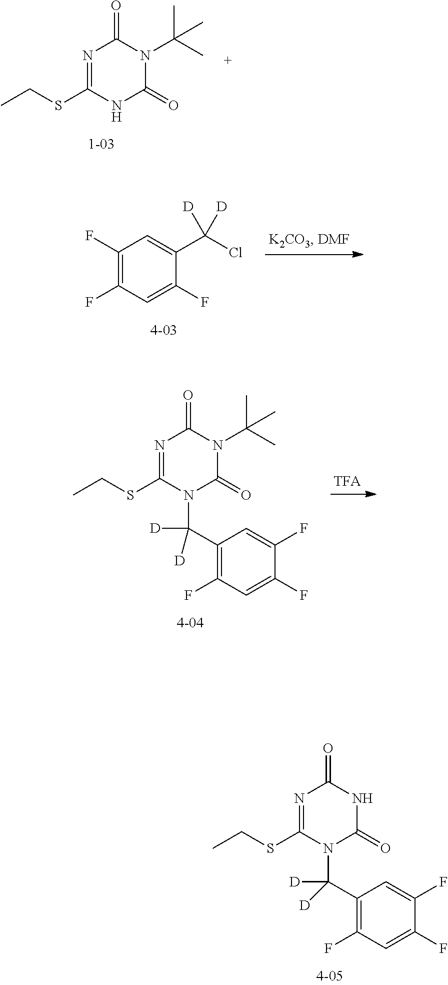 [SMILES: CCSc1nc(=O)n(C(C)(C)C)c(=O)[nH]1 ; [2H]C([2H])(Cl)c1cc(F)c(F)cc1F ; [2H]C([2H])(c1cc(F)c(F)cc1F)n1c(SCC)nc(=O)n(C(C)(C)C)c1=O ; [2H]C([2H])(c1cc(F)c(F)cc1F)n1c(SCC)nc(=O)[nH]c1=O]

Add potassium carbonate (17.97 g, 130 mmol) into acetonitrile solution (200 mL) of 1-03 (22.93 g, 100 mmol) and 4-03 (22.60 g, 110 mmol). Stir for 3 hours while heating and refluxing. Filter the reaction liquid, concentrate the filtrate under reduced pressure, and obtain 4-04 (33.8 g, 90%), which is directly used for the next reaction. ¹H NMR (400 MHz, CDCl₃) δ 7.04-6.92 (m, 2H), 3.18 (q, 2H), 1.67 (s, 9H), 1.36 (t, 3H)

Dissolve 4-05 obtained in the previous step in dichloromethane under ice bath cooling, and add trifluoroacetic acid and stir it at room temperature for 24 hours. The reaction liquid is concentrated under reduced pressure to obtain 4-05, LC-MS (ESI): 320 [M+H]⁺.

Dissolve 4-05 (30.0 g, 0.69 mmol) obtained in the previous step in dioxomethane (100 mL) under ice bath cooling, and trifluoroacetic acid (100 mL) is added and stirred at room temperature for 24 hours. The reaction solution is reduced pressure and concentrated to obtain 4-05 (25.5 g, 99%), 1H NMR (400 MHZ, CDCl₃) δ (s, 1H). 8.81 7.20-6.93 (m, 2H), 3.28 (q. 2H), 1.42 (t, 3H); LC-MS (ESI): 320 [M−H]⁺.

### Synthesis of Intermediate 4-06

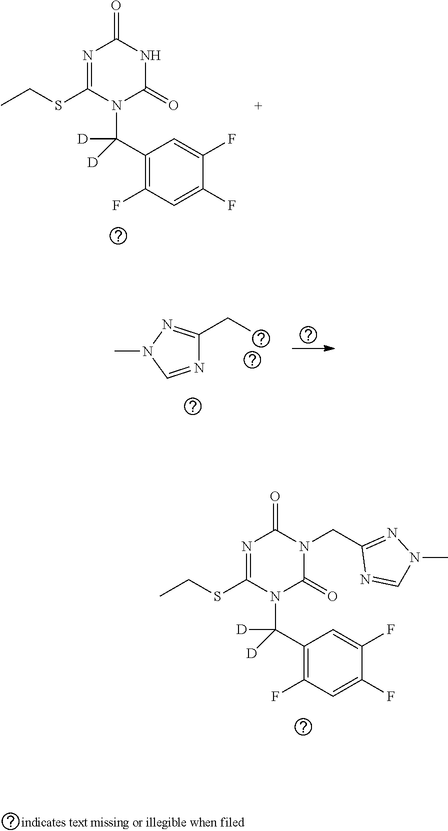

Dissolve 4-05 (2.50 g, 7.88 mmol) and 1-07 (1.99 g, 11.8 mmol) in DMF (23 mL) and add anhydrous potassium carbonate (3.27 g, 23.6 mmol), raise temperature to 60° C. and stir for reaction for 4 h. Cool to room temperature and pour the reaction liquid into ammonium chloride solution, solid precipitation, filtration, water washing, solid recrystallization with ethanol to 1-08 (2.36 g, yield 72%), ¹H NMR (400 MHz, CDCl₃) δ 8.00 (d, 1H), 7.26-7.13 (m, 1H), 7.05-6.96 (m, 1H), 5.30 (s, 2H), 3.92 (d, 3H), 3.27 (q, 2H), 1.40 (t, 3H); LC-MS (ESI): 415.1 [M+H]⁺.

### Synthesis of Deuterated Compounds 1-4

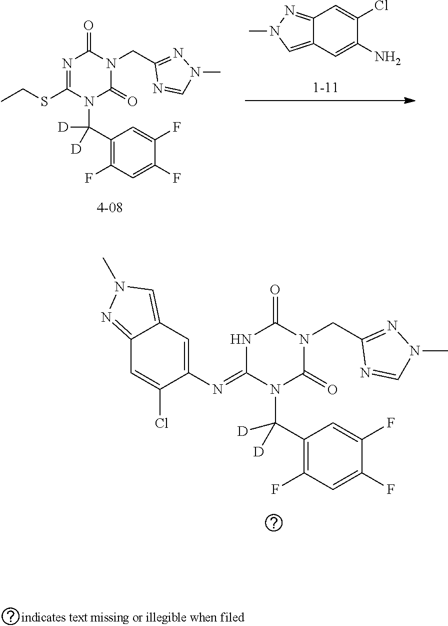

Dissolve 4-06 (5.0 g, 12.6 mmol) and 1-11 (2.06 g, 18.9 mmol) in a mixture of acetic acid (11.32 g, 189 mmol) and tert-butanol (100 mL), raise the temperature, reflux and stir it for reaction for 3 hours, cool to room temperature. Add the reaction solution into saturated sodium bicarbonate aqueous solution (500 mL), extract with ethyl acetate (500 mL), combine with organic phase, washed, dried with anhydrous sodium sulfate, filtered, concentrated under reduced pressure, and make the residue separated by silica gel column chromatography (gradient elelation with dichloromethane and methanol, 0-20% MeOH) to obtain compounds 1-4 (3.09 g, 48%). ¹H NMR (400 MHz, DMSO) δ 11.07 (s, 0.65H), 9.68 (s, 0.35H), 8.23-8.44 (m, 2H), 7.59-7.81 (m, 2H), 7.13 (s, 1H), 4.92 (s, 2H), 4.14 (s, 3H), 3.81 (s, 3H); LC-MS (ESD): 534.1 [M+H]⁺.

### Embodiment 5 Synthesis of Deuterated Compounds 1-5

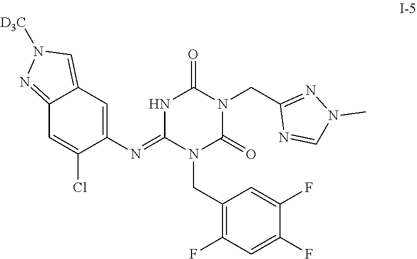 [SMILES: [2H]C([2H])([2H])n1cc2cc(/N=c3\[nH]c(=O)n(Cc4ncn(C)n4)c(=O)n3Cc3cc(F)c(F)cc3F)c(Cl)cc2n1]

### Synthesis of Intermediate 5-01

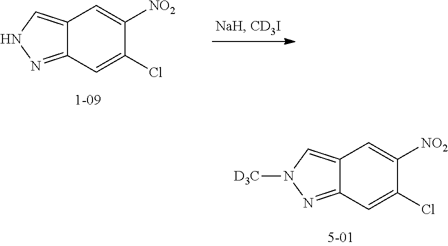 [SMILES: O=[N+]([O-])c1cc2c[nH]nc2cc1Cl ; [2H]C([2H])([2H])n1cc2cc([N+](=O)[O-])c(Cl)cc2n1]

Dissolve 1-09 (334 mg, 1.35 mmol) in DMF (10 mL), cool it in ice bath, add sodium hydride (562 mg, 4.07 mmol), stir for 30 min, then slowly add deiodomethanes (0.17 mL, 2.71 mmol), increase the temperature to room temperature and continue to stir for 2 h. The reaction mixture is diluted with dichloromethane, washed in saturated salt water, dried with anhydrous sodium sulfate, filtered and concentrated, and the residue is purified by silica gel column chromatography to obtain 5-01.

¹HNMR (400 MHz, DMSO-d6) δ 8.74 (s, 1H), 8.69 (s, 1H), 8.01 (s, 1H); LC-MS (ESI): 215 [M+H]⁺.

### Synthesis of Intermediate 5-02

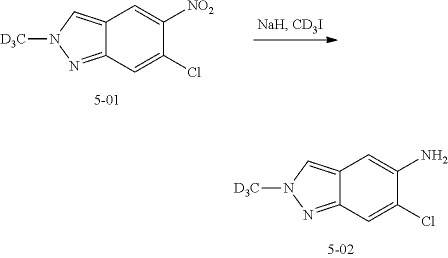 [SMILES: [2H]C([2H])([2H])n1cc2cc([N+](=O)[O-])c(Cl)cc2n1 ; [2H]C([2H])([2H])n1cc2cc(N)c(Cl)cc2n1]

Dissolve 5-01 (0.39 g, 1.5 mmol) in ethanol (15 mL), add iron powder (0.42 g, 7.5 mL) and 0.4 mL hydrochloric acid, rise temperature for reflux reaction for 1 h, cool to room temperature, dilute the reaction solution with ethanol, filter with diatomaceous earth, concentrate, dissolve the residue with ethyl acetate, wash with sodium bicarbonate solution, water and salt water. Anhydrous sodium sulfate drying. Concentrated under reduced pressure and purified by silica gel column chromatography (PE/EA=4/1), 5-02 (0.24 g, 87%) is obtained. ¹H NMR (400 MHz, DMSO-d6) δ 7.98 (s, 1H), 7.58 (s, 1H), 6.87 (s, 1H), 4.95 (s, 2H); LC-MS (ESI): 185 [M+H]⁺.

### Synthesis of Compounds 1-5

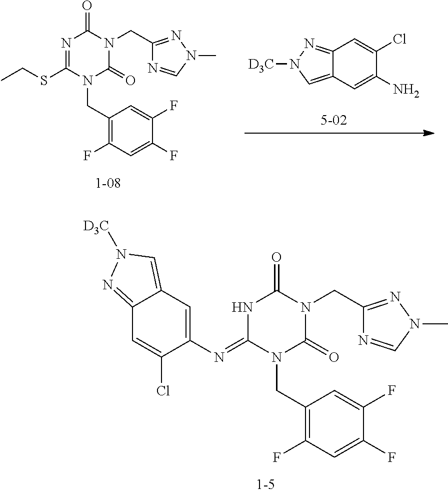 [SMILES: CCSc1nc(=O)n(Cc2ncn(C)n2)c(=O)n1Cc1cc(F)c(F)cc1F ; [2H]C([2H])([2H])n1cc2cc(N)c(Cl)cc2n1 ; [2H]C([2H])([2H])n1cc2cc(/N=c3\[nH]c(=O)n(Cc4ncn(C)n4)c(=O)n3Cc3cc(F)c(F)cc3F)c(Cl)cc2n1]

Dissolve 1-08 (5.0 g, 12.6 mmol) and 5-02 (2.06 g, 18.9 mmol) in a mixture of acetic acid (11.32 g, 189 mmol) and tert-butanol (100 mL), raise the temperature, reflux and stir it for reaction for 3 hours, cool to room temperature. Add the reaction solution into saturated sodium bi carbonate aqueous solution (500 mL), extract with ethyl acetate (500 mL), combined with organic phase, washed with water, dried with anhydrous sodium sulfate, filtered, concentrated un der reduced pressure. The residue is separated by silica gel column chromatography (gradient elution with dichloromethane-methanol, 0-20% MeOH) to obtain compounds 1-5, ¹HNMR (400 MHz, DMSO-d₆) δ (11.07s, 0.6H, NH), 9.68 (s, 0.4H, NH), 8.36 (s, 1H), 8.23 (s, 1H), 7.35-7.85 (m, 3H), 7.13 (s, 1H), 5.20 (s, 2H), 4.92 (s, 2H), 3.80 (s, 3H); LC-MS (ESI): 535 [M+H]⁺.

### Embodiment 6 Synthesis of Deuterated Compounds 1-6

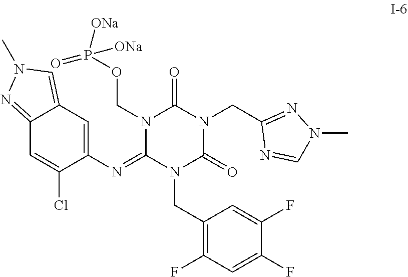

### Synthesis of Intermediate 6-01

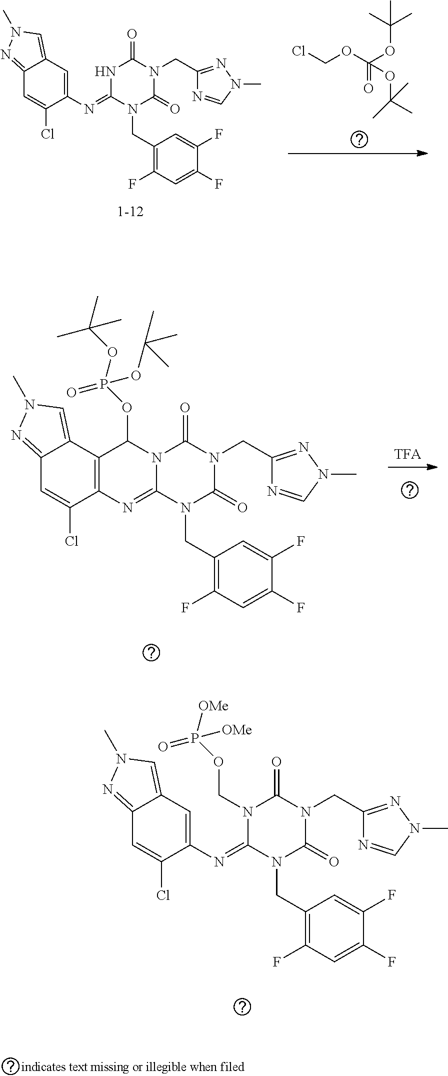

Dissolve compounds 1-12 in DMF, cool it in ice bath, add with sodium hydride, stir for about 30 min, then slowly added with di-tert-butyl chloromethyl phosphate, increase to room temperature and stir for reaction for 3 h. Compound 6-02, LC-MS (ESI): 754 [M+H]⁺ is obtained after the reaction solution is diluted with ethyl acetate, washed in saline solution, dried with anhydrous sodium sulfate, concentrated and purified by silica gel column chromatography.

Dissolve 6-02 in dichloromethane, add trifluoroacetic acid, stir for reaction 1 h, reduce pressure and concentrate to remove solvent and excess trifluoroacetic acid, the residue is re-dissolved with ethanol, add sodium hydroxide ethanol solution, stir 1 h, solid precipitation, filtration, dried, then obtain compounds 1-6.

### Examples 7-11

Using the same synthesis method of Embodiment 6, let 1-1, 1-2, 1-3, 1-4, 1-5, etc., reacted with 6-01 respectively, and then the tert-butyl group is removed by trifluoroacetic acid to obtain compounds 1-7, 1-8, 1-9, 1-10, and 1-11, respectively.

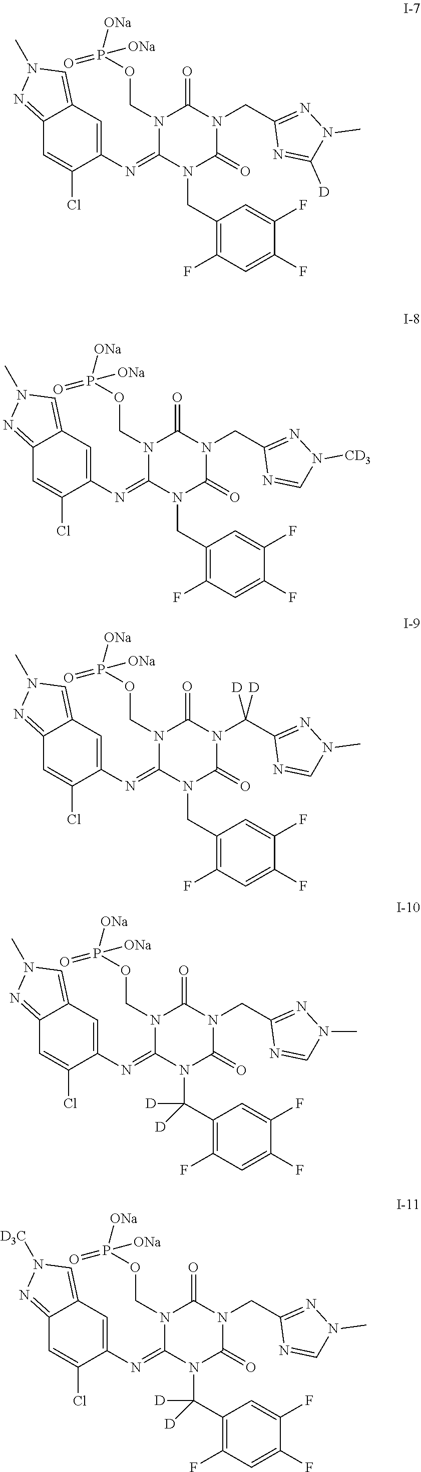

### Embodiment 12 Synthesis of Compounds 1-12

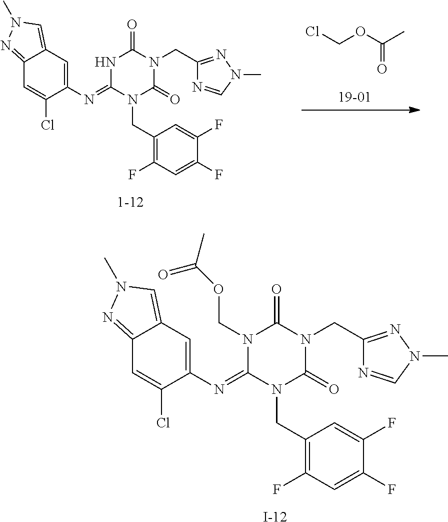

Dissolve compounds 1-12 in DMF, cool in ice bath, add sodium hydride, stir for about 30 min, and then slowly add chloromethyl acetate, heat to room temperature stirring reaction for 3 h. The reaction solution is diluted with ethyl acetate, washed in salt water, dried with anhydrous sodium sulfate, concentrated, and purified by silica gel column chromatography to obtain compound I-12, LC-MS (ESI): 604 [M+H]⁺.

### Embodiment 13 Synthesis of Compounds 1-13

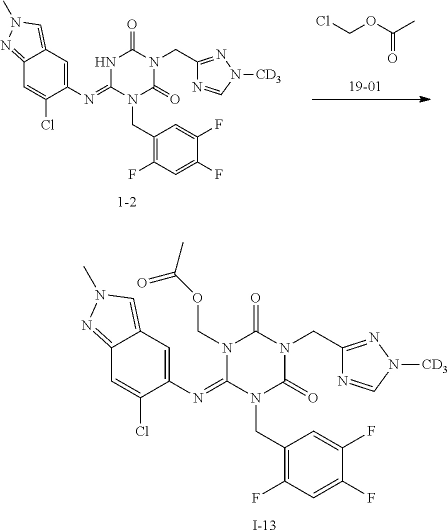

Dissolve compounds 1-2 in DMF, cool in ice bath, add sodium hydride, stir for about 30 min, then slowly add chloromethyl acetate, increase to room temperature and stir for reaction for 3 h. The reaction solution is diluted with ethyl acetate, washed in salt water, dried and concentrated with anhydrous sodium sulfate, and purified by silica gel column chromatography and compounds 1-13 is obtained, H NMR (400 MHz, DMSO-d₆) 1.23 (s, 3H), 4.12 (s, 3H), 4.97 (s, 2H), 5.23 (s, 2H), 5.67 (s, 2H), 7.34 (s, 1H), 7.48-7.61 (m, 2H), 7.64 (s, 1H), 8.20 (x, 1H), 8.43 (s, 1H); LC-MS (ESI): 607.1 [M+H]⁺.

### Embodiment 14 Synthesis of Compounds 1-14

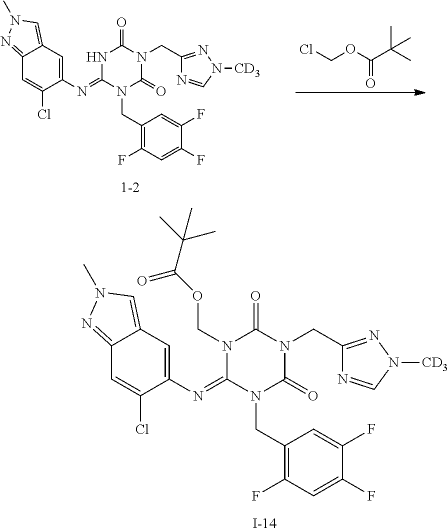

Dissolve compounds I-2 in DMF, cool in ice bath, add with sodium hydride, stir for about 30 min, then slowly add with chloromethyl tervalerate, then increase to room temperature and stir for reaction for 3 h. The reaction solution was diluted with ethyl acetate, washed with salt water, dried with anhydrous sodium sulfate, concentrated, and purified by silica gel column chromatography and compounds I-14 is obtained, H NMR (400 MHz, DMSO-d₆) 0.99 (s, 9H), 4.11 (s, 3H), 6.03 (s, 2H), 5.05 (s, 2H), 5.58 (s, 2H), 7.07 (s, 1H), 7.30-8.45 (m, 1H), 8.45-7.62 (m, 1H), 7.67 (s, 1H), 8.20 (s, 1H), 8.40 (s, 1H); LC-MS (ESI): 649.2 [M+H]⁺.

Dissolve compounds I-2 (5.2 g, 9.72 mmol) in DMF (50 mL), cool in ice bath, add sodium hydride, stir for about 30 min, then slowly added chloromethyl tervalonate (2.19 g, 14.58 mmol), then increase to room temperature and stir for reaction for 3 h. The reaction solution is diluted with ethyl acetate and washed with salt water, dried with anhydrous sodium sulfate and purified by silica gel column chromatography, then compound I-14 (1.89 g, 30% yield) is obtained.

### Embodiment 15-17

Compounds 1-15, 1-16, and 1-17 are obtained by reacting 1-3, 1-4, and 1-5 with chloromethyl acetate, respectively, using the same synthesis method as in Embodiment 6.

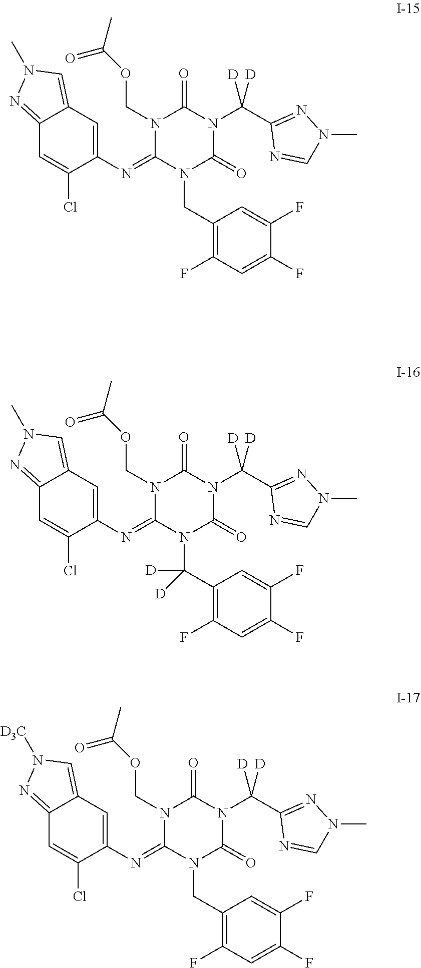

### Embodiment 18 Synthesis of Compounds 1-18

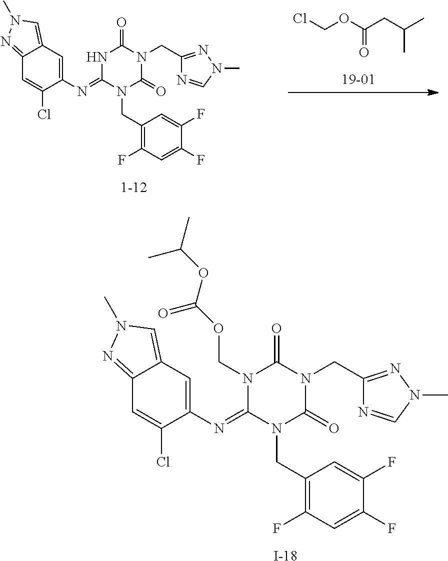

Dissolve compounds I-12 in DMF, cool in ice bath, add with sodium hydride, stir for about 30 min, then slowly add with isopropyl chloromethyl carbonate, then increase to room temperature and stir for reaction for 3 h. The reaction solution is diluted with ethyl acetate, washed with salt water, dried with anhydrous sodium sulfate, concentrated, and purified by silica gel column chromatography to obtain compound 1-18. LC-MS (ESI): 648 [M+H]⁺.

### Embodiment 19 Synthesis of Compound 1-19

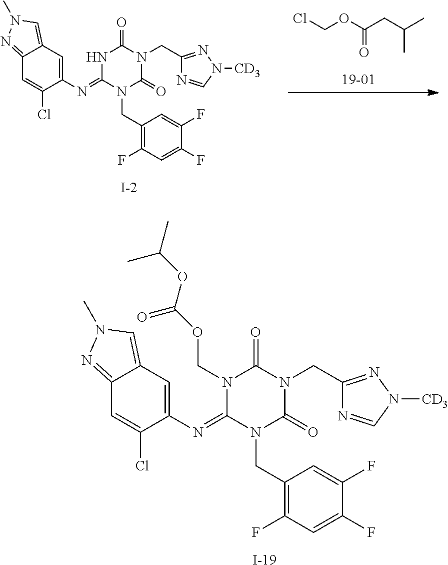

Dissolve compound I in DMF, cool in ice bath, add sodium hydride, stir for about 30 min, then slowly add isopropyl chloromethyl carbonate, increase to room temperature and stir for reaction for 3 h. The reaction solution is diluted with ethyl acetate, washed in salt water, dried with anhydrous sodium sulfate, concentrated, and purified by silica gel column chromatography to obtain compound 1-19, H NMR (400 MHz, DMSO-d₆): 1.04 (d, 6H), 4.10 (s, 3H), 4.30-4.41 (m, 1H), 5.02 (s, 4H), 7.05 (s, 1H), 7.28-7.40 (m, 1H), 7.51-7.60 (m, 1H), 7.64 (s, 1H), 8.18 (s, 1H), 8.41 (s, 1H): LC-MS (ESD): 651.2 [M+H]⁺.

Dissolve compounds I-2 (5.2 g, 9.78 mmol) in DMF, cool in ice bath, add sodium hydride, stir for about 30 min, and then slowly added isopropyl chloromethyl carbonate (2.24 g, 14.67 mmol), then increase to room temperature and stir for reaction for 3 h. The reaction solution Is diluted with ethyl acetate, washed in salt water, dried with anhydrous sodium sulfate, concentrated, and purified by silica gel column chromatography to obtain compounds 1-19 (1.78 g, 28% yield).

### Embodiments 20-23

The compounds 1-20, 1-21, 1-22, 1-23 are obtained by reacting 1-1, 1-3, 1-4, 1-5, etc., with methyl chloride acetate by the same synthesis method as in embodiment 6.

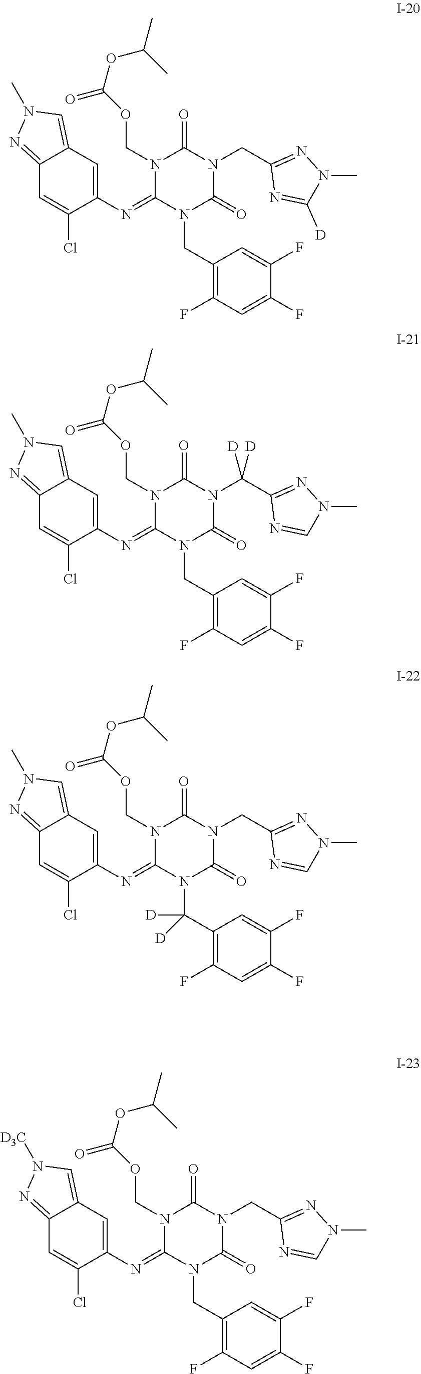

### Embodiment 24 Synthesis of Deuterated Compound 1-24

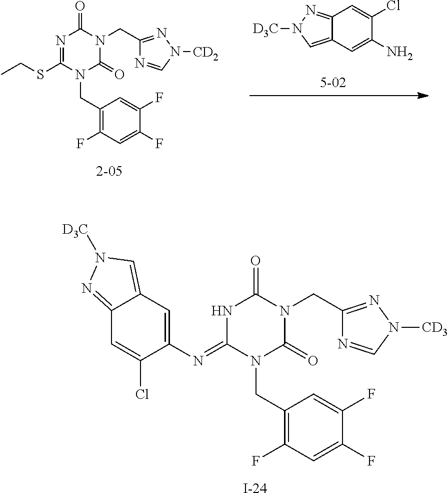

Dissolve 2-05 (5.0 g, 12.6 mmol) and 5-02 (2.06 g, 18.9 mmol) in the mixture of acetic acid (11.32 g, 189 mmol) and tert-butanol (100 mL), raise the temperature, reflux and stir it for reaction for 3 hours, cool to room temperature, and add the reaction solution to saturated sodium bicarbonate aqueous solution (500 mL). The compounds 1-24 is extracted with ethyl acetate (500 mL), combined with organic phase, washed, dried with anhydrous sodium sulfate, filtered, concentrated under reduced pressure, and the residue is isolated by silica gel column chromatography, ¹H NMR (400 MHz, DMSO-d6) 4.88 (s, 2H), 5.16 (s, 2H), 7.13 (s, 1H), 7.30-7.90 (m, 3H), 8.33 (s, 2H), 12.01 (BRS, 1H); LC-MS (ESI, m/z) 651.2 [M+H]⁺; LC-MS (ESI): 538.1 [M+H]⁺.

### Embodiment 25 Synthesis of Compound 1-25

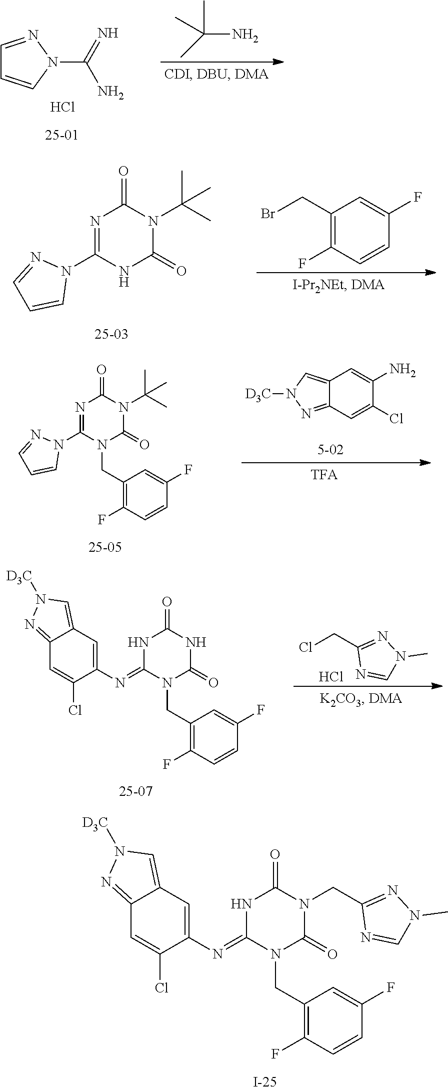

### Synthesis of Intermediate 25-03

Cooled in an ice bath, Add 1,1-bitazole CDI (170.3 g, 1.05 mmol) and DBU (167.5 g, 1.10 mmol) into the DMA solution (2 L) of tert-butylamine (73.14 g, 1.0 mol), stir for 3 h and add pyrazole-1-methylformamidine (110.12 g, 1.0 mmol) for 5 h, and then add 1,1′-carbonyl dimisazole (194.58 g, 1.2 mol) and DBU (182.7 g). 120 mmol), raise temperature to 50° C. for stirring reaction overnight (about 15 h). After cooling, the reaction liquid is poured into ice water, the pH is adjusted to 5-6 with dilute hydrochloric acid, solid matter is precipitates, filtered, washed and dried to obtain the compound 25-03, 200.1 g yield: 85%. LC-MS (ESI): 236.1 [M+H]⁺.

### Synthesis of Intermediate 25-05

Dissolve compound 25-03 (23.5 g, 100 mmol) and diisopropylethylamine (10.6 g, 120 mmol) in DMA, stir at room temperature for 30 min, and then slowly add 2,5-difluorobenyl bromide (4a, 24.75 g, 110 mmol), and then reacted at 60° C. for 8 h. TLC test reaction complete. The reaction liquid is cooled to room temperature, then pour into ice water, the pH value is adjusted to acidity with dilute hydrochloric acid 1, extracted with ethyl acetate, combined with organic phase, washed with saturated salt water, and dried with anhydrous sodium sulfate. The compound 25-05, 36.1 g is purified by silica gel column chromatography with a yield of 95.2%. LC-MS (ESI): 362.1 [M+H]⁺.

### Synthesis of Intermediate 25-07

Dissolve Compound 25-05 (7.22 g, 20.0 mmol) and 6-chloro-2-(methyl-d3)-2H-indazole-5-amine (4.43 g, 24.0 mmol) in trifluoroacetic acid (100 mL), heat to 60° C. and stir for about 5 h. After cooling to room temperature, pour the reaction liquid into ice water, solid precipitated, filtered and washed, then beaten with ethanol for 1 h, filtered and dried, the compound was 25′-07 (6.92 g, yield 82%). LC-MS (ESI): 422.1 [M+H]⁺.

### Synthesis of Compound I-25

Dissolve 25′-07 (4.21 g, 10.0 mmol) and (1-methyl-1 h-1,2, 4-triazole-3-yl) chloromethane hydro chloride (2.52 g, 15.0 mmol) in DMA (50 mL), and add anhydrous potassium carbonate (3.27 g, 23.6 mmol), rise temperature to 60° C. and stir for reaction for 4 h. Cool to room temperature, pour the reaction liquid into ice water, solid precipitated, filtered, washed in water, dried to coarse product, purified by silica gel column chromatography (gradient elution of dichloromethane/methanol), obtain I-25 (2.25 g yield 35%). ¹H NMR (400 MHz, DMSO-d₆): δ 11.10 (s, 0.5H), 9.7 (s, 0.5H), 8.39 (d, 1H), 8.31 (d, 1H), 7.75 (d, 1H), 7.67 (s, 1H), 7.34 (m, 1H), 7.26 (m, 1H), 7.17 (m, 1H), 5.28 (d, 2H), 4.92 (d, 2H), 3.80 (d, 3H); LC-MS (ESI): 644.1 [M+H]⁺.

### Embodiment 26 Synthesis of Compound 1-26

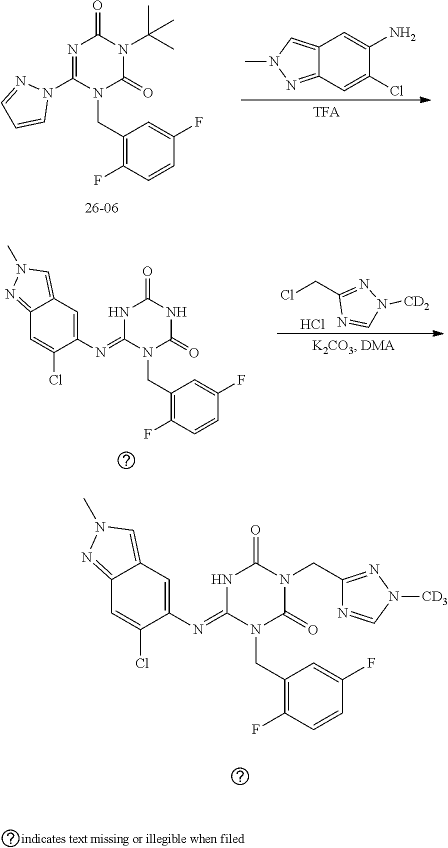

### Synthesis of Intermediate 26-01

Dissolve compound 25-06 (7.22 g, 20.0 mmol) and 6-chloro-2-methyl-2 h-indazole-5-amine (4.43 g, 24.0 mmol) in trifluoroacetic acid (100 mL), heat to 60° C. and stir for about 5 h, cool to room temperature, pour the reaction liquid into ice water, some solid precipitate, filter and wash, then beat with ethanol for 1 h, filter and dry it. Compound 26-01 is obtained. (7.11 g, yield 85%), LC-MS (ESD): 419.1 [M+H]⁺

### Synthesis of Compound I-26

Dissolve 26-01 (4.19 g, 10.0 mmol) and (1-methyl-D3-1 h-1,2, 4-triazole-3-yl) chloromethane hydrochloride (2.56 g, 15.0 mmol) in DMA (23 mL) and add anhydrous potassium carbonate (4.14 g, 30.0 mmol), rise temperature to 60° C. for stirring reaction for 6 h. Cool to room temperature, pour the reaction liquid into ammonium chloride aqueous solution, solid precipitation, filtration, water washing, drying to obtain crude products, purified by silica gel column chromatography (dichloromethane/methanol gradient elution), 1-26 is obtained (2.25 g yield 35%). ¹H NMR (400 MHz, DMSO-d₆): δ 11.11 (s, 0.5H), 9.71 (s, 0.5H), 8.38 (d, 1H), 8.31 (d (1H), 7.75 (d, 1H), 7.67 (s, 1H), 7.34 (m, 1H), 7.26 (m, 1H), 7.17 (m, 1H), 5.27 (d, 2H), 4.92 (d, 2H), 4.15 (d, 3H); L C-MS (ESI): 644.1 [M+H]⁺.

### Embodiment 27 Synthesis of Compound 1-27

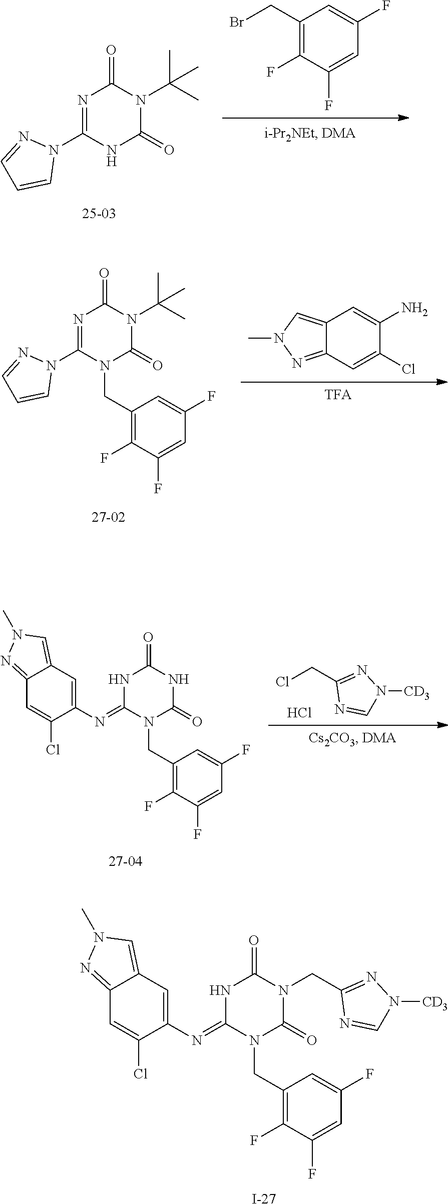

### Synthesis of Intermediate 27-02

Dissolve compound 25-03 (7.06 g, 30 mmol) and diisopropylethylamine (3.78 g, 36.0 mmol) in DMF, stir for 30 min at room temperature, then slowly add 2,3, 5-difluorobenyl bromide (7.43 g, 33.0 mmol), and then increase the temperature to 60° C. for 8 h, and the TLC test is completed. Cool the reaction liquid to room temperature, pour into ice water, the pH value is adjusted to acidity with dilute hydrochloric acid 1, extracted by ethyl acetate, combined with organic phase, washed in saturated salt water, dried with anhydrous sodium sulfate, concentrated under reduced pressure, and the crude product is purified by silica gel column chromatography to obtain compound 27-02 (10.31 g, yield 91%). LC-MS (ESI): 380.1 [M+HJ]⁺.

### Synthesis of Intermediate 27′-04

Dissolve the compound 27-02 (7.58 g, 20.0 mmol) and 6-chloro-2-methyl-2 h-indazole-5-amine (4.36 g, 24.0 mmol) in trifluoroacetic acid (100 mL) and heat to 60° C. and stir for reaction for about 5 h. After cooling to room temperature, the reaction liquid is poured into ice water, solid precipitated, filtered and washed, then beaten with ethanol 1 h, filtered and dried to obtain compound 27′-04 (6.78 g, yield 78%). LC-MS (ESI): 437.1 [M+H]⁺.

### Synthesis of Compound 1-27

Dissolve 27′-04 (4.37 g, 10.0 mmol) and (1-methyl-D3-1 h-1,2, 4-triazole-3-yl) chloromethane hydrochloride (2.57 g, 15.0 mmol) in DMA (23 mL), and add anhydrous potassium carbonate (3.27 g, 23.6 mmol), increase temperature to 60° C. and stir for reaction for 4 h, pour the reaction liquid into ammonium chloride aqueous solution, some solid precipitated, filtered, washed in water, dried to a crude product, purified by silica gel column chromatography (gradient elution of dichloromethane/methanol), and obtain I-27 (1.82 g, yield 34%). ¹H NMR (400 MHz, DMSO-d₆): δ 11.10 (brs, 0.5H), 9.70 (brs, 0.5H), 8.35 (s, 1H), 8.21 (d, 1H), 7.73 (m, 1H), 7.48 (m, 1H), 7.21 (m, 1H), 7.11 (m, 1H), 5.31 (d, 2H), 4.92 (s, 2H), 4.13 (s, 3H); L.C-MS (ESI): 535.1 [M+H]⁺.

### Embodiment 28 Synthesis of Compound 1-28

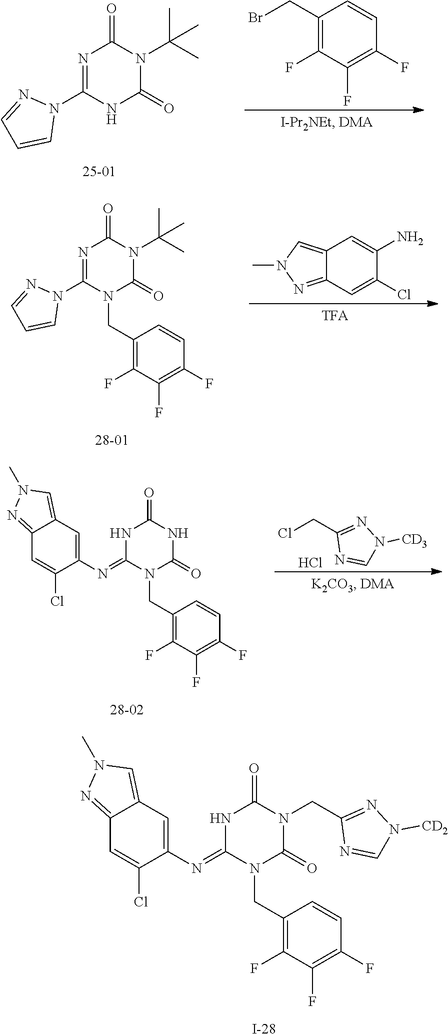

### Synthesis of Intermediate 28-01

Dissolve compound 25-01 (7.06 g, 30 mmol) and diisopropylethylamine (3.78 g, 36.0 mmol) in DMF, stir at room temperature for 30 min, and then slowly add 2,3, 4-difluorobenzyl bromide (7.43 g, 33.0 mmol), and then reacted at 60° C. for 8 h. After the reaction was completed by TLC test, the reaction liquid is cooled to room temperature, poured into ice water, the pH value was adjusted to acidity with dilute hydrochloric acid 1, extracted by ethyl acetate, combined with organic phase, washed in saturated salt water, dried with anhydrous sodium sulfate, concentrated under pressure, and the crude product is purified by silica gel column chromatography to obtain compound 28-01 (9.66 g, yield 85%). LC-MS (ESI): 380.1 [M+H]⁺.

### Synthesis of Intermediate 28-02

Dissolve compound 28-01 (7.58 g, 20.0-mmol) and 6-chloro-2-methyl-2 h-indazole-5-amine (4.36 g, 24.0 mmol) in trifluoroacetic acid (100 mL) and heat at 60° C. for about 5 h. After cooling to room temperature, the reaction liquid is poured into ice water, solid precipitated, filtered and washed, then beaten with ethanol for 1 h, filtered and dried to obtain compound 28-02 (6.54 g, yield 75%). LC-MS (ESI): 437.1 [M+H]⁺.

### Synthesis of Compound I-28

Dissolve 28-02 (4.36 g, 10.0 mmol) and (1-methyl-D3-1 h-1,2, 4-triazole-3-yl) oxymethane hydrochloride (2.57 g, 15.0 mmol) in DMA (23 mL), then add anhydrous potassium carbonate (3.27 g, 23.6 mmol), and the reaction is stirred at 60° C. for 4 h. Cool to room temperature, the reaction liquid is poured into ammonium chloride aqueous solution, solid precipitation, filtration, water was h, dried to obtain crude products, purified by silica gel column chromatography (gradient elution of dichloromethane/methanol), 1-27 (1.87 g yield 35%). ¹H NMR (400 MHz, DMSO-d₆): δ 11.04 (BRS, 0.5H), 9.64 (BRS, 0.5H), 8.33 (s, 1H), 7.19 (s, 1H), 7.63 (s, 1H), 7.30 (m, 2H), 7.15 (m, 1H), 5.26 (d, 2H), 4.88 (s, 2H), 4.12 (d, 3H); LC-MS (ESI): 535.1 [M+H]⁺.

### Embodiment 29 Synthesis of Compounds 1-29

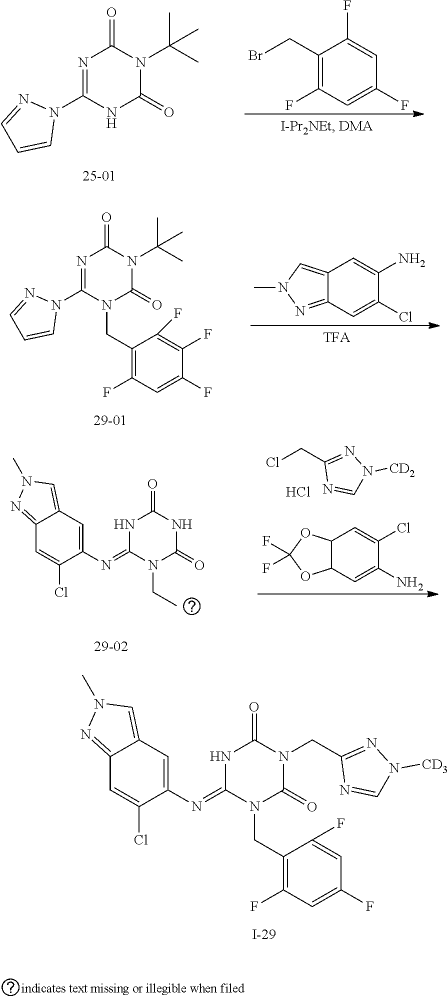

### Synthesis of Intermediate 29-01

Dissolve compounds 25-01 (7.06 g, 30 mmol) and diisopropylethylamine (3.78 g, 36.0 mmol) in DMA, stir at room temperature for 30 min, and then slowly add 2,4, 6-difluorobenzyl bromide (7.43 g, 33.0 mmol) in drops, and then reacted at 60° C. for 8 h. After the reaction is completed, cool the reaction liquid to room temperature, pour into ice water, the pH value is adjusted to acidity with dilute hydrochloric acid 1, extracted by ethyl acetate, combined with organic phase, washed with saturated salt water, dried with anhydrous sodium sulfate, concentrated under pressure, and the crude product is purified by silica gel column chromatography to obtain compound 28-01 (9.66 g, yield 85%). LC-MS (ESI): 380.1 [M+H]⁺.

### Synthesis of Intermediate 29-02

Dissolve compound 29-01 (7.58 g, 20.0 mmol) and 6-chloro-2-methyl-2 h-indazole-5-amine (4.36 g, 24.0 mmol) in trifluoroacetic acid (100 mL), heat at 60° C. and stir for about 5 h. After cooling to room temperature, pour the reaction liquid into ice water, solid precipitates, filter and wash, then beat with ethanol for 1 h, filter and dry to obtain compound 29-02 (6.54 g, yield 75%), LC-MS (ESI): 437.1 [M+H]⁺.

### Synthesis of Compound I-29

Dissolve combine 29-02 (4.36 g, 10.0 mmol) and (1-methyl-D3-111-1,2, 4-triazole-3-yl) chloromethanes hydrochloride (2.57 g. 15.0 mmol) into DMF (23 mL), add anhydrous potassium carbonate (3.27 g). 23.6 mmol), rise temperature to 60° C. and stir reaction for 4 h. Cool to room temperature, the reaction liquid is poured into ammonium chloride aqueous solution, solid precipitates, filter, water washing, dry to obtain crude products, purified by silica gel column chromatography (dichloromethane/methanol gradient elution) to get 1-27 (2.03 g yield 38%). ¹H NMR (400 MHz, DMSO-d₆): δ 11.04 (brs, 0.5H), 9.64 (BRS, 0.5H), 8.33 (s, 1H), 8.20 (s, 1H), 7.62 (s, 1H), 7.14 (m, 2H), 7.03 (m, 1H), 5.26 (d, 2H), 4.88 (s, 2H), 4.15 (d, 3H); LC-MS (ESI): 535.1 [M+H]⁺.

### Comparative Example 1: Synthesis of Example Compound I-30

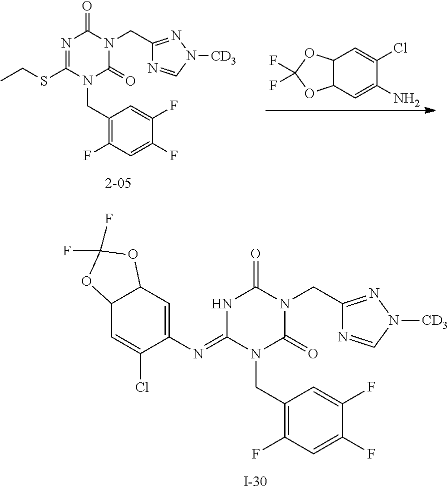

Dissolve 2-05 (5.0 g, 12.6 mmol), 2, 2-difluoro-5-amino-6-chloro-1, 3-benzodioxazole (2.06 g, 18.9 mmol) in the mixture of acetic acid (11.32 g, 189 mmol) and isoamyl alcohol (100 mL), raise the temperature, reflux and stir it for reaction for 3 hours, cool to room temperature. Add the reaction solution to saturated sodium bicarbonate aqueous solution (500 mL), extract with ethyl acetate (500 mL), combine with organic phase, wash in water, dry with anhydrous sodium sulfat e, filter, concentrate under reduced pressure, and the residue is purified by silica gel column chromatography to obtain compound 1-30, LC-MS (ESI): 563.1 [M+H]⁺. Dissolved 2-05 (4.15 g, 10.0 mmol), 6-chloro-2, 2-diamine-3a, 7a-dihydrobenzo [d][1,3]dioxacyclopentadiene-5-amine (2.06 g, 12.0 mmol) in a mixture of acetic acid (50 mL) and tert-butanol (50 mL), raise the temperature, reflux and stir it for reaction for 3 hours, cool to room temperature. Add the reaction liquid to saturated sodium bicarbonate aqueous solution (500 mL), extract with ethyl acetate (500 mL), combine with organic phase, wash in water, dry with anhydrous sodium sulfate, filter, concentrat under reduced pressure, and the residue is separated by silica gel column chromatography to obtain compound 1-30 (2.74 g, yield 49%). 1H NMR (400 MHZ, DMSO-d6): δ 11.13 (BRS, 1H), 8.37 (s, 1H), 7.45-7.65 (m, 3H), 7.07 (m, 1H), 5.14 (s, 2H), 4.92 (s, 2H); LC-MS (ESI): 561.1 [M+H]⁺.

### Embodiment 30 Preparation of the Acetic Acid Coproduct (1:2, SHEN210) of Compound I-2

Suspend and dissolve I-2 (2.14 g, 4.0 mmol) in acetic acid (10 mL), heat to 100° C. to complete dissolve, cool to room temperature (25° C.), and stir for 3 h. After filtration, water washing, ethanol washing and drying, 2.34 g solid is obtained. 1H NMR (400 MHz, Pyridine-d5) δ 8.33 (s, 1H), 7.98-7.90 (m, 1H), 7.89 (s, 1H), 7.81 (s, 1H), 7.21-7.17 (m, 2H), 5.61 (s, 2H), 5.57 (s, 2H), 3.99 (s, 3H), 2.16 (s, 6H). The powder diffraction pattern of compound I-2 acetate (XPRD, As shown in FIG. 1) at the diffraction angles of (2θ) 5.4±0.2°, 9.9±0.2°, 10.7±0.2°, 12.6±0.2°, 19.8±0.2°, 22.2±0.2°, 25.5±0.2°, 27.3±0.2°, 28.0±0.2° in the diffraction peak. In particular, diffraction peak at 5.4±0.2°, 19.8±0.2°, 22.2±0.2° and 25.5±0.2° is especially obvious.

### Embodiment 31 Preparation of Compound I-2 Fumaric Acid Coproduct (1:1, SHEN211)

Dissolve and suspend I-2 (2.14 g, 4.0 mmol) in ethyl acetate (20 mL), add fumaric acid (0.49 g, 4.2 mmol) and stir at room temperature for 1 h. Filter and dry, 2.34 g solid is obtained.

¹H NMR (400 MHz, DMSO-d6) δ 8.35 (s, 1H), 8.30 (s, 1H), 7.71 (s, 1H), 7.48-7.64 (m, 2H), 6.60 (s, 3H), 5.23 (s, 2H), 4.92 (s, 2H), 4.14 (s, 3H). ¹H NMR (400 MHz, Pyridine-d5) δ 8.33 (s, 1H), 8.00-7.92 (m, 1H), 7.90 (s, 1H), 7.81 (s, 1H), 7.44 (s, 3H), 7.30-7.13 (m, 1H), 5.61 (s, 2H), 5.57 (s, 2H), 3.99 (s, 3H).

Dissolve I-2 (2.14 g, 4.0 mmol) in ethyl acetate (20 mL), add fumaric acid (0.49 g, 4.2 mmo), raise temperature and stir at 50° C. for 3 h. Cool, filter and dry, 2.34 g solid is obtained.

I-2 fumaric acid coproduct powder diffraction pattern (XPRD, as shown in FIG. 2) has diffraction peaks at on the diffraction angle (2θ) 6.05±0.2°, 7.85±0.2°, 9.55±0.2°, 10.22±0.2°, 11.02±0.2°, 11.60±0.2°, 12.01±0.2°, 12.39±0.2°, 13.43±0.2°, 13.88±0.2°, 14.79±0.2°, 15.21±0.2°, 16.41±0.2°, 17.19±0.2°, 18.13±0.2°, 18.71±0.2°, 19.16±0.2°, 19.59±0.2°, 19.91±0.2°, 20.43±0.2°, 20.97±0.2°, 21.53±0.2°, 22.04±0.2°, 22.70±0.2°, 23.16±0.2°, 23.60±0.2°, 23.85±0.2°, 24.76±0.2°, 25.47±0.2°, 27.13±0.2°, 27.77±0.2°, 28.28±0.2°, 28.95±0.2°, 29.76±0.2°, 31.17±0.2°, 32.11±0.2°, 32.64±0.2°, 33.34 soil 0.2, 34.03±0.2° and 35.00±0.2.

Refer to embodiment S-217622 for the preparation of fumaric acid coproduct (1:1, CVL201)

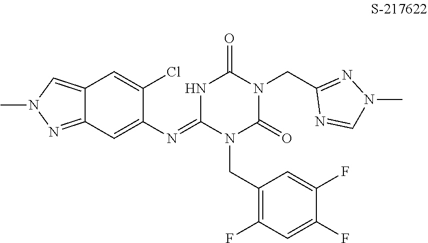

Dissolve S-217622 (2.14 g, 4.0 mmol) in ethyl acetate (20 mL), add fumaric acid (0.49 g, 4.2 mmo) and stir at room temperature for 1 h. Filtered and dried, 2.34 g solid was obtained. ¹H NMR (400 MHz, Pyridine-d5) δ 3.64 (s, 3H), 3.98 (s, 3H), 5.57 (s, 2H), 5.61 (s, 2H), 7.16-7.25 (m, 2H), 7.44 (s, 2H), 7.80 (s, 1H), 7.88 (s, 1H), 7.89-7.97 (m, 1H), 8.31 (s, 1H); Purity, 99.0% (HPLC)

Dissolve S-217622 (2.14 g, 4.0 mmol) in ethyl acetate (20 mL), add fumaric acid (0.49 g, 4.2 mmo) and stir at 50° C. for 3 h. Cool, filter and dry, then 2.34 g solid is obtained.

Embodiment 32 Preparation of the acetic acid coproduct (1:2; CVL202) of S-217622 Suspend and dissolve S-217622 (2.14 g, 4.0 mmol) in acetic acid (10 mL), heat to 100° C. to complete dissolve, and cool to room temperature (25° C.). Stir for 3 h. Filter, wash with water, wash with ethanol and dry, 2.32 g white solid is obtained. 1H NMR (400 MHz, CDCl3), δ 7.98 (s, 1H), 7.89 (BRS, 1H), 7.80 (s, 2H), 7.41 (dd, 1H), 7.07 (s, 1H), 6.92 (dd, 1H), 5.34 (s, 2H), 5.13 (s, 2H), 4.21 (s, 3H), 3.88 (s, 3H), 2.09 (s, 6H).

### Embodiment 32 Preparation of a Ternary Coproduct (1:1:1, SHEN212) of Compound I-2

Dissolve I-2 (2.14 g, 4.0 mmol) and niacinamide (0.51 g, 4.18 mmol) in ethyl acetate) with fumaric acid (0.49 g, 4.2 mmol) at room temperature for 1 h. After iltering, 2.84 gr white solid is obtained. 1H NMR (400 MHz, DMSO-d6) δ 13.12 (brs, 2H), 9.03 (s, 1H), 8.70 (d, J=4.0 Hz, 1H), 8.36 (s, 1H), 8.10-8.25 (m, 2H), 7.52-7.90 (m, 4H), 7.50 (dd, J=4.0, 8.0 Hz, 1H), 6.63 (s, 3H), 5.22 (s, 2H), 4.92 (s, 2H), 4.15 (s, 3H). 1H NMR (400 MHz, Pyridine—d5), δ 9.71 (d, 1H), 9.14 (BRS, 1H), 8.83 (dd, 1H), 8.64 (BRS, 1H), 8.59-8.53 (m, 1H), 8.32 (s, 1H), 7.99-7.90 (m, 1H), 7.89 (s, 1H), 7.81 (s, 1H), 7.44 (s, 2H), 7.36 (DDD, 1H), 7.21-7.14 (m, 1H), 5.61 (s, 2H), 5.57 (s, 2H), 3.98 (s, 3H).

Or, dissolve I-2 (2.14 g. 4.0 mmol) and niacinamide (0.51 g, 4.18 mmol) in ethyl acetate, add fumaric acid (0.49 g, 4.2 mmo), stir at 50° C. for 3 h. Cool and filter to obtain 2.84 g white solid.

Ternary coproduct powder Diffraction pattern (XPRD, as shown in FIG. 3 below) of I-2 have diffraction peaks at 2-theta angles at 10.406, 11.188, 11.772, 12.202, 12.556, 13.589, 14.075, 14.973, 15.692, 17.37, 18.212, 18.464, 18.874, 19.34, 19.752, 20.104, 20.28, 20.59, 21.154, 21.743, 22.246, 22.536, 22.868, 23.342, 23.767, 24.003, 24.92, 25.641, 25.99, 27.589, 28.038, 28.859, 29.677, 29.911, 31.356, 32.779, 33.502, 35.14, 36.251 and 39.648±0.2°.

Ternary coproduct Powder Diffraction Pattern of I-2 (XPRD, As shown in FIG. 3) on the diffraction Angle (2θ) 10.4±0.2°, 11.2±0.2°, 11.8±0.2°, 12.2±0.2°, 12.6±0.2°, 13.6±0.2°, 14.1±0.2°, 15.0±0.2°, 15.7±0.2°, 17.4±0.2°, 18.2±0.2°, 18.5±0.2°, 18.9±0.2°, 19.3±0.2°, 19.8±0.2°, 20.1±0.2°, 20.3±0.2°, 20.6±0.2°, 21.2±0.2°, 21.7±0.2°, 22.2±0.2°, 22.5±0.2°, 22.9±0.2°, 23.3±0.2°, 23.8±0.2°, 24.0±0.2°, 24.9±0.2°, 25.6±0.2°, 26.0±0.2°, 27.6±0.2°, 28.0±0.2°, 28.9±0.2°, 29.7±0.2°, 29.9±0.2°, 31.4±0.2°, 32.8±0.2°, 33.5±0.2°, 35.1±0.2°, 36.3±0.2° and 39.6±0.2° have diffraction peak.

Particularly, in the place of 11.2±0.2°, 15.7±0.2°, 18.9±0.2°, 19.3±0.2°, 22.9°+0.2°, 24.0±0.2°, 24.9±0.2°, 29.6±0.2° the diffraction peak is especially striking.

The beneficial effects of the compound of the invention are illustrated by the following effect experiments.

### Effect Embodiment 1 Inhibition Test of 3CL Protease by Some Compounds of the Invention

The fluorescence resonance energy transfer method reported in the literature is adopted (J in et al 0.2020. Structure of Mprofrom SARS-CoV-2 and discovery of its inhibitors. Nature, 582: 289-293), the enzyme inhibitory activity of the compound is determined. The catalytic activity and initial rate of 3CL enzyme are determined by enzyme kinetics using commercially available fluorescence-labeled polypeptide MCA-AVLQSGFR-Lys (Dmp)-Lys-NH2 as substrate (GLBiochem, Shanghai). The incubation system contained 3CL protease of 2019-nCoV (0.2 μM), fluorescently labeled peptides (20 μM) and a series of concentrations of compounds to be tested (0-20 μM). The fluorescence intensity of the system during incubation for 2-3 minutes is measured by enzyme marker, and the excitation wavelength and detection wavelength are 320 mm and 405 mm, respectively. According to the change rate of the initial velocity of substrate Hydrolysis catalyzed by enzyme after the addition of inhibitor, the enzyme inhibition rate of the tested substance at different concentrations was calculated. All experiments are repeated three times, and IC 50 values of inhibitory enzymes are calculated by Prism5 software. We determined the inhibitory activities of some of the compounds described in embodiments 1-29 against the novel coronavirus 3CL protease, and the specific results are shown in Table 1.

TABLE 1

| Compound number | IC50(nM) |
|---|---|
| S-217622 | 18.9 |
| I-2 | 14.9 |
| I-3 | 15.9 |
| I-4 | 14.3 |
| I-5 | 19.2 |
| I-24 | 20.1 |
| I-25 | 34 |
| I-26 | 29 |
| I-27 | 30 |
| I-28 | 18 |
| I-29 | 407 |
| I-30 | >1000 |

S-217622 is selected as the positive control drug in the enzyme inhibitory activity experiment. According to the above results, it can be seen that the inhibitory activity of some compounds in the invention, that is, the tritium substitute with specific structure in S-217622, on the 3CL proteolytic enzyme of SARS-CoV-2 novel coronavirus is equivalent to that of S-217622, and the inhibitory activity of some deuterated compounds is significantly stronger than S217622.

### Effect Embodiment 2 Evaluation of Mpro Activity of Tested Compounds Against Coronavirus In Vitro (SARS-CoV-2 Wild-Type WT, E166V Mutant and Omicron P132H Mutant Mpro Protease)

The inhibitory activity of the tested compound against the novel coronavirus Mpco protease (SARS-CoV-2 wild type WT, E166V mutant and OmicromP132H mutant Mpro protease) is detected in vitro. Ensitrelvir (i.e. CVL201, lot No. Y62200-01) served as a positive control compound for the Mpeo protease test. The compound is tested at 10 concentrations, 3 times gradient dilution, and 3 multiple pores. The initial test concentration of the tested compound is 5 μM, and the compound is diluted for 10 concentration points, 3 times gradient dilution, and 3 multiple Holes, and add to the test plate. Coronavirus. Mpro protease (WT, Omicron P132H mutant, and SARS-CoV-2 E166V mutant) is added to the experimental plate containing the compound, and pre-incubated at room temperature for 30 minutes, and then reacted with the reaction substrate at 30° C. for 60 minutes. The negative controlHole, which contains enzymes and substrates but no compounds, serves as a control without inhibition. The positive controlHole containing substrate, enzyme and high concentration of positive control compound is used as the 100% inhibition control. The inhibitory activity of compounds against coronavirus Mpro protease is analyzed and calculated by GraphPad Prism software, and the results are shown in Table 2.

TABLE 2

| | Enzymatic Determination of IC50 | | |
|---|---|---|---|
| | (nM) of compounds by Mpro | | |
| | | SARS- | SARS-COV-2 |
| Compound | SARS- | COV-2 WT | Omicron |
| number | COV-2 WT | E166V mutant | P132Hmutant |
| SHEN211 | 25 | 1354 | 51 |
| CVL201 | 27 | 1467 | 55 |
| PF-07321332 | 18 | 36730 | 30 |

The results show that SHEN211 have broad-spectrum coronavirus Mpro protease inhibitory activity, which is similar to CVL201. In addition, the 3CL protein resistance mutation E166V against Nematavir (PF-07321332) also shows enzyme inhibitory activity, which is far better than Ferri Nematavir, more than 27 times. It is suggested that SHEN211 has the potential of effective treatment of nematovir resistance.

### Effect Embodiment 3: Mpro Activity of the Tested Compound Against Other Human Coronaviruses (6 Strains of Virus Mpro, 1 Experiment)

The inhibitory activity of the tested compounds against coronavirus Mpro protease is detected by enzyme assay in vitro. Test for Mpro protease can be seen in Table 3. Ensitrelvir and PF-07321332 are used as positive control compounds for Mpro protease tests. The compound is tested at 10 concentrations, 3 times gradient dilution, and 3 multiple pores. 10. The initial test cioncentration of the tested compound s 30 uM. The compound will be diluted at 10 concentration points, with a triple gradient dilution. The coronavirus Mpro protease (Table 3) is added to the experimental plate containing the compound, which is incubated at room temperature for 30 minutes, and then the reaction substrate is added for 60 minutes at 30° C. The negative control Hole contained the enzyme and substrate but do not contain the compound, as the control without inhibition. The positive controlHole contains substrates, enzymes, and a high concentration of positive control compounds as a 100% inhibition control. Fluorescent readings are detected with a multifunctional ELISA reading board. The inhibitory activity of compounds against corona virus Mpro protease is analyzed and calculated by GraphPad Prism software, and the results are shown in Table 3.

TABLE 3

| Compound | Enzymatic Determination of IC50 (μM) of compound by Mpro | | | | | |
|---|---|---|---|---|---|---|
| number | SARS | MERS | 229E | OC43 | HKU1 | NL63 |
| SHEN211 | 0.045 | 4.671 | 6010 | 0.133 | 0.038 | 25.98 |
| CVL201 | 0.043 | 4959 | 5.627 | 0.134 | 0.044 | 27.79 |
| GC-376 | 0.040 | 0.274 | 0.100 | 0.042 | 0.022 | 0.107 |

The results suggest that SHEN211 has broad-spectrum anti-coronavirus activity, which is similar to CVL201 (S-217622 fumaric acid coproduct) and has inhibitory activity against other hu man coronaviruses except novel coronavirus.

### Effect Embodiment 4 Evaluation of the Anti-SARS-CoV-2 Activity of the Tested Compound (1 Replicator Model, 2 Conditions (with or without Physiological Concentration of HSA+AAG), 1 Experiment)

The in vitro anti-SARS-CoV-2 activity of the tested compounds is evaluated using the SAR S-CoV-2 replicator model, with Remdesivir and EIDD-1931 as control compounds. The activity of the tested compounds is evaluated in the presence of HSA and AAG without or with biological concentrations. The compound is tested at 8 concentrations, 3 times gradient dilution, and 3 times multiple pores. After the initial test concentration of the tested compound is 1μ MSARS-CoV-2 replicon RNA is electrocuted into Huh7 cells and inoculated into microplates containing the compound with double dilution at a certain density. HPE control is set up (cells with SARS-CoV-2 replicons transferred by electric transmission without chemical compound treatment), and the cells are cultured in 5% CO2 and 37° C. for 1 day, and the number of GFP expressing cells in each well is detected. The cytotoxicity test is the same as the antiviral test. Cell viability is measured using cell viability assay kit CellTiter Glo (Promega). The antiviral activity and cytotoxicity of the compound are calculated by the effect of the compound at different concentrations on the changes in pseudoviral reporter gene expression and cell viability, respectively. The neutralization activity and cell viability of the samples are analyzed using GraphPad Prism with nonlinear fitting, and the EC50 and CC50 values of the compounds are calculated in the presence of HSA and AAG without or with biological concentrations, as shown in Table 4.

TABLE 4

| | | | HSA (45 mg/mL) + | | |
|---|---|---|---|---|---|
| | NoHSA + AAG | | AAG (0.7 mg/mL) | | |
| Compound | EC50 | CC50 | EC50 | CC50 | EC50 |
| number | (microns) | (microns) | (microns) | (microns) | Shift |
| SHEN211 | 0.054 | >1 | 0.874 | >1 | 16.33 |
| Remdesivir | 0.0076 | >1 | 0.123 | >1 | 16.29 |
| EIDD-1931 | 1.578 | >10 | 1437 | >10 | 0.91 |
| CVL201 | 0.051 | >1 | 0.877 | >1 | 17.13 |

The results show that in the presence of biological concentration HAS+AAG, the antiviral activity of SHEN211 and CVL201 decreased by 16 times and 17 times, which are similar.

### Effect Embodiments 5 Evaluation of Cytotoxicity of Tested Compounds (9 Cell Lines, 1 Experiment)

Nine cell lines originated from different human tissues (Table 2) are selected to evaluate the cytotoxicity of the tested compounds. Sorafenib or Taxol are used as cytotoxic control compounds. The cells and compounds are added to the 96-well test plate. Cells are cultured in culture medium containing 10% FBS, 5% CO2 and 37° C. for 3 days, and then cell viability was detected by CellTiter-Glo reagent. The raw data are used for compound cytotoxicity calculations. The dose-response curves of the compounds are analyzed by GraphPad Prism software and CC50 values are calculated. The compound will be tested at 8 concentration points, 3-fold series dilution, and 3-compound pores. The initial test concentration of the tested compound is 100 μM, and the results are shown in Table 5.

TABLE 5

| Compound | CC50(uM) | | | | | | | | |
|---|---|---|---|---|---|---|---|---|---|
| knitting | Colo- | | | | | | | | CCRF- |
| No. | 205 | HepG2 | HEK293 | MRC-5 | A375 | Molt-4 | HEp2 | HIHeLa | CEM |
| CVL201 | >100 | >100 | >100 | >100 | >100 | >100 | >100 | >100 | >100 |
| SHEN211 | >100 | >100 | >100 | >100 | >100 | >100 | >100 | >100 | >100 |
| Sorafenib | 8.725 | 5.324 | 5.578 | 13.82 | ND | ND | ND | ND | ND |
| Pacitaxel | ND | ND | ND | ND | 0.0089 | 0.0027 | 0.030 | 0.020 | 0.0034 |

The results shows that SHEN211 had no cytotoxic problem to 9 kinds of human cells.

### Effect Example 6 Mitochondrial Toxicity Evaluation of the Tested Compound (HepG2 Glucose/Galactose Test, 1 Experiment)

In vitro mitochondrial toxicity of the tested samples will be determined by HepG2 glucose/galactose assay. The tested compound is SHEN211. Rotenone as a control compound. The compound is tested at 9 concentrations, 3 times gradient dilution, and 3 multiple pores. The initial concentration of tested compounds is 500 μM. HepG2 cells cultured with glucose and HepG2 cells cultured with galactose are inoculated in microplates at a certain density and cultured overnight in a 5% CO2 incubator at 37° C. The next day, the compound is added with a double dilution ratio. Cell control group and compound test group are set up. The cells are cultured in a 5% CO2 incubator at 37° C. for 24 h. Cell viability is measured using CellTiter Glo, a cell activity assay, and the raw data are used to evaluate the mitochondrial toxicity of the compounds. The dose-response curve of the compound is analyzed by GraphPad Prism software and the CC50 value is calculated. The results are shown in Table 6.

TABLE 6

| | G glucose culture | G galactose culture | R ratio |
|---|---|---|---|
| Compound | HepG2 | HepG2 | (CC50 Glu/ |
| number | Ab CC50(μM) | Ab CC50(μM) | CC50 Gal) |
| SHEN211 | 202.33 | 206.28 | 0.98 * |
| Rotenone | 2.33 | 0.03 | 91.7 |

The results show that the mitochondrial toxicity of SHEN211 to human HepG2 cells is more than 200 μM. The difference between the CC50 and EC50 (0.084-0.210 μM) of SARS-CoV-2 strain is more than 476 times, suggesting that there is no problem of mitochondrial toxicity to human.

### Effect Example 7 Study on Liver Particle Metabolism of Some Compounds of the Invention

Kunming mouse liver microsomes (IPHASE/Huizhi taikang) are prepared by ultrafast centrifugal method. Fresh mouse liver is weighed, crushed in Tris-HCl buffer liquid with 3 times the volume, and then Homogenized with Homogenizer. The above operations are all carried out in an ice bath below 4° C., and the Homogenate is centrifugated at 7000 g and 4° C. for 20 minutes. The upper suspension is taken at 10000 g and centrifuged at 4° C. for 30 min. The supernatant is discarded and precipitated into mouse liver microsomes, which are prepared into suspension in 0.25 mol/L sucrose solution and preserved in liquid nitrogen. The protein content of mouse liver microsome is 7.8 mg/mL by Lowry method.

Mouse liver microsomes are composed of a warm incubation system in vitro. The fin al volume of the warm incubation system is 5 ml, containing mouse liver microsomes 2.0 mg/mL, glucose 6-phosphate 0.01 mmol/mL, G6-PDH 1 U/mL, magnesium chloride 4.0 umol/mL and NADPO.5 umol per mL, NADH 1. Oumol/mL is mixed and shaken well, and then oscillated in a water bath at 37° C., two parts of each sample are prepared, and the tested substance is added to the mouse liver microsome enzyme incubation solution, so that the concentration of the tested substance is 50 mg/L, fully oscillated, and incubated at 37° C. At the same time, blank control experiments are performed with the heated inactivated liver Homogenate. Oxygen is given to the surface of the incubation solution every 0.5 hour for 1 minute, and 0.5 mL of the sample is taken at 0, 5, 15, 30 and 60 minutes, respectively.

At that time, 3 times the volume of acetonitrile is added to terminate the metabolic reaction, and the metabolic clearance rate and half-life are calculated, as shown in Table 7.

TABLE 7

| Compound | | | Tic | Cla | Clape | Cl | |
|---|---|---|---|---|---|---|---|
| number | species | k | (min) | (ml/min/mg) | (ml/min/kg) | (mL/min/kg) | Eₕ(%) |
| S-217622 | rat | −0.0002 | ∞ | 0.0000 | 0.0 | 0.0 | 0.0 |
| I-2 | rat | −0.0012 | ∞ | 0.0000 | 0.0 | 0.0 | 0.0 |
| I-3 | rat | −0.0002 | ∞ | 0.0000 | 0.0 | 0.0 | 0.0 |
| I-5 | rat | −0.0005 | ∞ | 0.0000 | 0.0 | 0.0 | 0.0 |
| Testosterone | rat | 1.2226 | 0.6 | 6.1128 | 11003.1 | 54.7 | 99.5 |

According to the metabolic test results of mouse liver particles, it can be seen that deuterium-substituted triazine derivatives at different positions of the invention are basically not metabolized in mouse liver particles, and there is no significant difference compared with S-217622.

### Effect Example 7 Study on Hepatocyte Metabolism of Some Compound of the Invention

### 1. Preparation of Solution

The compound to be tested is configured as a DMSO reserve solution at a concentration of 10 mM, and then the reserve solution of the compound to be tested is diluted to a 200 uM solution with acetonitrile.

### 2. Liver Cell Body Incubation

A incubation mixture with a total volume of 200 uL is prepared with the following final component concentrations: william's E medium, liver cells (1 million/mL) and test compound or positive control (0.5 μM). After pre-incubating all other components in a 37° C.±5% CO2 incubator for 10 minutes, the compound is added. Mix with a pipette to obtain a uniform suspension, and immediately transfer the sample incubated at 20 μL for 0 minutes into the Hole of the “quenched” plate, then mix with a pipette. At 15, 30, 60, 120, and 240 minutes, the cultures are mixed with pipettes, and at each time point samples of the 20 L culture are successively transferred to Holes in separate “quenched” plates, which are then mixed with pipettes. 200 μL acetonitrile containing IS is added to the “quenched” plate, and the results are shown in Table 8.

TABLE 8

| | | | S-scale factor |
|---|---|---|---|
| | L Liver weight | Liver blood flow | (million cells/ |
| S species | (g/kg BW) | (mL/min/kg) | g liver wt.) |
| rat | 40 | 55 | 117 |
| H people | 26 | 21 | 139 |

### 3. Sample Analysis

Centrifuge the 96-well plate at 4000 rpm for 10 minutes. The 50 uL supernatant is mixed with 50 uL deionized water and then injected into the LC-MS/MS system for analysis. After data processing, parameters such as CIᵢₙₜ, CIₐₚₚ, CIₕ and Eh, etc. are calculated, and the results are shown in Table 9.

TABLE 9

| Compound | | | | Clᵢₙₜ | Clₐₚₚ | Clₕ | |
|---|---|---|---|---|---|---|---|
| number | species | k | T_{1/2}(min) | (mL/min/million cell) | (mL/min) | (mL/min) | Eₕ(%) |
| S-217622 | rat | 0.0008 | 888.1 | 0.0008 | 3.7 | 3.4 | 6.2 |
| | people | 0.0001 | 5909.4 | 0.0001 | 0.4 | 0.4 | 2.0 |
| I-2 | rat | 0.0001 | 5337.3 | 0.0001 | 0.6 | 0.6 | 1.1 |
| | people | 0.0006 | 0 | 0.0000 | 0.0 | 0.0 | 0.0 |
| I-3 | rat | 0.0014 | 496.7 | 0.0014 | 6.5 | 5.8 | 10.6 |
| | people | 0.0001 | 12226.5 | 0.0001 | 0.2 | 0.2 | 1.0 |
| I-5 | rat | 0.0008 | 833.5 | 0.0008 | 3.9 | 3.6 | 6.6 |
| | people | 0.0004 | 1794.4 | 0.0004 | 1.4 | 13 | 6.2 |

According to the results of liver cell metabolism test, it can be seen that compared with S-217622, the metabolic clearance rate of the deuterated triazine derivative in different positions of the invention is significantly reduced, and the metabolic half-life of S-217622 is prolonged after deuteration. Based on maintaining the effectiveness against SARS-CoV-2, the metabolic half-life of S-217622 is prolonged. The half-life is significantly prolonged, the demand for dosage is reduced, side effects are reduced, and the therapeutic window range is expanded. Therefore, the invention has a very good prospect for making drugs for treating diseases related to coronavirus infection.

### Effect Embodiment 9 In Vivo Pharmacokinetic Study of Some Compound of the Invention

### 1. Preparation of Test Products

IV: 5% DMSO+95% PG
PO: 5% DMSO+95% MC (0.5%)
G10: CVL201: nicotinamide mass ratio is 5.3:1, for example, weigh 6.09 mg (5*1.218) CVL201, then weigh 1.149 mg (6.09 mg/5.3) nicotinamide, and then use 5 ml of solvent to prepare a suspension.

### 2. Experimental Animals

Species: Healthy male SD (Sprague Dawley) rats (SPF class), weighing 180-220 g.
Source: Laboratory Animal Management Department, Shanghai Institute of Family Planning Science, animals were transferred from animal reserve of experimental institutions (999M-017).
Number: 30 males
Animal selection: No random grouping

### 3. Drug Administration and Blood Collection Time

Weigh before dosing, calculate dosage according to body weight. The drug is administered intravenously or by appetite irrigation.

The drug is administrated by intragastric administration (5 mg/kg), and 0.2 ml of blood is collected from the jugular vein of rats at 0.25 h, 0.5 h, 1 h, 2 h, 4 h, 6 h, 8 h and 24 h after administration, and plasma is separated and prepared. The concentration of compounds in plasma is determined by LC-MS/MS.

The drug is administered intravenously (0.5 mg/kg), and 0.2 ml of blood is collected from the jugular vein of rats at 0.033 h, 0.25 h, 0.5 h, 1 h, 2 h, 4 h, 8 h, and 24 h after administration, with EDTA-K2 anticoagulant and placed on ice.

### 5. Plasma Sample Treatment

The blood samples are collected and placed on ice and the plasma is centrifuged within 1 hour (centrifugation condition: 6800 g, 6 minutes, 2-8° C.). Plasma samples are stored in −80° C. refrigerator before analysis.

### 6. Sample Analysis

### 1) Sample Preparation for LC-MS/MS Determination:

S-217622 and 1-5: Protein precipitation of 40 μL plasma sample with 400 μL methanol containing 10 ng/mLIS (IS Verapamil, Verapamil), swirl mixture for 1 min, then centrifuge at 18000 g for 7 min. The 400 μL supernatant is transferred to the 96-well plate. LC-MS/MS analysis is performed with 1 μL supernatant.

1-2: Protein precipitation is performed on a 20 uL plasma sample with 400 μL methanol containing 10 ng/mL IS (IS is Verapamil), the mixture is vortex for 1 min, then centrifuged at 18000 g for 7 min, and 300 μL supernatant is transferred to a 96-well plate. LC-MS/MS analysis is performed with 8 μL supernatant.

### 2) LC-MS/MS Analysis Method:

Mobile phase A: 0.1% formic acid aqueous solution.
Mobile phase B: 0.1% ethyl formate solution:
Pillar: ACQUITY UPLC BEH C18 1.7 μm 2.1*50 mm:
Flow rate: 0.60 mL/min.
Column temperature: 40° C.

The gradient elution procedure is shown in Table 10:

TABLE 10

| S-217622 | | I-2 | | I-24 | |
|---|---|---|---|---|---|
| | M Mobile | | M Mobile | | M Mobile |
| T time | phase | T time | phase | T time | phase |
| (min) | B (%) | (min) | B (%) | (min) | B (%) |
| 1 Initial | 10 | 0.01 | 10 | Initial | 10 |
| value | | | | | |
| 0.60 | 90 | 0.60 | 90 | 0.60 | 90 |
| 1.20 | 90 | 1.10 | 90 | 1.20 | 90 |
| 1.21 | 10 | 1.11 | 10 | 1.21 | 10 |
| 1.50 | 10 | 1.40 | Stop | 1.50 | 10 |

S-217622 peak time: 0.69 min: MS: Q1/Q3 Masses: 532.10394.00 Da
   Peak time: 0.63 min MS: Q1/Q3 Masses: 455.30/165.20 Da
I-2 Peak time: 0.86 min: MS: Q1/Q3 Masses: 535.108286.00 Da
   Peak time marked inside: 0.82 min: MS: Q1/Q3 Masscs: 455.30165.20 Da
1-24 Peak time: 0.69 min; MS: Q1/Q3 Masses: 538.103285.90 Da
   Peak time: 0.63 min: MS: Q1/Q3 Masses: 455.304165.20 Da.

### 3) Result Analysis

Through the blood concentration data at different time points, the pharmacokinetic parameters are calculated by Phocnix WinNonlin7.0 non-atrioventricular model, and the parameters such as AUC_{0-∞}, Cₘₐₓ, Tₘₐₓ and T_{1/2} and their mean values and standard deviations are provided. The results are shown in FIG. 11 below.

TABLE 11

| | | Administration | | | | |
|---|---|---|---|---|---|---|
| | administration | dose | T_{1/2} | Tₘₐₓ | Cₘₐₓ | AUC_{0-∞} |
| compound | route | (mg/kg) | (h) | (h) | (ng/ml) | (h*ng/ml) |
| S-217622 | IV | 0.5 | 2.70 | 0.03 | 2307.23 | 4516.78 |
| S-217622 | PO | 5.0 | 2.80 | 3.33 | 4156.41 | 45240.04 |
| CVL201 | PO | 5.0 | 2.89 | 4.67 | 4516.47 | 53851.04 |
| (S-217622 fumaric acid | | | | | | |
| coproduct) | | | | | | |
| CVL201 + nicotinamide | PO | 5.0 | 2.64 | 3.00 | 4851.48 | 49816.19 |
| I-2 | IV | 0.5 | 3.36 | 0.03 | 2882.80 | 7943.98 |
| I- 2 | PO | 5.0 | 3.04 | 2.33 | 6019.80 | 65325.11 |
| SHEN211 | PO | 5.0 | 3.22 | 2.00 | 4710.93 | 45466.17 |
| (I-2 fumaric acid | | | | | | |
| coproduct) | | | | | | |
| SHEN212 (Ternary | PO | 5.0 | 3.19 | 3.67 | 5969.50 | 62126.14 |
| products) | | | | | | |
| (I-2 ternary coproduct) | | | | | | |
| 1-19 (Prodrug) | PO | 5.0 | 2.91 | 3.33 | 6182.77 | 70850.41 |
| 1-24 PO | PO | 5.0 | 3.51 | 4.00 | 5211.96 | 58251.56 |

The experimental results show that compared with S-217622, deuterium compound I-2 provide 125% Cₘₐₓ, increases 198% AUC, extends 124% T_{1/2}, increases 145% Cₘₐₓ after oral administration, increases 144% AUC, and increases 149% Cₘₐₓ after prodrug modification, the AUC increased by 156%. Compared with S-217 fumaric acid eutectic group, Cₘₐₓ and AUC increased by 132% and 115% after administration of 1-2 ternary coproduct PO, Cₘₐₓ increased by 123% and AUC increased by 124% after administration of 1-2 ternary coproduct PO compared with S-217622 fumaric acid coproduct combined with niacinamide group. T_{1/2} is extended by 121%. It can be seen that the deuterium modification I-2 of S-217622 can significantly increase blood drug concentration and prolong metabolic half-life on the basis of retaining the effectiveness against SARS-CoV-2, which helps to reduce the used dose, reduce side effects, and expand the therapeutic window. Therefore, the invention has a very good prospect for preparing drugs for treating diseases related to corona virus infection.

The above embodiments are only examples for clear illustration and are not limitations on embodiments. For ordinary technicians in the field, other changes or changes in different forms can be made on the basis of the above description. It is not necessary and cannot be exhaustive here. The obvious changes or alterations induced by this mountain are still within the scope of protection of the invention.

Although the specific embodiments of the invention are described above, it should be understood by those skilled in the art that these embodiments are only examples and that a variety of changes or modifications may be made to them without deviating from the principle and substance of the invention. Therefore, the scope of protection of the invention is limited by the attached claims.

1. A triazine derivative shown in formula (I′) or a pharmaceutically acceptable salt thereof, 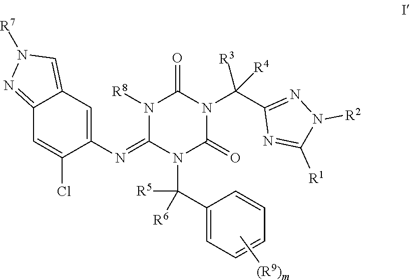
Where R¹ is Hydrogen or deuterium.
R2 is a methyl group, or a methyl group replaced by 1, 2 or 3 tritium;
R³ is hydrogen or deuterium,
R⁴ is hydrogen or deuterium,
R⁵ is hydrogen or deuterium,
R⁶ is hydrogen or deuterium,
R⁷ is a methyl group or a methyl group replaced by 1, 2, or 3 tritium,
R8 is Hydrogen, 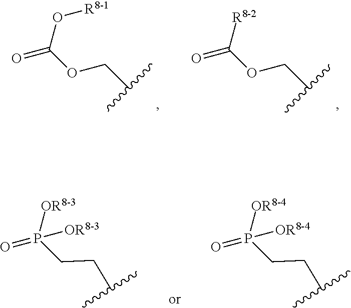
R⁸⁻¹ is C₁-C₁₀ alkyl,
R⁸⁻² is C₁-C₁₀ alkyl,
Each R⁸⁻³ is independently hydrogen, C₁-C₁₀ alkyl, or sodium,
Each R⁸⁻⁴ is independently hydrogen, C₁-C₁₀ alkyl, or sodium,
Each R9 is independently hydrogen or halogen,
m is 2, 3, 4 or 5.
Moreover, the compound shown in formula I′ satisfies one or both of the following conditions:
(1) R¹, R², R³, R⁴, R¹, R⁶ and R⁷ contain at least one deuterium:
(2) R8 is

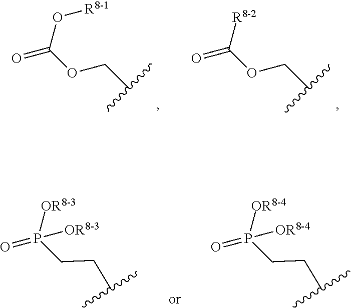

2. A triazine derivative, or a pharmaceutically acceptable salt thereof, as described in formula (I′) of claim 1, is characterized by the fact that a triazine derivative as described in formula (I′) satisfies one or more of the following conditions:
(1) In R⁸⁻¹, the mentioned C₁˜C₁₀ alkyl group is C₁˜C₆ alkyl groups, preferably methyl, ethyl, n-propyl, isopropyl, n-butyl, isobutyl, sec-butyl or tert-butyl groups, such as methyl, ethyl, isopropyl.
(2) In R⁸⁻², the mentioned C₁˜C₁₀ alkyl group is C₁˜C₆ alkyl groups, preferably methyl, ethyl, n-propyl, isopropyl, n-butyl, isobutyl, sec-butyl or tert-butyl, such as methyl, ethyl, isopropyl, n-butyl or tert-butyl.
(3) In R⁸⁻³, the mentioned C₁˜C₁₀ alkyl group is C₁˜C₆ alkyl groups, preferably methyl, ethyl, n-propyl, isopropyl, n-butyl, isobutyl, sec-butyl or tert-butyl.
(4) In R⁸⁻⁴, the mentioned C₁˜C₁₀ alkyl group is C₁˜C₆ alkyl groups, preferably methyl, ethyl, n-propyl, isopropyl, n-butyl, isobutyl, sec-butyl or tert-butyl.
(5) When R⁸ is Hydrogen, the three classes of derivatives shown in the formula (I′) may also be tautomers of them, e.g. 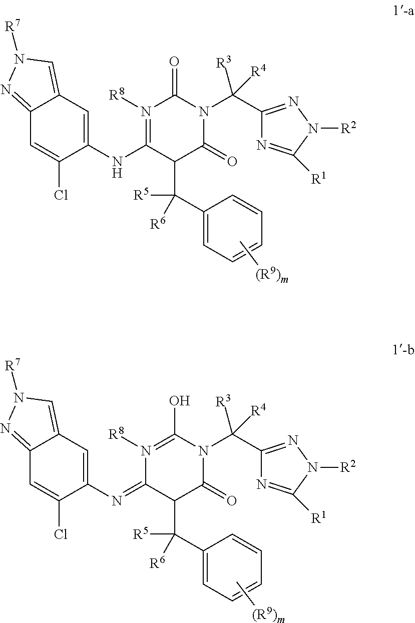
And (6) R⁹, the Halogens are neon, chlorine, bromine, or iodine, such as fluorine.

3. A triazine derivative, or a pharmaceutically acceptable salt thereof, as shown in formula I′) of claim 1, is characterized by the fact that a triazine derivative as shown in formula (I′) satisfies one or more of the following conditions:
(1) R² is methyl or —CD₃.
(2) R⁷ is methyl or —CD₃.
(3) R⁸⁻¹ is C₁-C₆ alkyl.
(4) R⁸⁻² is C₁-C₆ alkyl.
(5) R⁸⁻³ is sodium.
(6) R⁸⁻⁴ is sodium.
(7) both R³ and R⁴ are deuterium.
(8) both R⁵ and R⁶ are deuterium.
(9) R⁹ is fluorine:
And (10) m is 2 or 3.

4. A triazine derivative or a pharmaceutically acceptable salt thereof as indicated in formula (I) of claim 1 is characterized by the fact that the triazine derivative as indicated in formula (I) satisfies one or both of the following conditions.
(1) R⁸ is Hydrogen, 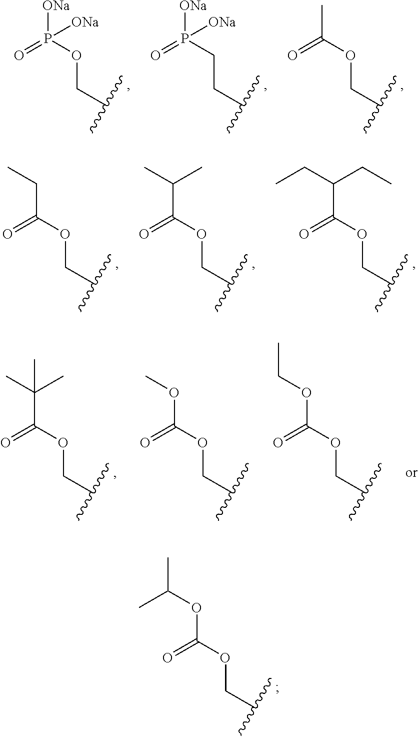
and (2) 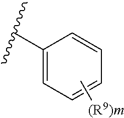
is

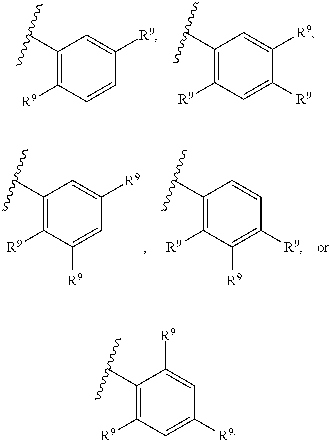

5. A triazine derivative or a pharmaceutically acceptable salt thereof as indicated in formula (I′) of claim 1, characterized that the triazine derivative as indicated in formula (I′) is option 1, option 2, option 3, option 4, option 5 or option 6,
Option 1.
R¹ is deuterium, R² is methyl, R³ is Hydrogen, R⁴ is Hydrogen, R5 is Hydrogen. R⁶ is Hydrogen,
R⁷ is methyl group, R8 is Hydrogen, 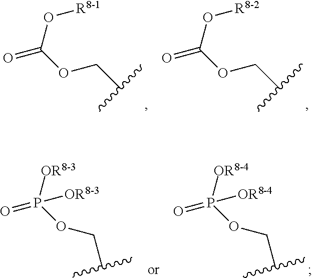
R⁸⁻¹ is C₁-C₆ alkyl, R⁸⁻² is C₁-C₆ alkyl, Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen, m is 2, 3, 4, or 5.
Option 2
R¹ is deuterium and R² is —CD₃; R³ is hydrogen; R⁴ is hydrogen; R⁵ is hydrogen; R⁶ is hydrogen;
R⁷ is
methyl or —CD₃; R⁸ is hydrogen, 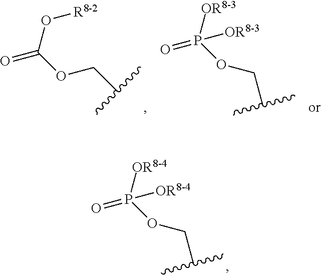
R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen, m is 2, 3, 4, or 5.
Better, in one scheme, R¹ is deuterium and R² is —CD₃; R³ is hydrogen; R⁴ is hydrogen;
R⁵ is hydrogen; R⁶ is hydrogen; R⁷ is methyl or —CD₃; R⁸ is hydrogen, 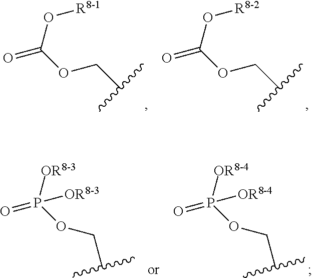
R8-1 is C1-C6 alkyl, R8-2 is C1-C6 alkyl. Each R8-3 is independently hydrogen, C1-C6 alkyl or sodium, Each R8-4 is independently hydrogen, C1-C6 alkyl or sodium. R 9 is fluorine, 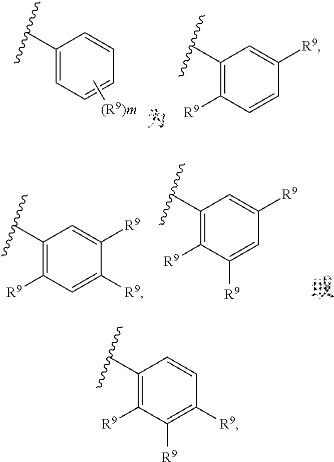
Option 3
R¹ is hydrogen and R² is methyl; R³ is tritium; R⁴ is tritium; R⁵ is hydrogen; R⁶ is hydrogen; R⁷ is methyl; R⁸ is hydrogen, 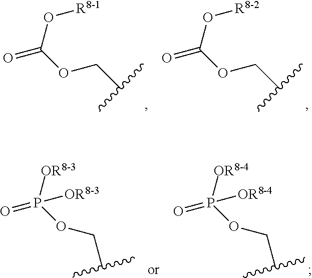
R⁸⁻¹ is C₁-C₆ alkyl, R⁸⁻² is C₁-C₆ alkyl. Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen. m is 2, 3, 4, or 5.
Option 4
R¹ is hydrogen and R² is methyl; R³ is hydrogen; R⁴ is hydrogen; R⁵ is tritium; R⁶ is tritium; R⁷ is methyl; R⁸ is hydrogen, 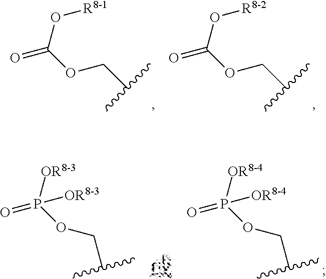
R8-1 is C1-C6 alkyl, R8-2 is C1-C6 alkyl. Each R8-3 is independently hydrogen, C1-C6 alkyl or sodium, Each R8-4 is independently hydrogen, C1-C6 alkyl or sodium. each R9 is independently hydrogen or halogen. m is 2, 3, 4, or 5.
Option 5
R¹ is hydrogen and R² is methyl or —CD₃; R³ is hydrogen; R⁴ is hydrogen; R⁵ is hydrogen; R⁶ is hydrogen; R⁷ is —CD₃; R⁸ is hydrogen, 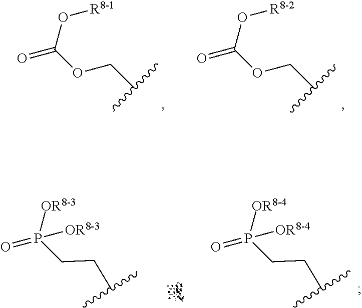
R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen, m is 2, 3, 4, or 5.
Option 6
R¹ is hydrogen or deuterium, R² is methyl or —CD₃; R³ is hydrogen or deuterium; R⁴ is hydrogen or deuterium; R⁵ is hydrogen or deuterium; R⁶ is hydrogen or deuterium; R⁷ is methyl or —CD₃; R⁸ is 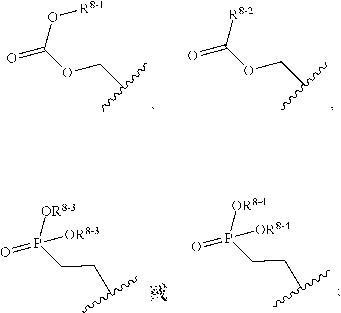
R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium. each R⁹ is independently hydrogen or halogen, m is 2, 3, 4, or 5.
Better, R¹ is hydrogen or deuterium, R² is methyl or —CD₃; R³ is hydrogen or deuterium; R⁴ is hydrogen or deuterium; R⁵ is hydrogen or deuterium; R⁶ is hydrogen or deuterium; R⁷ is methyl or —CD₃; R⁸ is 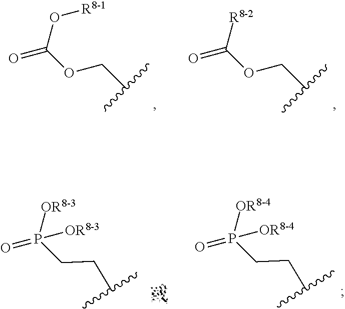
R⁸⁻¹ is C₁-C₆ alkyl, R⁸⁻² is C₁-C₆ alkyl.
Each R⁸⁻³ is independently hydrogen, C₁-C₆ alkyl or sodium, Each R⁸⁻⁴ is independently hydrogen, C₁-C₆ alkyl or sodium, R⁹ is fluorine,

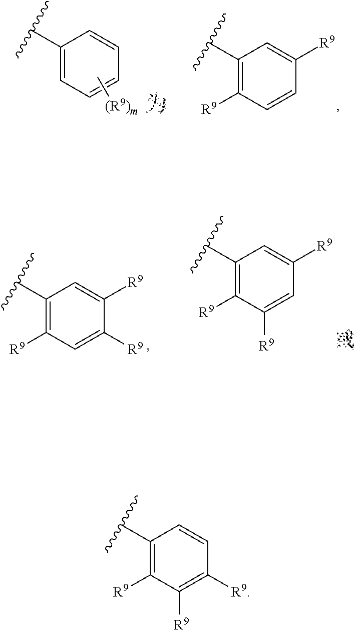

6. A triazine derivative or a pharmaceutically acceptable salt thereof as de scribed in formula (I′) of claim 1, characterized by the formula
(1) The triazine derivatives shown are any of the following compounds:

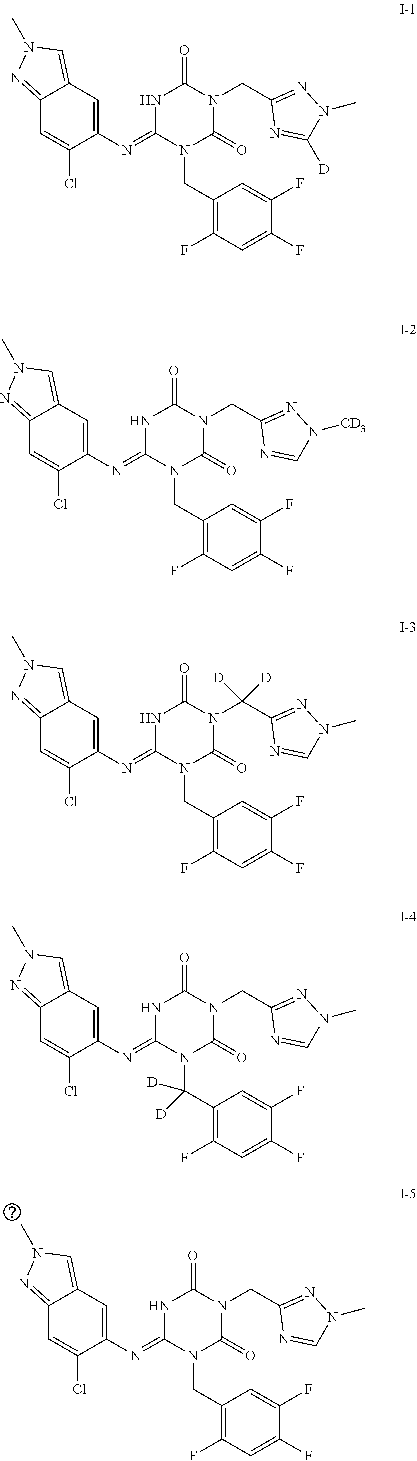

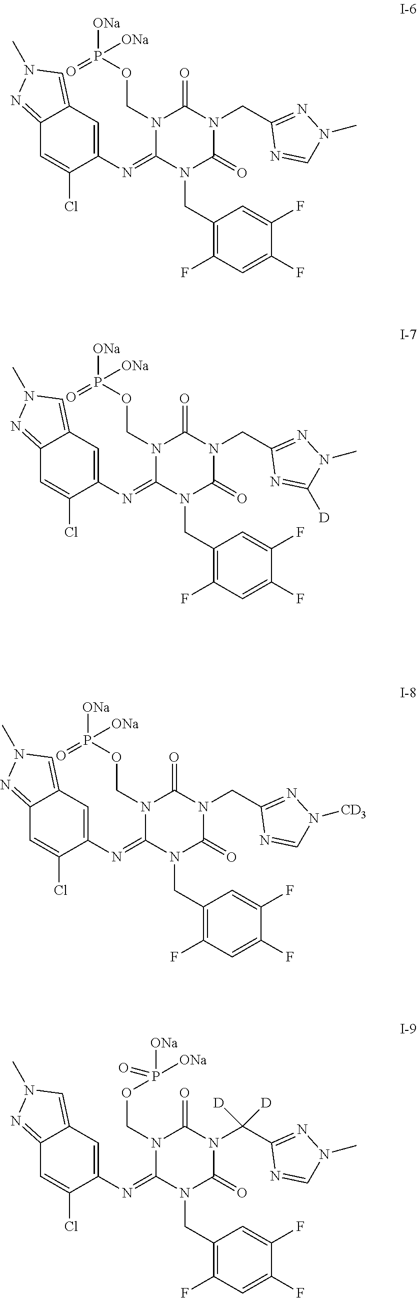

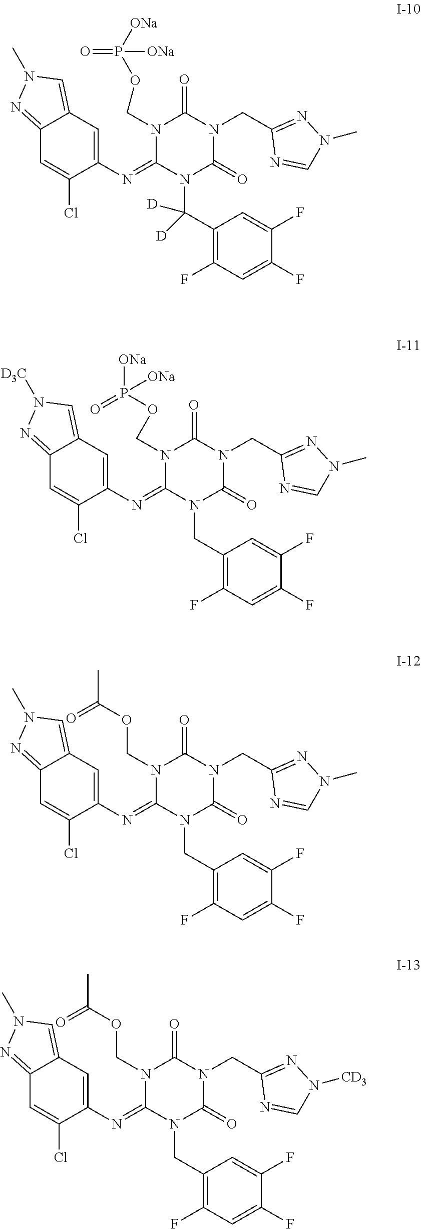

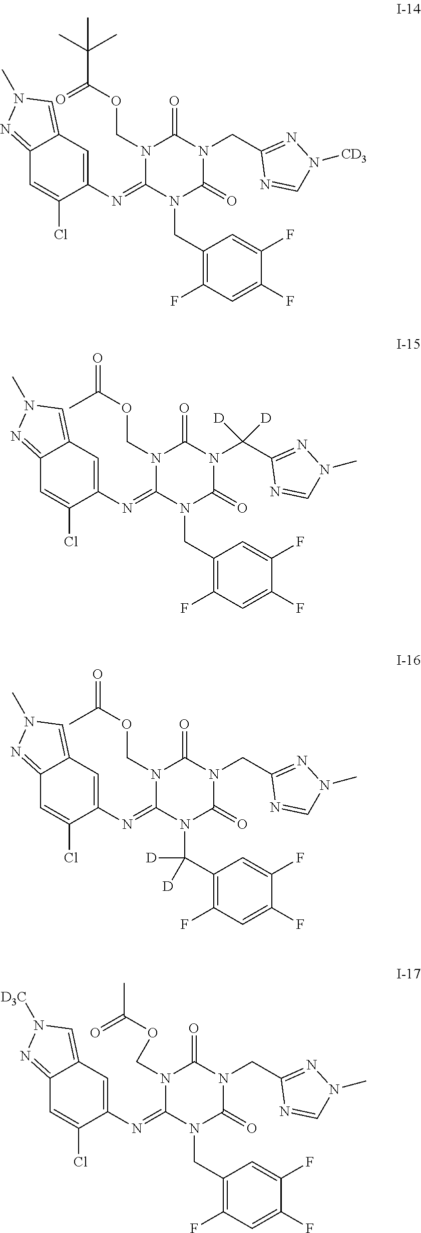

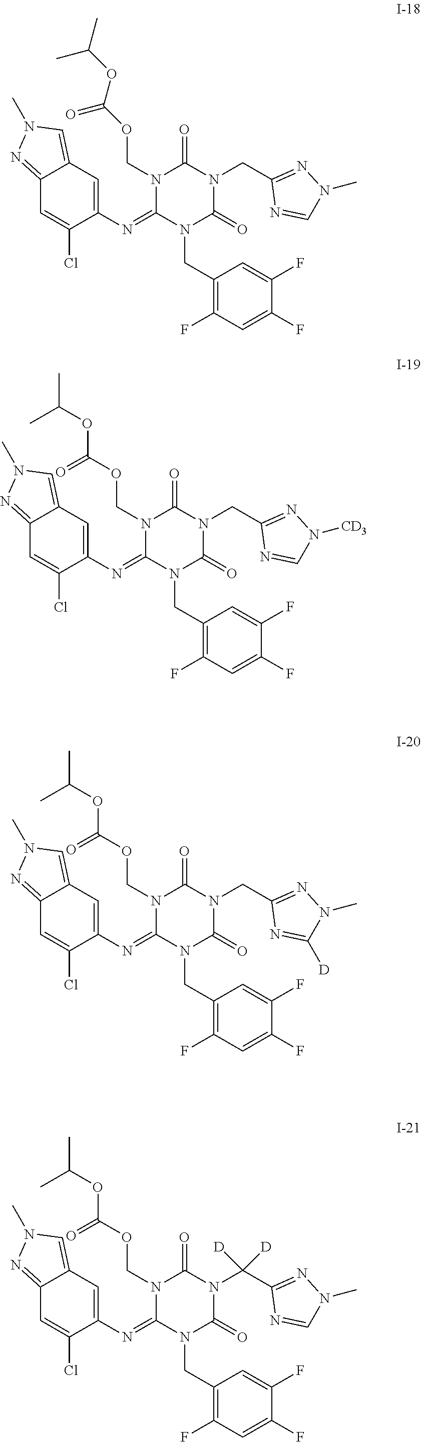

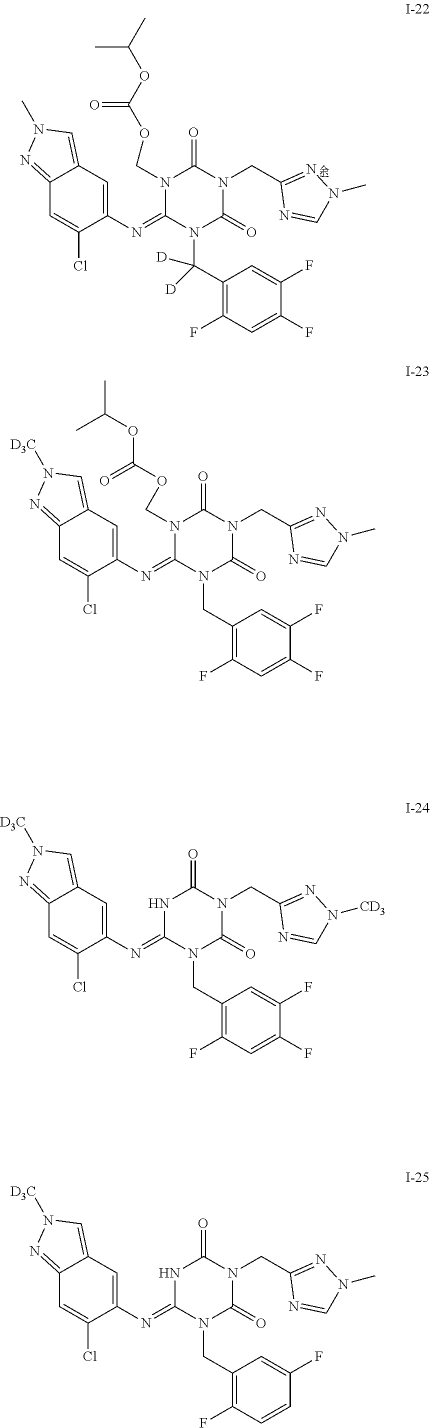

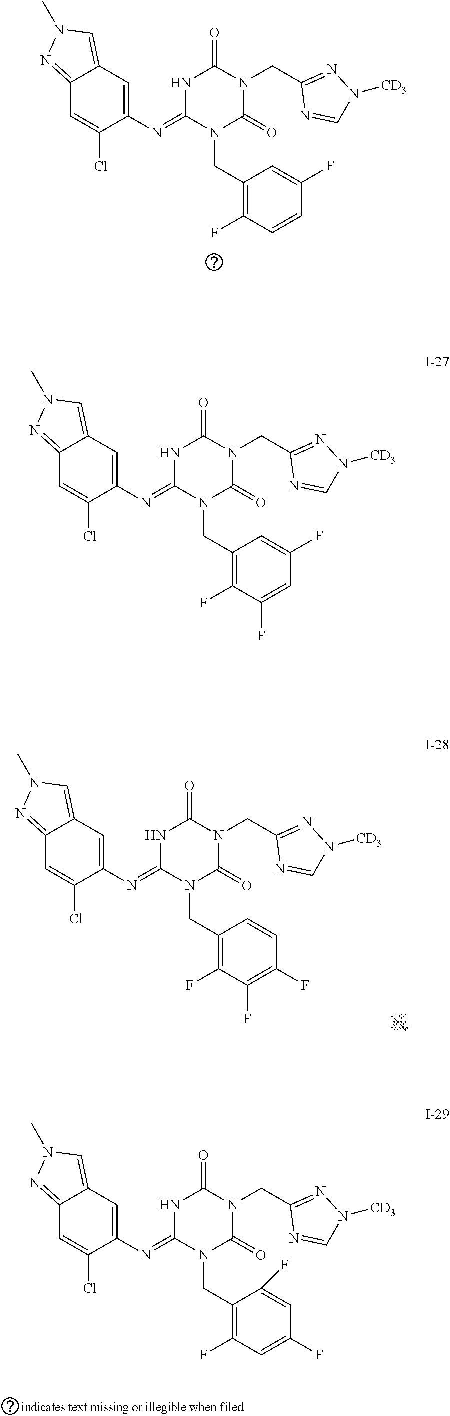 [SMILES: [2H]C([2H])([2H])n1cnc(Cn2c(=O)[nH]/c(=N\c3cc4cn(C)nc4cc3Cl)n(Cc3cc(F)ccc3F)c2=O)n1 ; [2H]C([2H])([2H])n1cnc(Cn2c(=O)[nH]/c(=N\c3cc4cn(C)nc4cc3Cl)n(Cc3cc(F)cc(F)c3F)c2=O)n1 ; [2H]C([2H])([2H])n1cnc(Cn2c(=O)[nH]/c(=N\c3cc4cn(C)nc4cc3Cl)n(Cc3ccc(F)c(F)c3F)c2=O)n1 ; [2H]C([2H])([2H])n1cnc(Cn2c(=O)[nH]/c(=N\c3cc4cn(C)nc4cc3Cl)n(Cc3c(F)cc(F)cc3F)c2=O)n1]

7. A triazine derivative, or a pharmaceutically acceptable salt thereof, as shown in formula (I′) of claim 1, is characterized in that the triazine derivative shown in formula (I′) is the triazine derivative shown in formula (I) or (III). 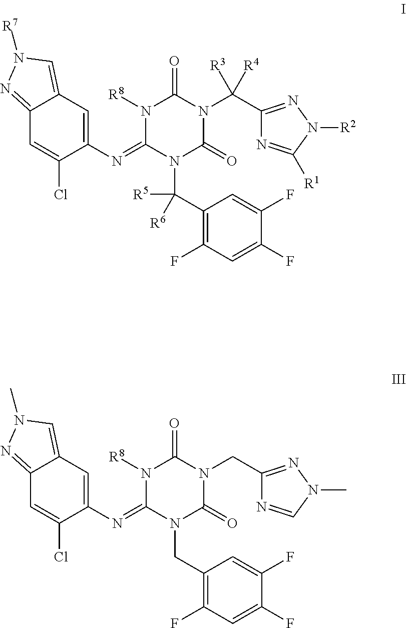
Where R¹ is hydrogen or deuterium.
R² is a methyl group or a methyl group replaced by 1, 2 or 3 deuterium.
R³ for hydrogen or deuterium.
R⁴ is hydrogen or deuterium.
R⁵ for hydrogen or deuterium.
R⁶ is hydrogen or deuterium.
R⁷ is a methyl group or a methyl group replaced by 1, 2 or 3 deuterium.
R⁸ is hydrogen, 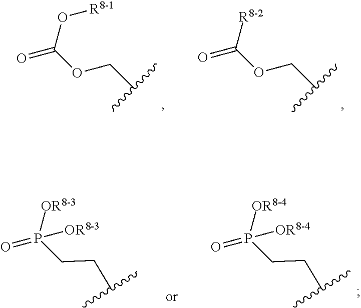
R⁸⁻¹ is C₁˜C₁₀ alkyl.
R⁸⁻² is C₁-C₁₀ alkyl.
Each R⁸⁻³ is independently hydrogen, C₁-C₁₀ alkyl, or sodium.
Each R⁸⁻⁴ is independently hydrogen, C₁-C₁₀ alkyl, or sodium.
Moreover, R¹, R², R³, R⁴, R⁵, R⁶ and R⁷ contain at least one deuterium.
Formula (III) is not

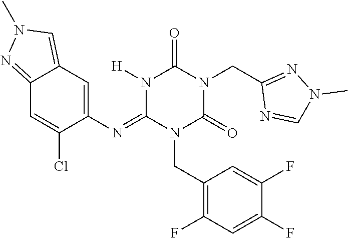

8. A triazine derivative or a pharmaceutically acceptable salt thereof as indicated in formula (I′) of claim 7 is characterized by a triazine derivative as indicated in formula (D) or (III) that satisfies one or more of the following conditions:
(1) In R⁸⁻¹, the mentioned C₁˜C₁₀ alkyl group is C₁˜C₆ alkyl groups, preferably methyl, ethyl, n-propyl, isopropyl, n-butyl, isobutyl, sec-butyl or tert-butyl groups, such as methyl, ethyl, isopropyl.
(2) In R⁸⁻², the mentioned C₁˜C₁₀ alkyl group is C₁˜C₆ alkyl groups, preferably methyl, ethyl, n-propyl, isopropyl, n-butyl, isobutyl, sec-butyl or tert-butyl, such as methyl, ethyl, isopropyl, n-butyl or tert-butyl.
(3) In R⁸⁻³, the mentioned C₁˜C₁₀ alkyl group is C₁˜C₆ alkyl groups, preferably methyl, ethyl, n-propyl, isopropyl, n-butyl, isobutyl, sec-butyl or tert-butyl.
(4) In R⁸⁻⁴, the mentioned C₁˜C₁₀ alkyl group is C₁˜C₆ alkyl groups, preferably methyl, ethyl, n-propyl, isopropyl, n-butyl, isobutyl, sec-butyl or tert-butyl.
(5) When R⁸ is Hydrogen, the three classes of derivatives shown in the formula (I′) may also be tautomers of them, e.g.

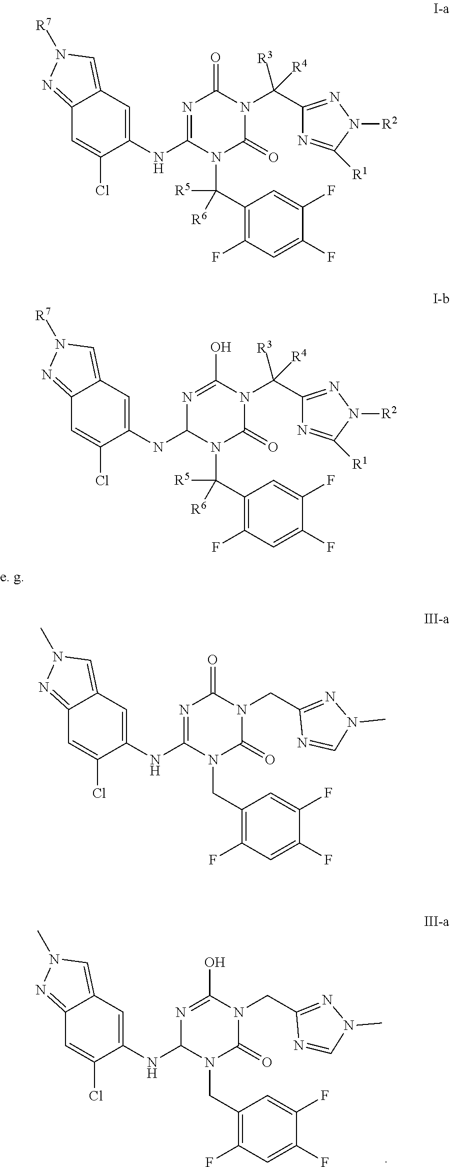

9. A triazine derivative, or a pharmaceutically acceptable salt thereof, as described in formula (I′) of claim 7, is characterized by the fact that a triazine derivative as described in formula (I) satisfies one or more of the following conditions:
(1) R² is methyl or —CD₃.
(2) R⁷ is methyl or —CD₃.
(3) R⁸⁻¹ is C1˜C6 alkyl.
(4) R⁸⁻² is C1˜C3 alkyl.
(5) R⁸⁻³ is sodium.
(6) R⁸⁻⁴ is sodium.
(7) Both R³ and R⁴ are deuterium.
And (8) R⁵ and R⁶ are deuterium simultaneously.

10. Triazine derivatives or pharmaceutically acceptable salts thereof, as indicated in formula (I′) of claim 7, are characterized by,
R⁸ is hydrogen,

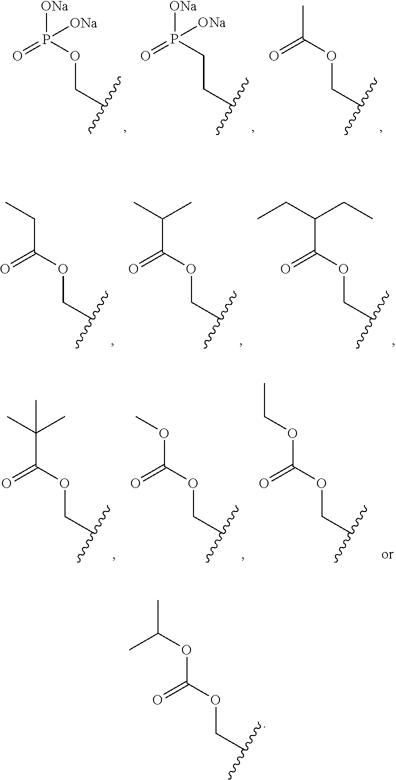

11. A triazine derivative or a pharmaceutically acceptable salt thereof as indicated in formula (I) of claim 7 is characterized by the fact that the triazine derivative as indicated in formula (I) is option 1, option 2, option 3, option 4 or option 5,
Option 1.
R¹ is tritium, R² is methyl, R³ is hydrogen, R⁴ is hydrogen, R⁵ is hydrogen, R⁶ is hydrogen, R⁷ is
methyl, R⁸ is hydrogen, 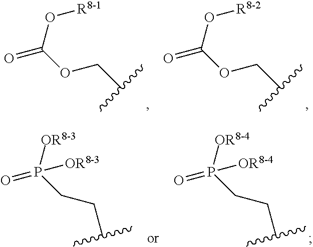
R⁸⁻¹ is C₁-C₁₀ alkyl, R⁸⁻² is C₁-C₁₀ alkyl, Each R⁸⁻³ is independently hydrogen, C₁-C₁₀ alkyl, or sodium, Each R⁸⁻⁴ is independently hydrogen, C₁-C₁₀ alkyl, or sodium.
Option 2.
R¹ is hydrogen, R² is —CD₃, R³ is hydrogen, R⁴ is hydrogen, R⁵ is hydrogen, R⁶ is hydrogen, R⁷ is
methyl, R⁸ is hydrogen, 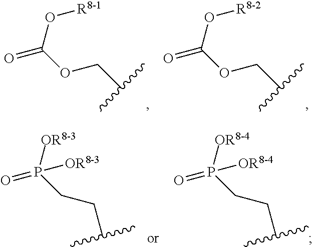
R⁸⁻¹ is C₁-C₁₀ alkyl, R⁸⁻² is C₁-C₁₀ alkyl, Each R⁸⁻³ is independently hydrogen, C₁-C₁₀ alkyl, or sodium,
Each R⁸⁻⁴ is independently hydrogen, C₁-C₁₀ alkyl, or sodium.
Option 3.
R¹ is hydrogen, R² is methyl, R³ is tritium, R⁴ is tritium, R⁵ is hydrogen, R⁶ is hydrogen,
R⁷ is methyl, R⁸ is hydrogen, 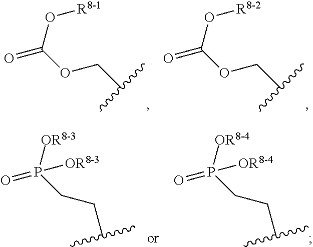
R⁸⁻¹ is C₁-C₁₀ alkyl. R⁸⁻² is C₁-C₁₀ alkyl. Each R⁸⁻³ is independently hydrogen, C₁-C₁₀ alkyl, or sodium. Each R⁸⁻⁴ is independently hydrogen, C₁-C₁₀ alkyl, or sodium,
Option 4.
R¹ is hydrogen, R² is methyl, R³ is hydrogen, R⁴ is hydrogen, R⁵ is tritium, R⁶ is tritium,
R⁷ is methyl, R⁸ is hydrogen, 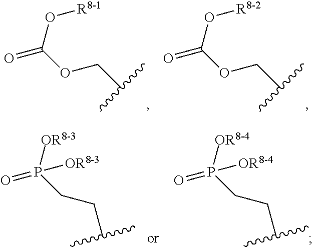
R⁸⁻¹ is C₁-C₁₀ alkyl, R⁸⁻² is C₁-C₁₀ alkyl. Each R⁸⁻³ is independently hydrogen, C₁-C₁₀ alkyl, or sodium. Each R⁸⁻⁴ is independently hydrogen, C₁-C₁₀ alkyl, or sodium.
Option 5
R¹ is hydrogen, R² is methyl, R³ is hydrogen, R⁴ is hydrogen, R⁵ is hydrogen, R⁶ is hydrogen,
R⁷ is —CD₃, R⁸ is hydrogen, 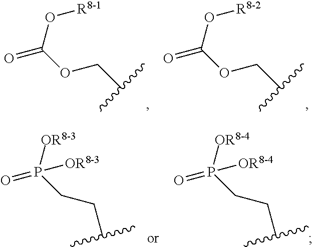
R⁸⁻¹ is C₁-C₁₀ alkyl, R⁸⁻² is C₁-C₁₀ alkyl. Each R⁸⁻³ is independently hydrogen, C₁-C₁₀ alkyl, or sodium. Each R⁸⁻⁴ is independently hydrogen, C₁-C₁₀ alkyl, or sodium.

12. A triazine derivative, or a pharmaceutically acceptable salt thereof, as shown in formula (I′) of claim 1, is characterized in that the triazine derivative shown in formula (I′) is the triazine derivative shown in VI 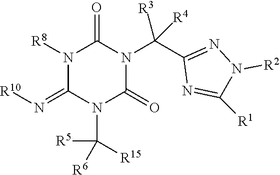
Where R¹ is hydrogen or deuterium,
R² is a methyl group or a methyl group replaced by 1, 2, or 3 tritium,
R³ is hydrogen or deuterium,
R⁴ is hydrogen or deuterium,
R⁵ is hydrogen or deuterium,
R⁶ is hydrogen or deuterium,
R⁸ is hydrogen, 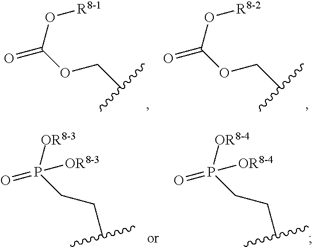
R⁸⁻¹ is C₁-C₁₀ alkyl,
R⁸⁻² is C₁-C₁₀ alkyl,
Each R⁸⁻³ is independently hydrogen, C₁-C₁₀ alkyl, or sodium,
Each R⁸⁻⁴ is independently hydrogen, C₁-C₁₀ alkyl, or sodium,
R¹⁰ is a 6-10 meta heteroaryl group that is not replaced or is replaced by one or more R¹⁰⁻¹; The type of heteroatom of the 6-10 meta heteroaryl group is selected from one or more of N, O and S, and the number of heteroatom is 1, 2 or 3;
Each R¹⁰⁻¹ is independently a C₁-C₆ alkyl, halogen, or deuterium that is not replaced or is replaced by one or more R¹⁰⁻¹⁻¹;
Each R¹⁰⁻¹⁻¹ is independently a halogen or deuterium,
R¹⁵ is

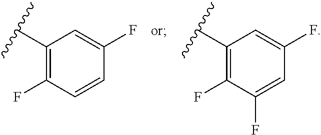 [SMILES: *c1cc(F)ccc1F ; *c1cc(F)cc(F)c1F]

13. A triazine derivative or a pharmaceutically acceptable salt thereof as indicated in formula (T′) of claim 12 is characterized by a triazine derivative as indicated in formula VI that satisfies one or more of the following conditions:
(1) In R⁸⁻¹, the mentioned C₁˜C₁₀ alkyl group is C₁˜C₆ alkyl groups, preferably methyl, ethyl, n-propyl, isopropyl, n-butyl, isobutyl, sec-butyl or tert-butyl groups, such as methyl, ethyl, isopropyl.
(2) In R⁸⁻², the mentioned C₁˜C₁₀ alkyl group is C₁˜C₆ alkyl groups, preferably methyl, ethyl, n-propyl, isopropyl, n-butyl, isobutyl, sec-butyl or tert-butyl, such as methyl, ethyl, isopropyl, n-butyl or tert-butyl.
(3) In R⁸⁻³, the mentioned C₁˜C₁₀ alkyl group is C₁˜C₆ alkyl groups, preferably methyl, ethyl, n-propyl, isopropyl, n-butyl, isobutyl, sec-butyl or tert-butyl.
(4) In R⁸⁻⁴, the mentioned C₁˜C₁₀ alkyl group is C₁˜C₆ alkyl groups, preferably methyl, ethyl, n-propyl, isopropyl, n-butyl, isobutyl, sec-butyl or tert-butyl.
(5) In R¹⁰, the 6-10 meta heteroaryl group is 5 and 6 meta heteroaryl group, and/or the 6-10 meta heteroaryl group is a heteroatom class of N and/or O, and/or the 6-10 meta heteroaryl group is a heteroatom number of 2; The 6-10 meta heteraryl group is preferably 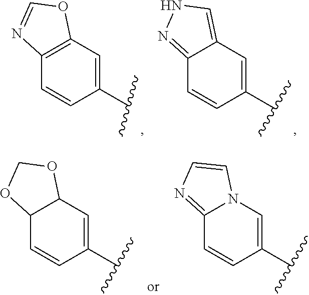 [SMILES: *c1ccc2ncoc2c1 ; *c1ccc2n[nH]cc2c1 ; *C1=CC2OCOC2C=C1 ; *c1ccc2nccn2c1]
(6) R¹⁰⁻¹, the mentioned C₁-C₆ alkyl group is methyl, ethyl, n-propyl, isopropyl, n-butyl, isobutyl, sec-butyl, or tert-butyl, such as methyl.
(7) the Halogen is fluorine, chlorine, bromine or iodine, such as fluorine or chlorine.
(8) R¹⁰⁻¹⁻¹, the halogen is atmosphere, chlorine, bromine or iodine, such as fluorine or chlorine.

14. A triazine derivative or a pharmaceutically acceptable salt thereof as indicated in formula (I′) of claim 12 is characterized by a triazine derivative as indicated in formula VI that satisfies one or more of the following conditions:
(1) R¹ is hydrogen.
(2) R² is methyl or —CD₃.
(3) R³ is hydrogen.
(4) R⁴ is hydrogen.
(5) R⁵ is hydrogen.
(6) R⁶ is hydrogen.
(7) each R¹⁰⁻¹ is independently chlorine, methyl, fluorine, or —CD₃.
And in (8) R¹⁰, the 6-10 meta heteroaryl group is, 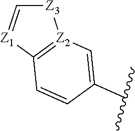
Z₁ is N or O; Z₂ is N or C; Z₃ is C or O. In a better way, R¹⁰ is 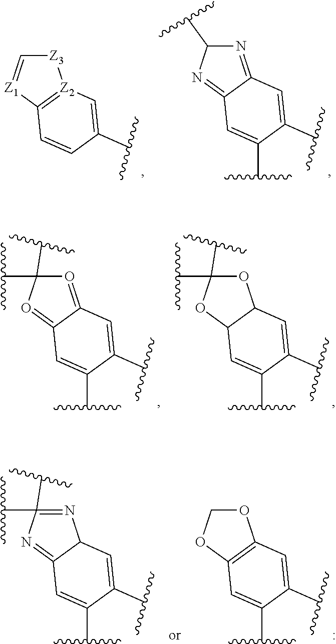
Another example

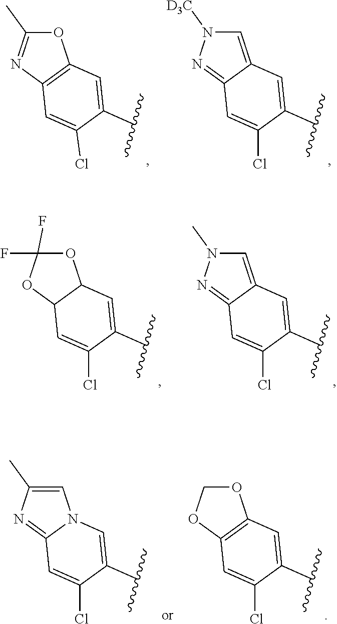 [SMILES: *c1cc2oc(C)nc2cc1Cl ; *c1cc2cn(C([2H])([2H])[2H])nc2cc1Cl ; *C1=CC2OC(F)(F)OC2C=C1Cl ; *c1cc2cn(C)nc2cc1Cl ; *c1cn2cc(C)nc2cc1Cl ; *c1cc2c(cc1Cl)OCO2]

15. A binary coproduct of a triazine derivative with an acid as indicated in formula I′ of claim 1. the acid is malic acid, maleic acid, citric acid, ascorbic acid, mandelic acid, tartaric acid, fumaric acid or acetic acid, preferably fumaric acid or acetic acid.
Preferably, the binary eutectic is or 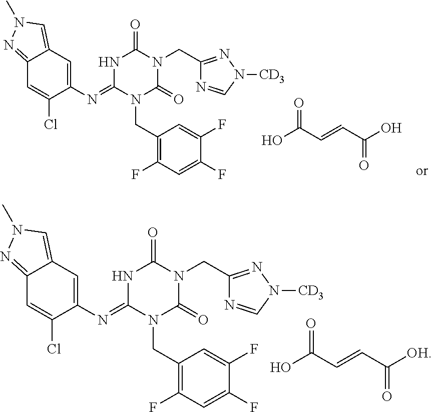
Preferably, the binary coproduct 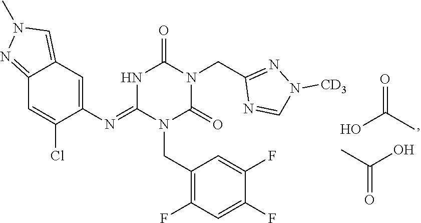
which is represented by an X-ray powder at 2θ angles, diffraction peaks are found at 5.4±0.2, 19.8±0.2, 22.2±0.2, 25.5±0.2. The X-ray powder diffraction pattern represented by 2θ angles also has diffraction peaks at one or more of 9.9±0.2, 10.7±0.2, 12.6±0.2, 27.3±0.2 and 28.0±0.2. Preferably, its X-ray powder diffraction pattern expressed in 2θ angles is shown in FIG. 1:
Preferably, the binary eutectic 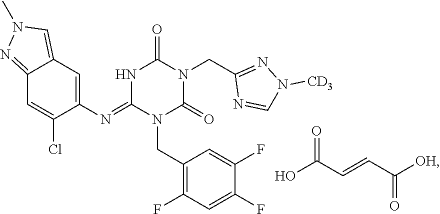
which is represented by 2θ angles in X-rays, the powder diffraction pattern has diffraction peak at 7.85±0.2°, 9.55±0.2°, 12.01±0.2°, 13.88±0.2°, 14.79±0.2°, 17.19±0.2°, 18.71±0.2°, 19.16±0.2°, 23.60±0.2°, 23.85±0.2°, 24.76±0.2°, 28.95±0.2°. Preferably, the X-ray powder diffraction pattern represented by 2θ angles also has diffraction peaks at one or more places of 6.05±0.2°, 11.02±0.2°, 11.60±0.2°, 12.39±0.2°, 13.43±0.2°, 15.21±0.2°, 16.41±0.2°, 18.13±0.2°, 19.59±0.2°, 19.91±0.2°, 20.43±0.2°, 20.97±0.2°, 21.53±0.2°, 22.04±0.2°, 22.70±0.2°, 23.16±0.2°, 25.47±0.2°, 27.13±0.2°, 27.77±0.2°, 28.28±0.2°, 29.76±0.2°, 31.17±0.2°, 32.11±0.2°, 32.64±0.2°, 33.34±0.2°, 34.03±0.2°, and 35.00±0.2°; Preferably, its X-ray powder diffraction pattern expressed at 2θ angles is shown in FIG. 2.

16. A ternary coproduct formed by a triazine derivative with niacinamide and acid as indicated in formula I′ of claim 1. The acids are malic acid, maleic acid, citric acid, ascorbic acid, mandelic acid, tartaric acid, fumaric acid or acetic acid, fumaric acid is preferred.
Preferably, the ternary eutectic is 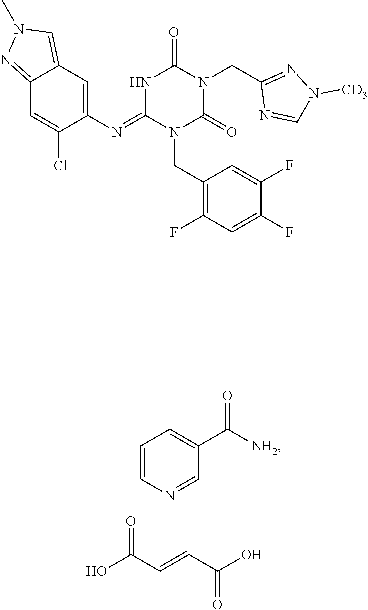
Preferably, the ternary eutectic is 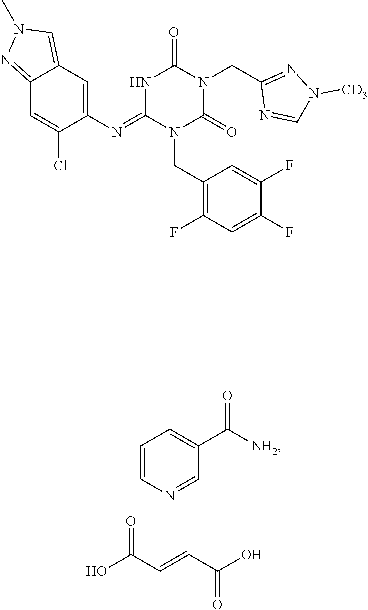
its X-ray powder diffraction pattern represented by 2θ angles has diffraction peaks at 11.2±0.2°, 15.7±0.2°, 18.9±0.2°, 19.3±0.2°, 22.9±0.2°, 24.0±0.2°, 24.9±0.2° and 29.6±0.2°; Preferably, It also has diffraction peaks at one or more places of 10.4±0.2°, 11.8±0.2°, 12.2±0.2°, 12.6±0.2°, 13.6±0.2°, 14.1±0.2°, 15.0±0.2°, 17.4±0.2°, 18.2±0.2°, 18.5±0.2°, 19.8±0.2°, 20.1±0.2°, 20.3±0.2°, 20.6±0.2°, 21.2±0.2°, 21.7±0.2°, 22.2±0.2°, 22.5±0.2°, 23.3±0.2°, 23.8±0.2°, 25.6±0.2°, 26.0±0.2°, 27.6±0.2°, 28.0±0.2°, 28.9±0.2°, 29.7±0.2°, 29.9±0.2°, 31.4±0.2°, 32.8±0.2°, 33.5±0.2°, 35.1±0.2°, 36.3±0.2° and 39.6±0.2°; Preferably, its X-ray powder diffraction pattern expressed at 2θ angles is shown in FIG. 3.

17. A pharmaceutical composition characterized in that it includes substance B′ and one or more pharmaceutically acceptable carriers; Substance B′ is a triazine derivative or a pharmaceutically acceptable salt thereof as described in formula I of claim 1; The amount of substance B′ may be a therapeutic effective amount.

18. The use of a substance B′ in the preparation of an antiviral drug, which is a triazine derivative or a pharmaceutically acceptable salt thereof as described in formula I of claim 1. The viruses are coronaviruses, influenza viruses, respiratory syncytial viruses, flaviviridae viruses, filoviridae viruses or porcine Epidemic diarrhea virus (PEDV). Preferably, the coronavirus is selected from one or more of MERS-CoV, SARS-CoV, and SARS-CoV-2, and the coronavirus is preferred as SARS-CoV-2.

19. The application of a substance B′ in the preparation of drugs for the treatment and/or prevention of coronavirus-related diseases, which is a triazine derivative or its pharmaceutically acceptable salt as indicated in Formula I′ of claim 1.

20. A method of preparing triazine derivatives shown in formula (I′) is characterized by method 1, method 2, method 3, method 4, or method 5.
The method 1 comprises the following steps where, in a solvent (e.g., anhydrous tetrahydrofuran), in the presence of a base (e.g., Hexamethyldisilylamine lithium), a compound shown in formula I′-S1 reacts with heavy water as shown below to obtain a compound shown in formula I′. 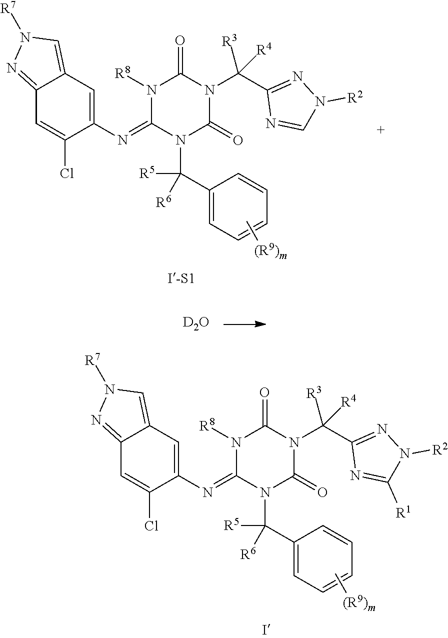
Where R¹ is deuterium; R², R³, R⁴, R⁵, R⁶, R⁷ and R⁸ are defined in claim 1.
Method 2 consists of the following steps: In a solvent (e.g., a mixed solution of acetic acid and tert-butanol), a compound shown in formula I′-S2 reacts with a compound shown in formula I′-S3 as shown below to obtain a compound shown in formula I′. 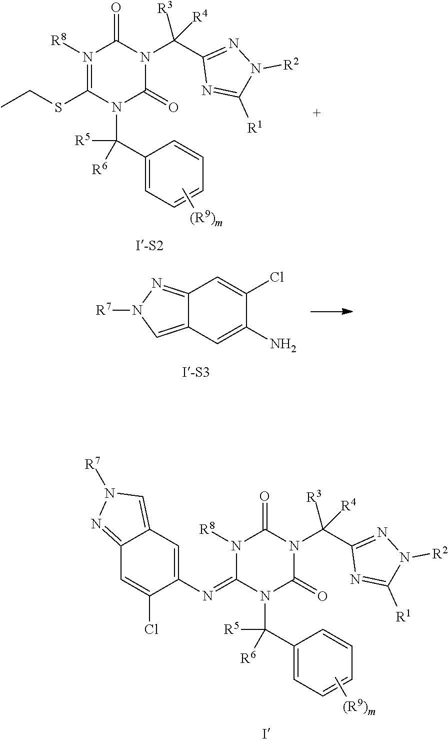
Where R¹, R², R³, R⁴, R⁵, R⁶, R⁷, R⁸, R⁸⁻¹, R⁸⁻², each R⁸⁻³ and each R⁸⁻⁴ are defined in claim 1.
Method 3 consists of the following steps in which, in a solvent (e.g., N, N-dimethylformamide or dimethylacetamide) and in the presence of a base (e.g., anhydrous cesium carbonate or potassium carbonate), a compound shown in formula I′-S4 reacts with a compound shown in formula I′-S5 as shown below to obtain a compound shown in formula I′. 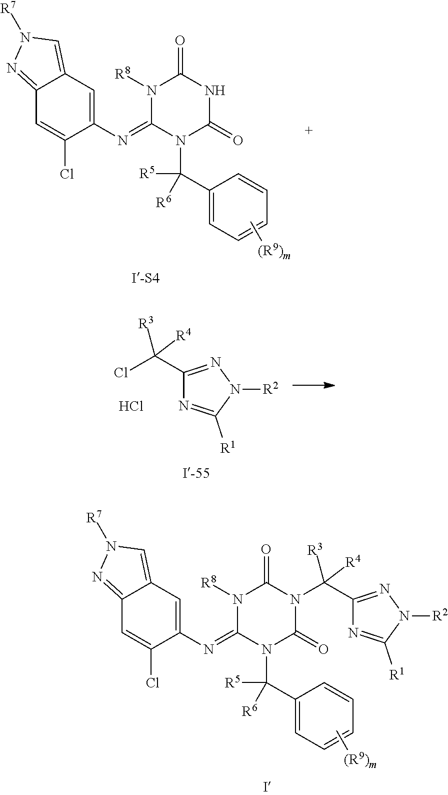
Where R¹, R², R³, R⁴, R⁵, R⁶, R⁷, R⁸, R⁸⁻¹, R⁸⁻², each R⁸⁻³ and each R⁸⁻⁴ are defined in claim 1.
The method 4 consists of the following steps, in a solvent (e.g., dichloromethane), a compound shown in formula I′-S6 reacts with sodium hydroxide (e.g., an ethanol solution of sodium hydroxide) as shown below to obtain a compound shown in formula I′ 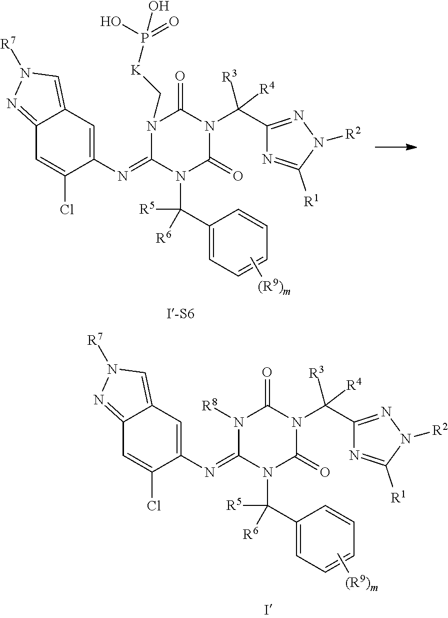
Where K is or CH₂, R⁸ is 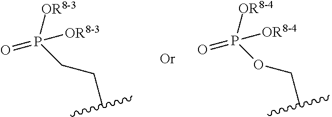
Each R⁸⁻³ and each R⁸⁻⁴ are sodium, Where R¹, R², R³, R⁴, R⁵, R⁶ and R⁷ are defined in claim 1.
Method 5 consists of the following steps. In a solvent (e.g., N, N-dimethylformamide or dimethylacetamide), in the presence of a base (e.g., sodium hydride), a compound shown in formula I′-S7 reacts with a compound shown in formula I′-S8 as shown below to obtain a compound shown in formula I′, 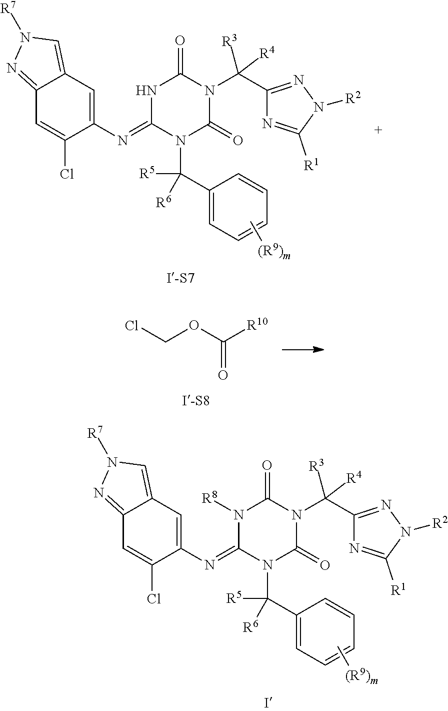
Where R¹⁰ is C₁˜C₁₀ alkyl or —OC₁˜C₁₀ alkyl, R⁸ is 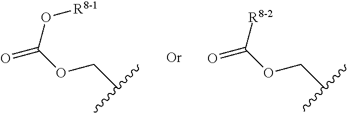
Each R⁸⁻¹ and each R⁸⁻² are independently C₁˜C₁₀ alkyl. R¹, R², R³, R⁴, R⁵, R⁶, R⁷ are defined in claim 1.
